(19) United States
(12) Patent Application Publication
(10) Pub. No.: US 2022/0291203 A1
(43) Pub. Date: Sep. 15, 2022

(54) METHODS OF MEASURING CELL-MEDIATED KILLING BY EFFECTORS

(71) Applicant: IMMUNOWAKE INC., Birmingham, AL (US)

(72) Inventors: Ellen WU, Birmingham, AL (US); Xiaoyun WU, Birmingham, AL (US); John WAKEFIELD, Birmingham, AL (US)

(21) Appl. No.: 17/629,730

(22) PCT Filed: Jul. 24, 2020
(86) PCT No.: PCT/US2020/043615

§ 371 Date: Jan. 24, 2022

Related U.S. Application Data
(60) Provisional application No. 62/878,717, filed on Jul. 25, 2019.

(51) Int. Cl. G01N 33/50

(52) U.S. Cl. CPC G01N 33/5011; G01N 33/5014; G01N 33/5082; G01N 2800/7028

(57) ABSTRACT

The disclosure provides for compositions, methods, and kits for evaluating the effect of a cell-killing agent on a population of tumor cells (e.g., tumor cells that can inducibly express reporter protein).

### CROSS REFERENCE TO RELATED APPLICATIONS

This application claims priority benefit from U.S. Provisional Application No. 62/878,717 filed on Jul. 25, 2019, the content of which is incorporated herein by reference in its entirety.

### FIELD OF THE INVENTION

This disclosure provides for compositions, methods, and kits for evaluating the effect of a cell-killing agent on a population of tumor cells (e.g., tumor cells that can inducibly express reporter protein).

### BACKGROUND OF THE INVENTION

Cell culture can be used as a model for studying disease processes, such as cancer, and for testing potential therapeutic agents used in the treatment thereof. Cells cultured in monolayers may not adequately mimic the in vivo environment from which the cells were originally isolated. This is because disease pathogenesis can be influenced by the context of three-dimensional (3D) tissue structures, which can involve interactions between different cell types in the stromal and epithelial compartments and with the extracellular matrix (Hanahan and Weinberg, Cell. 144(5) 646-74. 2011). Co-cultured cells grown in three-dimensional (e.g., spheroid) structures represent an in vivo biological environment much more faithfully than cells grown in a two-dimensional (2D) monolayer, and include factors such as cell morphology, growth kinetics, gene expression, and response to drugs (Burdett et al, Tissue Engineering: Part B Vol 16, No. 3 (2010), 351-9.; Mehta et al, J. Control. Release 164(2) 192-204 (2012)). Therefore, establishing a cell killing assay under spheroid conditions may provide a useful tool to screen for and evaluate new therapeutic compounds and immunotherapy candidates.

Some methods for measuring cell-killing involve fluorescent imaging. Cell viability is inversely correlated with the fluorescent signal. Accurately quantifying cell killing under three-dimensional conditions using these reporters are difficult because spheroids are not easily imaged. Furthermore, the addition of different cell populations, such as stromal cells, into the spheroid dilute the fluorescent signal and decrease the sensitivity of the detection. New methods are needed to sensitively and reproducibly detect cell killing by cell killing agents in three-dimensional culture systems.

Secreted reporter systems offer an alternative to current reporter methods. Secreted reporter proteins accumulate in the cell culture medium and can be used to monitor the assay over multiple time points. A previous study established secrted luciferase as a sensitive and real-time reporter for cell viability, showing that a linear relationship between cell viability and luciferase luminescence was consistent in values for as few as 40 cells (Lupold et al., (2012) A Real Time Metridia Luciferase Based Non-Invasive Reporter Assay of Mammalian Cell Viability and Cytotoxicity via the β-actin Promoter and Enhancer. PLoS ONE 7(5)). However, the continuous accumulation of secreted reporter proteins in the media over time could dampen the sensitivity and/or affect the accuracy of cytotoxicity assays.

### BRIEF SUMMARY OF THE INVENTION

The present application provides novel assay methods and systems for evaluating the effectiveness of cell-killing agents.

In one aspect, the disclosure provides for a method of evaluating the effectiveness of a cell-killing agent (e.g., small compound, immune effector cell, antibody such as multispecific antibody. ADC, immunomodulator such as immune checkpoint inhibitor, etc., or any combinations thereof) on a population of tumor cells, the method comprising: a) contacting the tumor cells with a cell-killing agent, wherein each of the tumor cells comprises a nucleic acid encoding a reporter protein (e.g., luciferase or GFP); b) allowing expression of the nucleic acid to produce the reporter protein; and c) determining the amount of the reporter protein, wherein the amount of the reporter protein negatively correlates with the effectiveness of the cell killing agent.

In one aspect, the disclosure provides for a method of evaluating the effectiveness of a cell-killing agent (e.g., small compound, immune effector cell, antibody such as multispecific antibody, ADC, immunomodulator such as immune checkpoint inhibitor, etc., or any combinations thereof) on a population of tumor cells, the method comprising: a) contacting the tumor cells with a cell-killing agent, wherein each of the tumor cells comprises a nucleic acid encoding a reporter protein (e.g., luciferase or GFP), wherein the expression of the nucleic acid is controlled by an inducible promoter (e.g., TetOn); b) inducing expression of the nucleic acid to produce the reporter protein; and c) determining the amount of the reporter protein, wherein the amount of the reporter protein negatively correlates with the effectiveness of the cell killing agent.

In some embodiments according to any of the methods described above, the contacting step is carried out at a cell-killing phase, and the determining step is carried out at a subsequent evaluating phase.

In some embodiments according to any of the methods described above, the contacting step occurs after (e.g., about 2 to about 48 hours after, or about 12 to about 24 hours after) the inducing step.

In some embodiments according to any of the methods described above, the contacting step occurs simultaneously with the inducing step.

In some embodiments according to any of the methods described above, the contacting step occurs before (e.g., about 2 to about 48 hours before, about 4 to about 48 hours before, about 4 to about 24 hours before, or about 24 to about 48 hours before) the inducing step. In some embodiments, the contacting step occurs for at least about 24 hours prior to the inducing step. In some embodiments, the contacting step occurs for about 4 to about 48 hours (such as about 4 to about 8 hours, about 24 to about 48 hours, about 4 to about 24 hours, or about 12 to about 24 hours) prior to the inducing step. In some embodiments, the contacting step occurs for up to about 6 days (e.g., about any of 1, 2, 3, 4, 5, or 6 days) prior to the inducing step. In some embodiments, the inducing step occurs for about 4 to about 48 hours (e.g., about 4 to about 8 hours, about 12 to about 48 hours, about 24 to about 48 hours, or about 12 to about 24 hours).

In some embodiments according to any of the methods described above, the inducing step comprises treating the tumor cells with an induction agent, such as an induction agent selected from the group consisting of: tetracycline, doxycycline, estrogen receptor, and cumate, or any combination thereof.

In some embodiments according to any of the methods described above, the reporter protein is secreted by the tumor cells. In some embodiments, the reporter protein is selected from the group consisting of luciferase, secreted alkaline phosphatase, and secreted fluorescent protein, or any combination thereof. In some embodiments, the reporter protein is luciferase, such as luciferase selected from the group consisting of Oplophorus luciferase, beetle luciferase, Renilla luciferase, Metridia luciferase, Gaussia luciferase, and NANOLUC luciferase, or any combination thereof.

In some embodiments according to any of the methods described above, the determining step comprises detecting the reporter protein over different time points.

In some embodiments according to any of the methods described above, the tumor cells are present in a mixture comprising a second population of cells, such as a second population of cells selected from the group consisting of fibroblast cells, stromal cells, endothelial cells, tumor associated macrophages, myeloid-derived suppressive cells, or any combination/variant thereof, or any combination thereof. In some embodiments, the second population of cells are fibroblast cells.

In some embodiments according to any of the methods described above, the tumor cells are present in a 3D spheroid or a 2D monolayer.

In some embodiments according to any of the methods described above, the cell-killing agent is selected from the group consisting of: a cytotoxin, a drug, a small molecule, and a small molecule compound, or any combination thereof.

In some embodiments according to any of the methods described above, the cell-killing agent is an immune cell, such as an immune cell selected from the group consisting of a natural killer (NK) cell, a natural killer T (NKT) cell, a T cell (e.g., CTL), CAR-T cell, a CD14+ cell, a dendritic cell, and a PBMC cell, or any combination thereof. In some embodiments, the cell-killing agent is an NK cell, a T cell (e.g., CTL), or a PBMC.

In some embodiments according to any of the methods described above, the cell-killing agent is an immunomodulating agent, and the contacting step is conducted in the presence of an immune cell. In some embodiments, the immune cell is selected from the group consisting of a natural killer (NK) cell, a natural killer T (NKT) cell, a T cell (e.g., CTL), CAR-T cell, a CD14+ cell, a dendritic cell, and a PBMC cell, or any combination thereof. In some embodiments, the immunomodulating agent is an immune checkpoint inhibitor (e.g., antibody). In some embodiments, the immune checkpoint inhibitor inhibits an inhibitory checkpoint molecule selected from the group consisting of PD-1, PD-L1, PD-L2, Siglec, BTLA, and CTLA-4, or any combination thereof. In some embodiments, the cell-killing agent is an immune checkpoint inhibitor (e.g., antibody) that inhibits PD-1 or PD-L1.

In some embodiments according to any of the methods described above, the cell-killing agent is an antibody, such as an antibody selected from the group consisting of an anti-PD-1 antibody (e.g., nivolumab such as Opdivo®, pembrolizumab, or cemiplimab), an anti-PD-L1 antibody (e.g., atezolizumab, avelumab, or durvalumab), an anti-CD47 antibody, an anti-HER2 antibody (e.g., trastuzmab such as Herceptin®), an anti-CD20 antibody, and an anti-CD3 antibody, or any combination thereof. In some embodiments, the antibody is monospecific (e.g., anti-PD-1 antibody such as nivolumab, anti-HER2 antibody such as trastuzmab, or anti-PD-L1 antibody such as atezolizumab or durvalumab). In some embodiments, the antibody is multispecific, such as an anti-HER2/anti-CD3 antibody, an anti-HER2/anti-CD47/anti-CD3 antibody, or an anti-PD-L1/anti-CD47/anti-CD3 antibody.

In some embodiments according to any of the methods described above, further comprising contacting the tumor cells with a second cell-killing agent. In some embodiments, the second cell-killing agent is an immune checkpoint inhibitor, such as an immune checkpoint inhibitor (e.g., antibody) that inhibits an inhibitory checkpoint molecule selected from the group consisting of PD-1, PD-L1, PD-L2, Siglec, BTLA, and CTLA-4, or any combination thereof. In some embodiments, the second cell-killing agent is an antibody, such as an anti-PD-1 antibody or an anti-PD-L1 antibody. In some embodiments, the second cell-killing agent is an siRNA, a CRISPR/Cas, a ZFN, or a TALEN construct (“KO construct”) targeting the inhibitory checkpoint molecule (e.g., PD-L1), e.g., transduced into the tumor cells. In some embodiments, the second cell-killing agent is an immune cell selected from the group consisting of a natural killer (NK) cell, a natural killer T (NKT) cell, a T cell (e.g., CTL), CAR-T cell, a CD14+ cell, a dendritic cell, and a PBMC cell, or any combination thereof. In some embodiments, the contacting of the second cell-killing agent occurs simultaneously with the contacting of the cell-killing agent. In some embodiments, the contacting of the second cell-killing agent occurs after (e.g., about 5 min to about 48 hours after, or about 2 hours to about 24 hours after) contacting of the cell-killing agent, but before the inducing step. In some embodiments, the contacting of the second cell-killing agent occurs before (e.g., about 5 min to about 48 hours before, or about 2 hours to about 24 hours before) contacting of the cell-killing agent. In some embodiments, the second cell-killing agent is the same as the cell-killing agent. In some embodiments, the second cell-killing agent (e.g., anti-HER2 antibody, anti-PD-1 antibody, anti-PD-L1 antibody, anti-HER2/anti-CD3 antibody, anti-HER2/anti-CD47/anti-CD3 antibody, or anti-PD-L1/anti-CD47/anti-CD3 antibody) is different from the cell-killing agent (e.g., NK cells, T cells such as CTLs, or PBMCs).

In some embodiments according to any of the methods described above, the nucleic acid encoding the reporter protein (e.g., luciferase or GFP) is introduced into the tumor cells by a retroviral or lentiviral vector system.

In some embodiments according to any of the methods described above, each of the tumor cells further comprise a second nucleic acid encoding a second reporter protein (e.g., luciferase or GFP), such as GFP. In some embodiments, the expression of the second nucleic acid is also controlled by the inducible promoter (e.g., TetOn), i.e., both the nucleic acid encoding the reporter protein and the second nucleic acid encoding the second reporter protein are under the same promoter control. In some embodiments, the expression of the second nucleic acid is controlled by a second inducible promoter (e.g., TetOn). In some embodiments, the inducible promoter and the second inducible promoter are the same (e.g., both are TetOn promoters). In some embodiments, the inducible promoter and the second inducible promoter are different. In some embodiments, the second nucleic acid encoding the second reporter protein and the nucleic acid encoding the reporter protein are on the same vector, either under same promoter control, or under controls of different promoters. In some embodiments, the second nucleic acid encoding the second reporter protein and the nucleic acid encoding the reporter protein are on different vectors. In some embodiments, the second reporter protein is the same as the report protein.

In one aspect, the disclosure provides for a composition comprising a population of tumor cells, wherein each of the tumor cells comprise a nucleic acid encoding a reporter protein (e.g., luciferase or GFP), wherein the expression of the nucleic acid is controlled by an inducible promoter (e.g., TetOn). In some embodiments, the reporter protein is secreted by the tumor cells.

In some embodiments according to any of the compositions described above, the reporting protein is selected from the group consisting of luciferase, secreted alkaline phosphatase, and secreted fluorescent protein, or any combination thereof. In some embodiments, the reporting protein is a luciferase selected from the group consisting of: Oplophorus luciferase, beetle luciferase, Renilla luciferase, Metridia luciferase, Gaussia luciferase, and NANOLUC luciferase, or any combination thereof.

In some embodiments according to any of the compositions described above, the composition further comprises a second population of cells, such as a second population of cells selected from the group consisting of fibroblast cells, stromal cells, endothelial cells, tumor associated macrophages, myeloid-derived suppressive cells, or any combination/variant thereof, or any combination thereof. In some embodiments, the second population of cells are fibroblasts.

In some embodiments according to any of the compositions described above, the composition is a 3D spheroid or a 2D monolayer.

In some embodiments according to any of the compositions described above, the composition further comprises a cell killing agent. In some embodiments, the cell-killing agent is selected from the group consisting of: a cytotoxin, a drug, a small molecule, and a small molecule compound, or any combination thereof. In some embodiments, the cell-killing agent is an immune cell. In some embodiments, the cell-killing agent is an immunomodulating agent, and the composition further comprises an immune cell. In some embodiments, the immune cell is selected from the group consisting of an NK cell, an NKT cell, a T cell, a CAR-T cell, a CD14+ cell, a dendritic cell, and a PBMC cell, or any combination thereof. In some embodiments, the immunomodulating agent is an immune checkpoint inhibitor (e.g., antibody), such as an immune checkpoint inhibitor that inhibits an inhibitory checkpoint molecule selected from the group consisting of PD-1, PD-L1, PD-L2, Siglec, BTLA, and CTLA4, or any combination thereof. In some embodiments, the cell-killing agent is an antibody. In some embodiments, the antibody is selected from the group consisting of an anti-PD-1 antibody (e.g., nivolumab such as Opdivo®, pembrolizumab, or cemiplimab), an anti-PD-L1 antibody (e.g., atezolizumab, avelumab, or durvalumab), an anti-CD47 antibody, an anti-HER2 antibody (e.g., trastuzmab such as Herceptin®), an anti-CD20 antibody, and an anti-CD3 antibody, or any combination thereof. In some embodiments, the antibody is monospecific (e.g., anti-PD-1 antibody such as nivolumab, anti-HER2 antibody such as trastuzmab, or anti-PD-L1 antibody such as atezolizumab or durvalumab). In some embodiments, the antibody is multispecific, such as an anti-HER2/anti-CD3 antibody, an anti-HER2/anti-CD47/anti-CD3 antibody, or an anti-PD-L1/anti-CD47/anti-CD3 antibody.

In some embodiments according to any of the compositions described above, the composition further comprises a second cell-killing agent. In some embodiments, the second cell-killing agent is an immune checkpoint inhibitor, such as an immune checkpoint inhibitor (e.g., antibody) that inhibits an inhibitory checkpoint molecule selected from the group consisting of PD-1, PD-L1, PD-L2, Siglec, BTLA, and CTLA-4, or any combination thereof. In some embodiments, the second cell-killing agent is an antibody, such as an anti-PD-1 antibody or an anti-PD-L1 antibody. In some embodiments, the second cell-killing agent is an immune cell selected from the group consisting of a natural killer (NK) cell, a natural killer T (NKT) cell, a T cell (e.g., CTL), CAR-T cell, a CD14+ cell, a dendritic cell, and a PBMC cell, or any combination thereof. In some embodiments, the second cell-killing agent is the same as the cell-killing agent. In some embodiments, the second cell-killing agent (e.g., anti-HER2 antibody, anti-PD-1 antibody, anti-PD-L1 antibody, anti-HER2/anti-CD3 antibody, anti-HER2/anti-CD47/anti-CD3 antibody, or anti-PD-L1/anti-CD47/anti-CD3 antibody) is different from the cell-killing agent (e.g., NK cells, T cells such as CTLs, or PBMCs).

In some embodiments according to any of the compositions described above, the composition further comprises an induction agent selected from the group consisting of: tetracycline, doxycycline, estrogen receptor, and cumate, or any combination thereof. In some embodiments, the induction agent is doxycycline.

In some embodiments according to any of the compositions described above, the composition further comprises the reporter protein (e.g., luciferase or GFP) secreted by the tumor cells.

In some embodiments according to any of the compositions described above, the composition, each of the tumor cells further comprise a second nucleic acid encoding a second reporter protein (e.g., luciferase or GFP), such as an intracellular fluorescent protein, e.g., GFP. In some embodiments, the expression of the second nucleic acid is also controlled by the inducible promoter (e.g., TetOn), i.e., both the nucleic acid encoding the reporter protein and the second nucleic acid encoding the second reporter protein are under the same promoter control. In some embodiments, the expression of the second nucleic acid is controlled by a second inducible promoter (e.g., TetOn). In some embodiments, the inducible promoter and the second inducible promoter are the same (e.g., both are TetOn promoters). In some embodiments, the inducible promoter and the second inducible promoter are different. In some embodiments, the second nucleic acid encoding the second reporter protein and the nucleic acid encoding the reporter protein are on the same vector, either under same promoter control, or under controls of different promoters. In some embodiments, the second nucleic acid encoding the second reporter protein and the nucleic acid encoding the reporter protein are on different vectors. In some embodiments, the two different vectors are transduced into the tumor cells simultaneously or sequentially. In some embodiments, the second reporter protein is the same as the report protein. In some embodiments, the second reporter protein (e.g., GFP) is different from the report protein (e.g., luciferase). In some embodiments, the second reporting protein is selected from the group consisting of luciferase, secreted alkaline phosphatase, and secreted fluorescent protein, or any combination thereof. In some embodiments, the second reporting protein is a luciferase selected from the group consisting of: Oplophorus luciferase, beetle luciferase, Renilla luciferase, Metridia luciferase, Gaussia luciferase, and NANOLUC luciferase, or any combination thereof.

In some embodiments according to any of the compositions described above, each of the tumor cells further comprises a third nucleic acid encoding an siRNA, a CRISPR/Cas, a ZFN, or a TALEN construct (“KO construct”) targeting an inhibitory checkpoint molecule (e.g., PD-L1) of the tumor cells. In some embodiments, the expression of the third nucleic acid is also controlled by the inducible promoter (e.g., TetOn), i.e., both the nucleic acid encoding the reporter protein and the third nucleic acid encoding the KO construct are under the same promoter control. In some embodiments, the expression of the third nucleic acid is controlled by a third inducible promoter (e.g., TetOn). In some embodiments, the inducible promoter and the third inducible promoter are the same (e.g., both are TetOn promoters). In some embodiments, the inducible promoter and the third inducible promoter are different. In some embodiments, the third nucleic acid encoding the KO construct and the nucleic acid encoding the reporter protein are on the same vector, either under same promoter control, or under controls of different promoters. In some embodiments, the third nucleic acid encoding the KO construct and the nucleic acid encoding the reporter protein are on different vectors. In some embodiments, the two different vectors are transduced into the tumor cells simultaneously or sequentially.

Further provided by the present invention are isolated nucleic acids that encode the reporter protein, vectors comprising such nucleic acids encoding the reporter protein under an inducible promoter (e.g., can further comprising a second nuclei acid encoding a second reporter protein and/or an KO construct on the same vector, under same or different promoter control), tumor cells comprising such vectors, and kits for conducting any of the methods described herein.

### BRIEF DESCRIPTION OF THE DRAWINGS

FIGS. 1A-1B depict exemplary methods of the present invention.

FIG. 2 depicts an exemplary molecular construct for the inducible expression of dual reporters eGFP and snLuc in the methods of the invention.

FIGS. 3A-3B demonstrate linear correlation between fluorescence intensity (EGFP) detected in a dual reporter tumor cell (breast cancer SK-BR-3) sample of the disclosure and the number of live tumor cells in the sample, when co-incubated with NK92 cells.

FIG. 4A demonstrates linear correlation between snLuciferase luminescence detected in samples of different dual reporter tumor cell types and the number of live tumor cells in the samples. Control samples were not contacted with the induction agent. FIG. 4B shows that induction increased expression of snLuciferase in various dual reporter tumor cells by about 50- to about 850-fold.

FIG. 5 depicts a dose response curve of Herceptin® mediated ADCC of NK92 cells on dual reporter SK-BR-3 cells generated from an exemplary cell killing assay.

FIG. 6A shows EGFP signal in dual reporter SK-BR-3 tumor cells in the presence of varying concentrations of Herceptin® antibody and fixed amount of NK92 cells (E:T ratio of 3:1). Bright filed pictures served as control for the experimental condition. FIG. 6B illustrates a dose-dependent relationship between dual reporter SK-BR-3 tumor cell survival and Herceptin® antibody concentration based on fluorescence and snLuciferase intensity.

FIGS. 7A-7D demonstrate effector cell killing effects under various antibody concentrations and tumor-to-effector cell ratios in various cancer cell lines using an exemplary method.

FIGS. 8A-8B demonstrate continuous real-time monitoring of an exemplary cell-killing method in various cancer cell lines under different effector-to-tumor cell ratios (1:1 or 5:1) and various concentrations of trispecific anti-HER2/anti-CD47/anti-CD3 antibody.

FIGS. 9A-9D demonstrate continuous monitoring of antibody-mediated effector cell (stimulated or unstimulated PBMC) killing on three-dimensional tumor spheroids formed with fibroblasts, using trispecific anti-HER2/anti-CD47/anti-CD3 antibody (FIGS. 9A-9B) or trispecific anti-PD-L1/anti-CD47/anti-CD3 antibody (FIGS. 9C-9D) under different concentrations.

FIG. 10 demonstrates an exemplary method for monitoring tumor cell (MDA-MB-231)-killing using multiple cell-killing immunomodulating agents (anti-PD-1 antibody and trispecific anti-HER2/anti-CD47/anti-CD3 antibody) in the presence of PBMCs.

FIG. 11 demonstrates continuous real-time monitoring of an exemplary tumor cell (MDA-MB-231)-killing method using multiple cell-killing immunomodulating agents (anti-PD-1 antibody and trispecific anti-HER2/anti-CD47/anti-CD3 antibody) in the presence of PBMCs.

FIGS. 12A-12B demonstrate varying the total reaction time can affect the dose-response curves generated from the methods. Total reaction time (antibody/tumor cell/effector cell incubation, dox-induction, snLuciferase measurement) is indicated on top of each panel of FIG. 12A.

FIGS. 13A-13B demonstrate that the levels of both snLuciferase and EGFP reporters correlate with live dual reporter tumor cells (LnCaP, MDA-MB-231, and MDA-MB-468) co-cultured with T cells. FIG. 13B shows both bright field and EGFP images.

FIG. 14 depicts trispecific anti-HER2/anti-CD47/anti-CD3 antibody-mediated PBMC killing of dual reporter MDA-MB-231 cells under different antibody concentrations, and different reporter induction time.

FIGS. 15A-15D depict bispecific anti-HER2/anti-CD3 antibody (FIG. 15A) and trispecific anti-HER2/anti-CD47/anti-CD3 antibody (FIG. 15B) mediated T-cell killing on various dual reporter tumor cells, which is correlated to tumor antigen expression levels (FIG. 15C).

FIGS. 16A-16D depict the effect of different E:T ratios on anti-HER2/anti-CD3 antibody-mediated effector cell killing on dual reporter MDA-MB-231 cells.

FIGS. 17A-17C depict that stimulated T-cells increased T-cell mediated killing on MDA-MB-468 cells (FIGS. 17B-17C), but not on MDA-MB-231 cells (FIG. 17A), when co-incubated with different concentrations of trispecific anti-HER2/anti-CD47/anti-CD3 antibody and different stimulated vs. non-stimulated T-cell contents.

FIGS. 18A-18C depict that modulating PD-1/PD-L1 blockade can affect effector cell-mediated tumor cell killing.

FIGS. 19A-19D depict anti-HER2 antibody trastuzumab (Herceptin®) mediated NK cell ADCC on dual reporter SK-BR-3 cells, which was affected by the timing of reporter protein induction by dox. FIG. 19A shows ADCC effect measured by snLuc signal. FIGS. 19B and 19D show ADCC effect measured by EGFP signal.

FIGS. 20A-20B depict tumor antigen (HER2) expression level affects trastuzumab (Herceptin®) mediated NK cell ADCC on various dual reporter tumor cells.

FIGS. 21A-21D depict the effect of different E:T ratios on anti-HER2 antibody trastuzumab-mediated ADCC on SK-BR-3 cells by unstimulated PBMCs.

FIGS. 22A-22B depict trastuzumab-mediated ADCC by NK92 cells on dual reporter SK-BR-3 cells can be detected in patient serum.

FIGS. 23A-23D depict trastuzumab-mediated ADCC by NK92 cells on 3D LnCaP spheroids. FIGS. 23A-23C depict EGFP signal measurement. FIGS. 23B-23D depict snLuciferase signal measurement.

### DETAILED DESCRIPTION OF THE INVENTION

The present application provides methods for evaluating the effectiveness of a cell-killing agent using tumor cells expressing a reporter protein, such as secreted reporter proteins. The assays described herein comprises two phases: 1) a cell killing phase and 2) an evaluating phase. During the cell killing phase, the cell-killing agents are brought into contact with the tumor cells and allowed to exert cell killing effect. During a subsequent evaluating phase, the amount of expressed reporter protein is determined, which negatively correlates with the effectiveness of the cell-killing agent. This method allows us to semi-quantify target cells' responses to the cell-killing agents.

In one exemplary method, the tumor cells comprise a nucleic acid encoding a reporter protein operatively linked to an inducible promoter. The method comprises two phases: a silent phase and an expression phase. In the silent phase, the tumor cells have been contacted with the cell-killing agent, but have not been induced to express the reporter protein. The silent phase ends when the tumor cells are induced to express the reporter protein. By determining the amount of reporter protein produced by the tumor cells, optionally by comparing with a control sample without the cell-killing agent, and/or optionally by comparing with a control sample contacted with the cell-killing agent but without reporter protein induction, the amount of cell-killing can be determined. Uncoupling the silent (cell-killing) and expression phases (cell killing can continue to happen in the expression phase) allows for a broad range of applications.

One advantage of having a silent phase is that there may be less background expression of the reporter protein. If the reporter protein is constitutively expressed, tumor cells that died as a result of the cell-killing agent could release the reporter protein into the media, thereby giving high background levels of reporter protein. Further, the continuous accumulation of secreted reporter proteins in the media over time could dampen the sensitivity and/or affect the accuracy of cytotoxicity assays. By having a silent phase with no expression followed by an expression phase, the methods herein provide greater sensitivity.

Furthermore, having a silent phase may provide a way for controlling the timing of detecting tumor killing, for optimizing assay conditions to achieve maximum cytotoxicity. For example, if cell killing is known to take many days, the expression phase can be delayed longer. If the cell killing is known to take a few hours, the expression phase can be started sooner after the contacting step (contacting tumor cells and cell-killing agent). The timing of the expression phase can vary based on the type of cell-killing agent used and/or the experimental conditions of the methods of the disclosure.

In some instances, the tumor cells comprise a nucleic acid encoding a secretable reporter protein that is operably linked to a constitutive promoter. The silent phase and expression phase are created by removing and replacing the media. Each round of removing and replacing media “resets” the amount of secretable reporter protein in the media and results in a new expression phase. For example, the silent phase can occur when a cell-killing agent is contacted with tumor cells constitutively expressing secretable reporter protein. The expression phase starts when the media is removed and replaced. In this new expression phase, the amount of secreted reporter protein can be determined, thereby determining the effectiveness of the cell-killing agent.

The timing of the silent phase can vary based on the cell killing agents (e.g., PBMCs, NK cells, T cells, CAR-T cells, therapeutic compounds, antibody-drug conjugates (ADCs), antibodies such as BiTE, etc.) and experimental conditions (e.g., 2D or 3D culture, effector:tumor (E:T) cell ratio, total cell number, tumor cell types, tumor antigen expression level, etc.). For example, NK cell-killing under 2D conditions is fast and typically occurs in hours. In contrast, T-cell killing in 3D spheroid conditions can last several days. During the expression phase, target cell viability/survival rate can be monitored through the expression level of the reporter proteins.

The methods described herein are particularly useful in a 3D spheroid tumor model, when tumor cells are admixed with other types of cells such as stromal cells (e.g., fibroblasts). 3D spheroids mimic the complex tumor microenvironment between cancer and stromal cells, but detection of cell killing is more challenging due to the complexity of the spheroid structure and the potential dilution effect of the non-tumor cells. In one embodiment of the present application, secreted reporter proteins are used, which further increases sensitivity of the assay.

Expressing the secretable reporter protein under an inducible system as described herein is advantageous, including, but are not limited to: 1) can be used to mimic immunosuppression observed in in vivo tumor microenvironment (e.g., immunosuppressive effect of PD-1/PD-L1 blockade), and study cytotoxicity of cell-killing agents, such as by knocking-out tumor cell PD-L1 expression or over-expressing PD-L1 on tumor cells; 2) can be used to sensitively and reproducibly detect cell killing by cell-killing agents in both 2D and 3D culture systems (e.g., spheroids) and/or co-culturing with other cell types to mimic cancer microenvironment; 3) can be used to study cytotoxicity induced by various cell-killing agents, such as different compounds, immune effector cells (e.g., engineered or non-engineered, such as CTLs, NKs, CAR-T, PBMCs, etc.), or immunotherapy candidates (such as immune checkpoint inhibitors, anti-tumor antigen antibody, multispecific antibody that targets effector cells to tumor cells), etc., on various target cell types (e.g., various cancer types), under various mechanisms of action (e.g., ADCC, nonspecific immune cell killing, multispecific antibody that targets effector cells to tumor cells); 3) provides sensitive, semi-quantitate assay system to study cell killing effects, for example, ADCC can be detected under low target antigen-expression level; 4) can be used for real-time continuous monitoring of cell killing effects over a period of time; 5) ADCC can be quantified and monitored using current invention in high concentrations of patient serums, which are often difficult to detect due to the low sensitivity of current ADCC assays—suggesting that the current system could serve as a useful tool to evaluate the potency of potential vaccines; 6) can be used to screen for new and/or improved compounds, engineered immune effector cells, or immunotherapy candidates in a sensitive and high-throughput manner; 7) by controlling total reaction time and when the reporter proteins are expressed from tumor cells, experimental conditions can be optimized and the timing when cytotoxicity is maximized can be selected, resulting in a highly sensitive and versatile assay; and 8) can be used to detect patient-to-patient variations on response to candidate therapeutic agents.

Thus, the present application in one aspect provides methods of evaluating the effectiveness of a cell-killing agent on a population of tumor cells, wherein each tumor cell comprises a nucleic acid encoding a reporter protein (e.g., luciferase or GFP). In another aspect, there are provided compositions comprising tumor cells comprising nucleic acid encoding a reporter protein (e.g., luciferase or GFP), which are useful for carrying out the cell killing assays described here. In some embodiments, the expression of the reporter protein is under control of an inducible promoter. Also provided are kits and articles of manufacture useful for carrying out the methods described herein.

### Definitions

As used herein, “antibody dependent cell-mediated cytotoxicity” or “ADCC” generally refers to a form of cytotoxicity in which secreted immunoglobulin (Ig) bound onto Fc receptors present on certain cytotoxic cells (e.g., NK cells, NKT cells, neutrophils, and macrophages) enables these cytotoxic effector cells to bind specifically to an antigen-bearing target cell. The effector cells can subsequently kill the target cell with cytotoxins. The ability of any particular antibody to mediate killing of the target cell by ADCC can be assayed. To assess ADCC activity an antibody of interest can be added to target cells in combination with immune effector cells, which may be activated by the antigen-antibody complexes, resulting in cytolysis of the target cell.

As used herein, a “cell-killing agent” generally refers to an agent that participates, directly and/or indirectly, in killing cells. Direct cell killing agents can be those that directly interact with the tumor cell in order to induce killing. Indirect cell killing agents are those which indirectly interact with the tumor cell in order to induce killing. The term “cell-killing agent” comprises both direct and indirect mechanisms of action for cell-killing. As such, a cell-killing agent such as a small molecule, an immune effector cell, an antibody, and/or an immunotherapy, can each be both a direct cell killing agent and an indirect cell-killing agent, depending on their mechanism of action. For example, a small molecule can directly kill a tumor cell by binding to receptors on the tumor cell or passing through the tumor cell membrane. A small molecule can also indirectly kill a tumor cell by acting as an allosteric modulator on another cell's receptors which would activate the immune cell for killing the tumor cell. As another example, an immunotherapy can bind to an immune cell and activate it for killing a tumor cell, whereby the immunotherapy does not directly bind to the tumor cell. In some instances, the immunotherapy can directly bind the tumor cell and kill it, such as via complement-dependent cytotoxicity (CDC), or via antibody-drug conjugate (ADC). In some instances, the immunotherapy can directly bind the tumor cell and indirectly kills the cell, due to its mechanism of action (e.g., such as interaction with an immune cell via the BiTE format). For example, in antibody-dependent cellular cytotoxicity (ADCC), an antibody binds to the target tumor cells via tumor antigen-binding domain, and the antibody Fc binds to FcR (e.g., CD16) on immune effector cells (e.g., NK cells, NKT cells), and target the immune effector cells to tumor site for killing. Macrophages, neutrophils, cosinophils can also effect ADCC. In antibody-dependent cellular phagocytosis (ADCP), an antibody can eliminate bound target cell via binding of its Fc domain to specific receptors on phagocytic cells, and eliciting phagocytosis. Monocytes, macrophages, neutrophils, and dendritic cells can mediate ADCP. A cell-killing agent can refer to any cell-killing agent alone and it can refer to any combination of cell-killing agents that, based on their mechanisms of action, kill cells. For example, a cell-killing agent can refer to an effector or immune cell alone, an antibody alone, a small molecule alone, or immunotherapy alone, or the term can refer the combination(s) of an effector or immune cell and an antibody, immunotherapy, or drug.

“Antibody effector functions” refer to those biological activities attributable to the Fc region (a native sequence Fc region or amino acid sequence variant Fc region) of an antibody, and vary with the antibody isotype. Examples of antibody effector functions include: C1q binding and complement dependent cytotoxicity: Fc receptor binding: antibody-dependent cell-mediated cytotoxicity (ADCC); phagocytosis; down regulation of cell surface receptors (e.g., B cell receptors); and B cell activation. “Reduced or minimized” antibody effector function means that which is reduced by at least 50% (alternatively 60%, 65%, 70%, 75%, 80%, 85%, 90%, 95%, 96%, 97%, 98%, or 99%) from the wild type or unmodified antibody. The determination of antibody effector function is readily determinable and measurable by one of ordinary skill in the art. In a preferred embodiment, the antibody effector functions of complement binding, complement dependent cytotoxicity and antibody dependent cytotoxicity are affected. In some embodiments, effector function is eliminated through a mutation in the constant region that eliminated glycosylation, e.g., “effectorless mutation.” In some embodiments, the effectorless mutation is an N297A or DANA mutation (D265A+N297A) in the C_{H}2 region. Shields et al., J. Biol. Chem. 276 (9): 6591-6604 (2001). Alternatively, additional mutations resulting in reduced or eliminated effector function include: K322A and L234A/L235A (LALA). Alternatively, effector function can be reduced or eliminated through production techniques, such as expression in host cells that do not glycosylate (e.g., E. coli.) or in which result in an altered glycosylation pattern that is ineffective or less effective at promoting effector function (e.g., Shinkawa et al., J. Biol. Chem. 278(5): 3466-3473 (2003).

“Antibody-dependent cell-mediated cytotoxicity” or ADCC refers to a form of cytotoxicity in which secreted Ig bound onto Fc receptors (FcRs) present on certain cytotoxic cells (e.g., natural killer (NK) cells, neutrophils and macrophages) enable these cytotoxic effector cells to bind specifically to an antigen-bearing target cell and subsequently kill the target cell with cytotoxins. The antibodies “arm” the cytotoxic cells and are required for killing of the target cell by this mechanism. The primary cells for mediating ADCC, NK cells, express FcγRIII only, whereas monocytes express FcγRI, FcγRII, and FcγRIII. Fc expression on hematopoietic cells is summarized in Table 2 on page 464 of Ravetch and Kinet, Annu. Rev. Immunol. 9: 457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and natural killer (NK) cells. Alternatively, or additionally, ADCC activity of the molecule of interest may be assessed in vivo, e.g., in an animal model such as that disclosed in Clynes et al., PNAS USA 95:652-656 (1998).

The term “Fc region,” “fragment crystallizable region,” or “Fc domain” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. Suitable native-sequence Fc regions for use in the antibodies described herein include human IgG1, IgG2 (IgG2A. IgG2B), IgG3 and IgG4.

“Fc receptor” or “FcR” describes a receptor that binds the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors, FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (See M. Daëron, Annu. Rev. Immunol. 15:203-234 (1997). FcRs are reviewed in Ravetch and Kinet, Annu. Rev. Immunol. 9: 457-92 (1991); Capel et al., Immunomethods 4: 25-34 (1994); and de Haas et al., J. Lab. Clin. Med. 126: 330-41 (1995). Other FcRs, including those to be identified in the future, are encompassed by the term “FcR” herein.

The term “Fc receptor” or “FcR” also includes the neonatal receptor, FcRn, which is responsible for the transfer of maternal IgGs to the fetus. Guyer et al., J. Immunol. 117: 587 (1976) and Kim et al.. J. Immunol. 24: 249 (1994). Methods of measuring binding to FcRn are known (see, e.g., Ghetie and Ward, Immunol. Today 18: (12): 592-8 (1997): Ghetie et al., Nature Biotechnology 15 (7): 637-40 (1997); Hinton et al., J. Biol. Chem. 279 (8): 6213-6 (2004): WO 2004/92219 (Hinton et al.). Binding to FcRn in vivo and serum half-life of human FcRn high-affinity binding polypeptides can be assayed, e.g., in transgenic mice or transfected human cell lines expressing human FcRn, or in primates to which the polypeptides having a variant Fc region are administered. WO 2004/42072 (Presta) describes antibody variants which improved or diminished binding to FcRs. See also, e.g., Shields et al., J. Biol. Chem. 9(2): 6591-6604 (2001).

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass) which are bound to their cognate antigen. To assess complement activation, a CDC assay, e.g., as described in Gazzano-Santoro et al., J. Immunol. Methods 202: 163 (1996), may be performed. Antibody variants with altered Fc region amino acid sequences and increased or decreased C1q binding capability are described in U.S. Pat. No. 6,194,551B1 and WO99/51642. The contents of those patent publications are specifically incorporated herein by reference. See, also, Idusogie et al. J. Immunol. 164: 4178-4184 (2000).

Half maximal inhibitory concentration (IC₅₀) is a measure of the effectiveness of a substance (such as an antibody) in inhibiting a specific biological or biochemical function. It indicates how much of a particular drug or other substance (inhibitor, such as an antibody) is needed to inhibit a given biological process by half. The values are typically expressed as molar concentration. IC₅₀ is comparable to an “EC₅₀” for agonist drug or other substance (such as an antibody or a cytokine). EC₅₀ also represents the plasma concentration required for obtaining 50% of a maximum effect in vivo. As used herein, an “IC₅₀” is used to indicate the effective concentration of an antibody needed to neutralize 50% of the antigen bioactivity in vitro. IC₅₀ or EC₅₀ can be measured by bioassays such as inhibition of ligand binding by FACS analysis (competition binding assay), cell based cytokine release assay, or amplified luminescent proximity homogeneous assay (AlphaLISA).

As used herein, a “tumor cell,” used either in the singular or plural form, generally refers to cells that have undergone a malignant transformation that makes them pathological to the host organism. Primary cancer cells can be readily distinguished from non-cancerous cells by techniques such as histological examination. A tumor cell can refer to a primary cancer cell, and any cell derived from a tumor cell ancestor, including metastasized tumor cells, and in vitro cultures and cell lines derived from tumor cells.

The term “antibody” or “antibody moiety” is used in the broadest sense and encompasses various antibody structures, including but not limited to monoclonal antibodies, multispecific antibodies (e.g., bispecific antibodies), full-length antibodies and antigen-binding fragments thereof, so long as they exhibit the desired antigen-binding activity. An antibody can be chimeric, humanized, human antibody, or antibody of non-human source (e.g., mouse Ab).

The basic 4-chain antibody unit is a heterotetrameric glycoprotein composed of two identical light (L) chains and two identical heavy (H) chains. An IgM antibody consists of 5 of the basic heterotetramer units along with an additional polypeptide called a J chain, and contains 10 antigen-binding sites, while IgA antibodies comprise from 2-5 of the basic 4-chain units which can polymerize to form polyvalent assemblages in combination with the J chain. In the case of IgGs, the 4-chain unit is generally about 150,000 Daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (V_{H}) followed by three constant domains (C_{H}) for each of the α and γ chains and four C_{H} domains for ρ and ε isotypes. Each L chain has at the N-terminus, a variable domain (V_{L}) followed by a constant domain at its other end. The V_{L} is aligned with the V_{H} and the C_{L} is aligned with the first constant domain of the heavy chain (C_{H}1). Particular amino acid residues are believed to form an interface between the light chain and heavy chain variable domains. The pairing of a V_{H} and V_{L} together forms a single antigen-binding site. The L chain from any vertebrate species can be assigned to one of two distinct types, called kappa and lambda, based on the amino acid sequences of their constant domains. Depending on the amino acid sequence of the constant domain of their heavy chains (C_{H}), immunoglobulins can be assigned to different classes or isotypes. There are five classes of immunoglobulins: IgA, IgD, IgE, IgG and IgM, having heavy chains designated α, δ, ε, γ and μ, respectively. The γ and α classes are further divided into subclasses on the basis of relatively minor differences in the C_{H} sequence and function, e.g., humans express the following subclasses: IgG1, IgG2A, IgG2B, IgG3, IgG4, IgA1 and IgA2.

An “antibody fragment” or “antigen-binding fragment” comprises a portion of an intact antibody, preferably the antigen binding and/or the variable region of the intact antibody. Examples of antibody fragments include, but are not limited to Fab, Fab′, F(ab′)₂ and Fv fragments; diabodies; linear antibodies (see U.S. Pat. No. 5,641,870, Example 2; Zapata et al., Protein Eng. 8(10): 1057-1062(1995)); single-chain antibody (scFv) molecules; single-domain antibodies (such as V_{H}H), and multispecific antibodies formed from antibody fragments. Papain digestion of antibodies produced two identical antigen-binding fragments, called “Fab” fragments, and a residual “Fc” fragment, a designation reflecting the ability to crystallize readily. The Fab fragment consists of an entire L chain along with the variable domain of the H chain (V_{H}), and the first constant domain of one heavy chain (C_{H}1). Each Fab fragment is monovalent with respect to antigen binding, i.e., it has a single antigen-binding site. Pepsin treatment of an antibody yields a single large F(ab′)₂ fragment which roughly corresponds to two disulfide linked Fab fragments having different antigen-binding activity and is still capable of cross-linking antigen. Fab′ fragments differ from Fab fragments by having a few additional residues at the carboxy-terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

As used herein, the term “specifically binds,” “specifically recognizes.” or is “specific for” refers to measurable and reproducible interactions such as binding between a target and an antigen binding protein, which is determinative of the presence of the target in the presence of a heterogeneous population of molecules including biological molecules. For example, an antigen binding protein that specifically binds a target is an antigen binding protein that binds this target with greater affinity, avidity, more readily, and/or with greater duration than it binds other targets. In some embodiments, the extent of binding of an antigen binding protein to an unrelated target is less than about 10% of the binding of the antigen binding protein to the target as measured, e.g., by a radioimmunoassay (RIA). In some embodiments, an antigen binding protein that specifically binds a target has a dissociation constant (K_{D}) of ≤10⁻⁵ M, ≤10⁻⁶ M, ≤10⁻⁷ M, ≤10⁻⁸ M, ≤10⁻⁹ M, ≤10⁻¹⁰ M, ≤10⁻¹¹ M, or ≤10⁻¹² M. In some embodiments, an antigen binding protein specifically binds an epitope on a protein that is conserved among the protein from different species. In some embodiments, specific binding can include, but does not require exclusive binding. Binding specificity of the antibody or antigen-binding domain can be determined experimentally by methods known in the art. Such methods comprise, but are not limited to Western blots, ELISA-, RIA-, ECL-, IRMA-, EIA-, BIACORE™-tests and peptide scans.

The term “specificity” refers to selective recognition of an antigen binding protein for a particular epitope of an antigen. Natural antibodies, for example, are monospecific. The term “multispecific” as used herein denotes that an antigen binding protein has polyepitopic specificity (i.e., is capable of specifically binding to two, three, or more, different epitopes on one biological molecule or is capable of specifically binding to epitopes on two, three, or more, different biological molecules). “Bispecific” as used herein denotes that an antigen binding protein has two different antigen-binding specificities. Unless otherwise indicated, the order in which the antigens bound by a bispecific antibody listed is arbitrary. That is, for example, the terms “anti-CD3/HER2,” “anti-HER2/CD3,” “CD3×HER2” and “HER2×CD3” may be used interchangeably to refer to bispecific antibodies that specifically bind to both CD3 and HER2. The term “monospecific” as used herein denotes an antigen binding protein that has one or more binding sites each of which bind the same epitope of the same antigen.

The term “valent” as used herein denotes the presence of a specified number of binding sites in an antigen binding protein. A natural antibody for example or a full-length antibody has two binding sites and is bivalent. As such, the terms “trivalent”, “tetravalent”, “pentavalent” and “hexavalent” denote the presence of two binding site, three binding sites, four binding sites, five binding sites, and six binding sites, respectively, in an antigen binding protein.

An “isolated” nucleic acid molecule encoding a construct, antibody, or antigen-binding fragment thereof described herein is a nucleic acid molecule that is identified and separated from at least one contaminant nucleic acid molecule with which it is ordinarily associated in the environment in which it was produced. Preferably, the isolated nucleic acid is free of association with all components associated with the production environment. The isolated nucleic acid molecules encoding the polypeptides described herein is in a form other than in the form or setting in which it is found in nature. Isolated nucleic acid molecules therefore are distinguished from nucleic acid encoding the polypeptides and antibodies described herein existing naturally in cells. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

The term “vector,” as used herein, generally refers to a nucleic acid molecule capable of propagating another nucleic acid to which it is linked. The term can include the vector as a self-replicating nucleic acid structure as well as the vector incorporated into the genome of a tumor cell into which it has been introduced. Certain vectors are capable of directing the expression of nucleic acids to which they are operatively linked. Such vectors are referred to herein as “expression vectors.”

The term “transfected” or “transformed” or “transduced” as used herein refers to a process by which exogenous nucleic acid is transferred or introduced into the tumor cell. A “transfected” or “transformed” or “transduced” cell is one which has been transfected, transformed or transduced with exogenous nucleic acid. The cell includes the primary subject cell and its progeny.

The terms “host cell,” “host cell line,” and “host cell culture” are used interchangeably and refer to cells into which exogenous nucleic acid has been introduced, including the progeny of such cells. Host cells include “transformants” and “transformed cells.” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

As used herein, the singular forms “a”, “an”, and “the” include plural referents unless the context clearly dictates otherwise.

It is understood that embodiments of the invention described herein include “consisting” and/or “consisting essentially of” embodiments.

Reference to “about” a value or parameter herein includes (and describes) variations that are directed to that value or parameter per se. For example, description referring to “about X” includes description of “X”.

The term “about X-Y” used herein has the same meaning as “about X to about Y.”

As used herein, reference to “not” a value or parameter generally means and describes “other than” a value or parameter. For example, the method is not used to treat cancer of type X means the method is used to treat cancer of types other than X.

All publications, patents, and patent applications mentioned in this specification are herein incorporated by reference to the same extent as if each individual publication, patent, or patent application was specifically and individually indicated to be incorporated by reference.

As will be apparent to those of skill in the art upon reading this disclosure, each of the individual embodiments described and illustrated herein has discrete components and features which may be readily separated from or combined with the features of any of the other several embodiments without departing from the scope or spirit of the methods, compositions and kits of the disclosure. Any recited method can be carried out in the order of events recited or in any other order which is logically possible.

### Methods of the Present Application

In one aspect, the disclosure provides for a method of evaluating the effectiveness of a cell-killing agent (e.g., small compound, immune effector cell, antibody such as multispecific antibody. ADC, immunomodulator such as immune checkpoint inhibitor, etc., or any combinations thereof) on a population of tumor cells, the method comprising: contacting the tumor cells with a cell-killing agent, wherein each of the tumor cells comprises a nucleic acid encoding a reporter protein (e.g., GFP or luciferase); allowing expression of the nucleic acid to produce the reporter protein; and determining the amount of the reporter protein, wherein the amount of the reporter protein negatively correlates with the effectiveness of the cell killing agent. In some embodiments, the contacting step is carried out at a cell-killing phase, and the determining step is carried out at a subsequent evaluating phase. In some embodiments, the reporter protein is secreted by the tumor cells. In some embodiments, the contacting step occurs for at least about 24 hours prior to detection. In some embodiments, the contacting step occurs for about 4 to about 48 hours (such as about 24 to about 48 hours) prior to detection. In some embodiments, the contacting step occurs for up to about 6 days (e.g., about any of 1, 2, 3, 4, 5, or 6 days). In some embodiments, each of the tumor cells further comprises a second nucleic acid encoding a second reporter protein (e.g., GFP or luciferase).

In some embodiments, there is provided a method of evaluating the effectiveness of a cell-killing agent (e.g., small compound, immune effector cell, antibody such as multispecific antibody, ADC, immunomodulator such as immune checkpoint inhibitor, etc., or any combinations thereof) on a population of tumor cells, comprising: a) contacting the tumor cells with a cell-killing agent, wherein each of the tumor cells comprises a nucleic acid encoding a reporter protein (.g., GFP or luciferase), wherein the expression of the nucleic acid is controlled by an inducible promoter (e.g., TetOn), b) inducing expression of the nucleic acid to produce the reporter protein, and c) determining the amount of the reporter protein, wherein the amount of the reporter protein negatively correlates with the effectiveness of the cell killing agent. In some embodiments, the contacting step occurs simultaneously with the inducing step. In some embodiments, there is provided a method of evaluating the effectiveness of a cell-killing agent (e.g., small compound, immune effector cell, antibody such as multispecific antibody, ADC, immunomodulator such as immune checkpoint inhibitor, etc., or any combinations thereof) on a population of tumor cells, comprising: a) contacting the tumor cells with a cell-killing agent, wherein each of the tumor cells comprises a nucleic acid encoding a reporter protein (.g., GFP or luciferase), wherein expression of the nucleic acid is controlled by an inducible promoter (e.g., TetOn), b) inducing expression of the nucleic acid to produce the reporter protein, and c) determining the amount of the reporter protein, wherein the amount of the reporter protein negatively correlates with the effectiveness of the cell killing agent, wherein the contacting step is carried out at a cell-killing phase, and wherein the determining step is carried out at a subsequent evaluating phase. In some embodiments, the reporter protein is secreted by the tumor cells. In some embodiments, the contacting step occurs for at least about 24 hours prior to the inducing step. In some embodiments, the contacting step occurs for about 4 to about 48 hours (such as about 24 to about 48 hours) prior to the inducing step. In some embodiments, the contacting step occurs for up to about 6 days (e.g., about any of 1, 2, 3, 4, 5, or 6 days) prior to the inducing step. In some embodiments, the inducing step occurs for about 4 to about 48 hours (e.g., about 4 to about 8 hours, or about 24 to about 48 hours). In some embodiments, the inducing step comprises treating the tumor cells with an induction agent (e.g., tetracycline, doxycycline, estrogen receptor, and cumate, or any combination thereof). In some embodiments, the reporter protein is selected from the group consisting of luciferase, secreted alkaline phosphatase, and secreted fluorescent protein, or any combination thereof. In some embodiments, the luciferase is selected from the group consisting of Oplophorus luciferase, beetle luciferase, Renilla luciferase, Metridia luciferase, Gaussia luciferase, and NANOLUC luciferase, or any combination thereof. In some embodiments, the determining step comprises detecting the reporter protein over different time points. In some embodiments, the tumor cells are present in a mixture comprising a second population of cells (e.g., fibroblast cells, stromal cells, endothelial cells, tumor associated macrophages, myeloid-derived suppressive cells, or any combination/variant thereof, or any combination thereof). In some embodiments, the tumor cells are present in a 3D spheroid or a 2D monolayer. In some embodiments, the cell-killing agent is selected from the group consisting of a cytotoxin, a drug, a small molecule, and a small molecule compound, or any combination thereof. In some embodiments, the cell-killing agent is an immune cell. In some embodiments, the cell-killing agent is an immunomodulating agent, and wherein the contacting step is conducted in the presence of an immune cell. In some embodiments, the immune cell is selected from the group consisting of a natural killer (NK) cell, a natural killer T (NKT) cell, a T cell, a CAR-T cell, a CD14+ cell, a dendritic cell, and a PBMC cell, or any combination thereof. In some embodiments, the immunomodulating agent is an immune checkpoint inhibitor (e.g., inhibits PD-1, PD-L1, PD-L2, Siglec. BTLA, CTLA-4, or any combination thereof). In some embodiments, the cell-killing agent is an antibody (e.g., a PD-1 antibody, an anti-PD-L1 antibody, an anti-CD47 antibody, an anti-HER2 antibody, an anti-CD20 antibody, and an anti-CD3 antibody, or any combination thereof). In some embodiments, the antibody is multispecific (e.g., an anti-HER2/anti-CD3 antibody, an anti-HER2/anti-CD47/anti-CD3 antibody, or an anti-PD-L1/anti-CD47/anti-CD3 antibody). In some embodiments, the method further comprises contacting the tumor cells with a second cell-killing agent. In some embodiments, the second cell-killing agent (e.g., antibody) inhibits an inhibitory checkpoint molecule selected from the group consisting of PD-1, PD-L1, PD-L2. Siglec, BTLA, and CTLA-4, or any combination thereof. In some embodiments, the second cell-killing agent is an anti-PD-1 antibody or an anti-PD-L1 antibody. In some embodiments, the second cell-killing agent is an siRNA or a CRISPR/Cas construct targeting an inhibitory checkpoint molecule (e.g., PD-L1). In some embodiments, the contacting of the second cell-killing agent occurs simultaneously with the contacting of the cell-killing agent. In some embodiments, the nucleic acid encoding the reporter protein is introduced into the tumor cells by a retroviral or lentiviral vector system. In some embodiments, each of the tumor cells further comprises a second nucleic acid encoding a second reporter protein (e.g., luciferase or GFP). In some embodiments, the expression of the second nucleic acid is controlled by a second inducible promoter (e.g., TetOn). In some embodiments, the expression of the second nucleic acid is controlled by the same inducible promoter.

In some embodiments, there is provided a method of evaluating the effectiveness of a cell-killing agent (e.g., small compound, immune effector cell, antibody such as multispecific antibody, ADC, immunomodulator such as immune checkpoint inhibitor, etc., or any combinations thereof) on a population of tumor cells comprising a) contacting the tumor cells with a cell-killing agent, wherein each of the tumor cells comprises a nucleic acid encoding a reporter protein (e.g., luciferase or GFP), wherein expression of the nucleic acid is controlled by an inducible promoter (e.g., TetOn), b) inducing expression of the nucleic acid to produce the reporter protein, wherein the contacting step occurs before (e.g., about 4 to about 48 hours, or about 24 to about 48 hours before) the inducing step, and c) determining the amount of the reporter protein, wherein the amount of the reporter protein negatively correlates with the effectiveness of the cell killing agent. In some embodiments, the reporter protein is secreted by the tumor cells, wherein the contacting step is carried out at a cell-killing phase, and wherein the determining step is carried out at a subsequent evaluating phase. In some embodiments, the reporter protein is a secretable luciferase. In some embodiments, the determining step comprises detecting the reporter protein over different time points. In some embodiments, the tumor cells are present in a 3D spheroid with a second population of cells. In some embodiments, the second population of cells are fibroblast cells or stromal cells. In some embodiments, each of the tumor cells further comprises a second nucleic acid encoding a second reporter protein (e.g., luciferase or GFP). In some embodiments, the expression of the second nucleic acid is controlled by a second inducible promoter (e.g., TetOn). In some embodiments, the expression of the second nucleic acid is controlled by the same inducible promoter in a same vector. In some embodiments, each of the tumor cells further comprises a third nucleic acid encoding a CRISPR/Cas targeting an inhibitory checkpoint molecule (e.g., PD-L1).

In some embodiments, the inducing step can be carried out simultaneously with the contacting step. Due to the delay of protein expression upon induction, the reporter protein can be accurately determined during the subsequent evaluating step. Thus, in some embodiments, there is provided a method of evaluating the effectiveness of a cell-killing agent (e.g., small compound, immune effector cell, antibody such as multispecific antibody, ADC, immunomodulator such as immune checkpoint inhibitor, etc., or any combinations thereof) on a population of tumor cells comprising a) contacting the tumor cells with a cell-killing agent, wherein each of the tumor cells comprises a nucleic acid encoding a reporter protein (e.g., luciferase or GFP), wherein expression of the nucleic acid is controlled by an inducible promoter (e.g., TetOn), b) inducing expression of the nucleic acid to produce the reporter protein, wherein the contacting step occurs simultaneously with the inducing step, and c) determining the amount of the reporter protein, wherein the amount of the reporter protein negatively correlates with the effectiveness of the cell killing agent, wherein the contacting step is carried out at a cell-killing phase, and wherein the determining step is carried out at a subsequent evaluating phase. In some embodiments, the reporter protein is a secretable luciferase. In some embodiments, the determining step comprises detecting the reporter protein over different time points. In some embodiments, the tumor cells are present in a 3D spheroid with a second population of cells. In some embodiments, the second population of cells are fibroblast cells or stromal cells. In some embodiments, each of the tumor cells further comprises a second nucleic acid encoding a second reporter protein (e.g., luciferase or GFP). In some embodiments, the expression of the second nucleic acid is controlled by a second inducible promoter (e.g., TetOn). In some embodiments, the expression of the second nucleic acid is controlled by the same inducible promoter in a same vector. In some embodiments, each of the tumor cells further comprises a third nucleic acid encoding a CRISPR/Cas targeting an inhibitory checkpoint molecule (e.g., PD-L1).

In one aspect the disclosure provides for a method of evaluating the effectiveness of a cell-killing agent (e.g., small compound, immune effector cell, antibody such as multispecific antibody, ADC, immunomodulator such as immune checkpoint inhibitor, etc., or any combinations thereof) on a population of tumor cells comprising a) contacting the tumor cells with a cell-killing agent, where the tumor cells are present in a 3D spheroid, wherein each of the tumor cells comprises a nucleic acid encoding a reporter protein (e.g., luciferase or GFP), wherein expression of the nucleic acid is controlled by an inducible promoter (e.g., TetOn), b) inducing expression of the nucleic acid to produce the reporter protein, and c) determining the amount of the reporter protein, wherein the amount of the reporter protein negatively correlates with the effectiveness of the cell killing agent, wherein the contacting step is carried out at a cell-killing phase, and wherein the determining step is carried out at a subsequent evaluating phase. In some embodiments, the contacting step occurs before (e.g., about 4 to about 48 hours, or about 24 to about 48 hours before) the inducing step. In some embodiments, the contacting step occurs simultaneously with the inducing step. In some embodiments, the reporter protein is secreted by the tumor cells. In some embodiments, the reporter protein is a secretable luciferase. In some embodiments, the determining step comprises detecting the reporter protein over different time points. In some embodiments, the tumor cells are present in a 3D spheroid with a second population of cells. In some embodiments, the second population of cells are fibroblast cells or stromal cells. In some embodiments, each of the tumor cells further comprises a second nucleic acid encoding a second reporter protein (e.g., luciferase or GFP). In some embodiments, the expression of the second nucleic acid is controlled by a second inducible promoter (e.g., TetOn). In some embodiments, the expression of the second nucleic acid is controlled by the same inducible promoter in a same vector. In some embodiments, each of the tumor cells further comprises a third nucleic acid encoding a CRISPR/Cas targeting an inhibitory checkpoint molecule (e.g., PD-L1).

In one aspect the disclosure provides for a method of evaluating the effectiveness of a cell-killing agent on a population of tumor cells comprising a) contacting the tumor cells with a cell-killing agent, where the tumor cells are present in a 3D spheroid, wherein the cell-killing agent comprises a cytotoxin, drug, small molecule, and/or small molecule compound, wherein each of the tumor cells comprises a nucleic acid encoding a reporter protein (e.g., luciferase or GFP), wherein expression of the nucleic acid is controlled by an inducible promoter (e.g., TetOn), b) inducing expression of the nucleic acid to produce the reporter protein, and c) determining the amount of the reporter protein, wherein the amount of the reporter protein negatively correlates with the effectiveness of the cell killing agent, wherein the contacting step is carried out at a cell-killing phase, and wherein the determining step is carried out at a subsequent evaluating phase. In some embodiments, the reporter protein is secreted by the tumor cells. In some embodiments, the contacting step occurs before (e.g., about 4 to about 48 hours, or about 24 to about 48 hours before) the inducing step. In some embodiments, the contacting step occurs simultaneously with the inducing step. In some embodiments, the reporter protein is a secretable luciferase. In some embodiments, the determining step comprises detecting the reporter protein over different time points. In some embodiments, the tumor cells are present in a 3D spheroid with a second population of cells. In some embodiments, the second population of cells are fibroblast cells or stromal cells. In some embodiments, each of the tumor cells further comprises a second nucleic acid encoding a second reporter protein (e.g., luciferase or GFP). In some embodiments, the expression of the second nucleic acid is controlled by a second inducible promoter (e.g., TetOn). In some embodiments, the expression of the second nucleic acid is controlled by the same inducible promoter in a same vector. In some embodiments, each of the tumor cells further comprises a third nucleic acid encoding a CRISPR/Cas targeting an inhibitory checkpoint molecule (e.g., PD-L1). In some embodiments, the method further comprises contacting the tumor cells with a second cell-killing agent (e.g., small compound, immune effector cell, antibody such as multispecific antibody, ADC, immunomodulator such as immune checkpoint inhibitor, etc., or any combinations thereof). In some embodiments, the second cell-killing agent is an immune check point inhibitor (e.g., antibody) inhibiting PD-1, PD-L1, PD-L2, Siglec, BTLA, and/or CTLA-4. In some embodiments, the second cell-killing agent is an antibody specifically targeting the tumor cell and an immune cell (e.g., BiTE).

In one aspect the disclosure provides for a method of evaluating the effectiveness of a cell-killing agent on a population of tumor cells comprising a) contacting the tumor cells with a cell-killing agent, where the tumor cells are present in a 3D spheroid, wherein the cell-killing agent comprises an immune cell (e.g., NK, CTL, or PBMC), wherein each of the tumor cells comprises a nucleic acid encoding a reporter protein (e.g., luciferase or GFP), wherein expression of the nucleic acid is controlled by an inducible promoter (e.g., TetOn), b) inducing expression of the nucleic acid to produce the reporter protein, and c) determining the amount of the reporter protein, wherein the amount of the reporter protein negatively correlates with the effectiveness of the cell killing agent, wherein the contacting step is carried out at a cell-killing phase, and wherein the determining step is carried out at a subsequent evaluating phase. In some embodiments, the contacting step occurs before (e.g., about 4 to about 48 hours, or about 24 to about 48 hours before) the inducing step. In some embodiments, the contacting step occurs simultaneously with the inducing step. In some embodiments, the immune cell is an NK cell, an NKT cell, a T cell, a CAR-T cell, a CD14+ cell, a dendritic cell, and/or a PBMC cell. In some embodiments, the reporter protein is secreted by the tumor cells. In some embodiments, the reporter protein is a secretable luciferase. In some embodiments, the determining step comprises detecting the reporter protein over different time points. In some embodiments, the tumor cells are present in a 3D spheroid with a second population of cells. In some embodiments, the second population of cells are fibroblast cells or stromal cells. In some embodiments, each of the tumor cells further comprises a second nucleic acid encoding a second reporter protein (e.g., luciferase or GFP). In some embodiments, the expression of the second nucleic acid is controlled by a second inducible promoter (e.g., TetOn). In some embodiments, the expression of the second nucleic acid is controlled by the same inducible promoter in a same vector. In some embodiments, each of the tumor cells further comprises a third nucleic acid encoding a CRISPR/Cas targeting an inhibitory checkpoint molecule (e.g., PD-L1). In some embodiments, the method further comprises contacting the tumor cells with a second cell-killing agent (e.g., small compound, immune effector cell, antibody such as multispecific antibody, ADC, immunomodulator such as immune checkpoint inhibitor, etc., or any combinations thereof). In some embodiments, the second cell-killing agent is an immune check point inhibitor (e.g., antibody) inhibiting PD-1, PD-L. PD-L2, Siglec, BTLA, and/or CTLA-4. In some embodiments, the second cell-killing agent is an antibody specifically targeting the tumor cell and the immune cell (e.g., BiTE).

In one aspect the disclosure provides for a method of evaluating the effectiveness of a cell-killing agent on a population of tumor cells comprising a) contacting the tumor cells with a cell-killing agent, where the tumor cells are present in a 3D spheroid, wherein the cell-killing agent comprises an immune cell and an antibody, wherein each of the tumor cells comprises a nucleic acid encoding a reporter protein (e.g., luciferase or GFP), wherein expression of the nucleic acid is controlled by an inducible promoter (e.g., TetOn), b) inducing expression of the nucleic acid to produce the reporter protein, and c) determining the amount of the reporter protein, wherein the amount of the reporter protein negatively correlates with the effectiveness of the cell killing agent, wherein the contacting step is carried out at a cell-killing phase, and wherein the determining step is carried out at a subsequent evaluating phase. In some embodiments, the contacting step occurs before (e.g., about 4 to about 48 hours, or about 24 to about 48 hours before) the inducing step. In some embodiments, the contacting step occurs simultaneously with the inducing step. In some embodiments, the immune cell is an NK cell, an NKT cell, a T cell, a CAR-T cell, a CD14+ cell, a dendritic cell, and/or a PBMC cell. In some embodiments, the antibody is an immunomodulating agent and the contacting step is conducted in the presence of immune cells. In some embodiments, the antibody is an immune checkpoint inhibitor. In some embodiments, the immune checkpoint inhibitor inhibits PD-1, PD-L1, PD-L2, Siglec, BTLA, and/or CTLA-4. In some embodiments, the antibody is selected from the group consisting of an anti-PD-1 antibody (e.g., nivolumab such as Opdivo®), an anti-PD-L1 antibody, an anti-CD47 antibody, an anti-HER2 antibody (e.g., Trastuzmab such as Herceptin®), an anti-CD20 antibody, and an anti-CD3 antibody, or any combination thereof. In some embodiments, the antibody specifically recognizes both immune cells and tumor cells. In some embodiments, the antibody is an anti-HER2/anti-CD3 antibody, an anti-HER2/anti-CD47/anti-CD3 antibody, or an anti-PD-L1/anti-CD47/anti-CD3 antibody. In some embodiments, the reporter protein is secreted by the tumor cells. In some embodiments, the reporter protein is a secretable luciferase. In some embodiments, the determining step comprises detecting the reporter protein over different time points. In some embodiments, the tumor cells are present in a 3D spheroid with a second population of cells. In some embodiments, the second population of cells are fibroblast cells or stromal cells. In some embodiments, each of the tumor cells further comprises a second nucleic acid encoding a second reporter protein (e.g., luciferase or GFP). In some embodiments, the expression of the second nucleic acid is controlled by a second inducible promoter (e.g., TetOn). In some embodiments, the expression of the second nucleic acid is controlled by the same inducible promoter in a same vector. In some embodiments, each of the tumor cells further comprises a third nucleic acid encoding a CRISPR/Cas targeting an inhibitory checkpoint molecule (e.g., PD-L1). In some embodiments, the method further comprises contacting the tumor cells with a second cell-killing agent (e.g., small compound, immune effector cell, antibody such as multispecific antibody, ADC, immunomodulator such as immune checkpoint inhibitor, etc., or any combinations thereof). In some embodiments, the second cell-killing agent is an immune check point inhibitor (e.g., antibody) inhibiting PD-1, PD-L1, PD-L2, Siglec, BTLA, and/or CTLA-4, such as anti-PD-1 antibody.

In one aspect the disclosure provides for a method of evaluating the effectiveness of a cell-killing agent on a population of tumor cells comprising a) contacting the tumor cells with a cell-killing agent, where the tumor cells are present in a 3D spheroid, wherein the cell-killing agent comprises an antibody, wherein each of the tumor cells comprises a nucleic acid encoding a reporter protein (e.g., luciferase or GFP), wherein expression of the nucleic acid is controlled by an inducible promoter (e.g., TetOn), b) inducing expression of the nucleic acid to produce the reporter protein, and c) determining the amount of the reporter protein, wherein the amount of the reporter protein negatively correlates with the effectiveness of the cell killing agent, wherein the contacting step is carried out at a cell-killing phase, and wherein the determining step is carried out at a subsequent evaluating phase. In some embodiments, the contacting step occurs before (e.g., about 4 to about 48 hours, or about 24 to about 48 hours before) the inducing step. In some embodiments, the contacting step occurs simultaneously with the inducing step. In some embodiments, the antibody is an immunomodulating agent and the contacting step is conducted in the presence of immune cells. In some embodiments, the immune cell is an NK cell, an NKT cell, a T cell, a CAR-T cell, a CD14+ cell, a dendritic cell, and/or a PBMC cell. In some embodiments, the antibody is an immune checkpoint inhibitor. In some embodiments, the immune checkpoint inhibitor inhibits PD-1, PD-L1, PD-L2, Siglec, BTLA, and/or CTLA-4. In some embodiments, the antibody is selected from the group consisting of an anti-PD-1 antibody (e.g., nivolumab such as Opdivo®), an anti-PD-L1 antibody, an anti-CD47 antibody, anti-HER2 antibody (e.g., Trastuzmab such as Herceptin®), an anti-CD20 antibody, and an anti-CD3 antibody, or any combination thereof. In some embodiments, the antibody specifically recognizes both immune cells and tumor cells. In some embodiments, the antibody is an anti-HER2/anti-CD3 antibody, an anti-HER2/anti-CD47/anti-CD3 antibody, or an anti-PD-L1/anti-CD47/anti-CD3 antibody. In some embodiments, the reporter protein is secreted by the tumor cells. In some embodiments, the reporter protein is a secretable luciferase. In some embodiments, the determining step comprises detecting the reporter protein over different time points. In some embodiments, the tumor cells are present in a 3D spheroid with a second population of cells. In some embodiments, the second population of cells are fibroblast cells or stromal cells. In some embodiments, each of the tumor cells further comprises a second nucleic acid encoding a second reporter protein (e.g., luciferase or GFP). In some embodiments, the expression of the second nucleic acid is controlled by a second inducible promoter (e.g., TetOn). In some embodiments, the expression of the second nucleic acid is controlled by the same inducible promoter in a same vector. In some embodiments, each of the tumor cells further comprises a third nucleic acid encoding a CRISPR/Cas targeting an inhibitory checkpoint molecule (e.g., PD-L1). In some embodiments, the method further comprises contacting the tumor cells with a second cell-killing agent (e.g., small compound, immune effector cell, antibody such as multispecific antibody. ADC, immunomodulator such as immune checkpoint inhibitor, etc., or any combinations thereof). In some embodiments, the second cell-killing agent is an immune check point inhibitor (e.g., antibody) inhibiting PD-1, PD-L1, PD-L2, Siglec, BTLA, and/or CTLA-4, such as anti-PD-1 antibody. In some embodiments, the second cell-killing agent is an antibody specifically targeting the tumor cell and the immune cell (e.g., BiTE).

In one aspect the disclosure provides for a method of evaluating the effectiveness of a cell-killing agent on a population of tumor cells comprising a) contacting the tumor cells with a cell-killing agent, wherein the cell-killing agent comprises a cytotoxin, drug, small molecule, and/or small molecule compound, wherein each of the tumor cells comprises a first nucleic acid encoding a first reporter protein (e.g., luciferase) operably linked to a first inducible promoter (e.g., TetOn), and a second nucleic acid encoding a second reporter protein (e.g., GFP) operably linked to a second inducible promoter (e.g., TetOn)), b) inducing expression of both nucleic acids to produce both reporter proteins, and c) determining the amount of both reporter proteins, wherein the amount of each reporter protein negatively correlates with the effectiveness of the cell killing agent, wherein the contacting step is carried out at a cell-killing phase, and wherein the determining step is carried out at a subsequent evaluating phase. In one aspect the disclosure provides for a method of evaluating the effectiveness of a cell-killing agent on a population of tumor cells comprising a) contacting the tumor cells with a cell-killing agent, where the tumor cells are present in a 3D spheroid, wherein the cell-killing agent comprises a cytotoxin, drug, small molecule, and/or small molecule compound, wherein each of the tumor cells comprises a first nucleic acid encoding a first reporter protein (e.g., luciferase) operably linked to a first inducible promoter (e.g., TetOn), and a second nucleic acid encoding a second reporter protein (e.g., GFP) operably linked to a second inducible promoter (e.g., TetOn)), b) inducing expression of both nucleic acids to produce both reporter proteins, and c) determining the amount of both reporter proteins, wherein the amount of each reporter protein negatively correlates with the effectiveness of the cell killing agent, wherein the contacting step is carried out at a cell-killing phase, and wherein the determining step is carried out at a subsequent evaluating phase. In some embodiments, the first and second inducible promoters are the same or different. In some embodiments, the first and second nucleic acids are on the same vector or different vectors. In some embodiments, there is provided a method of evaluating the effectiveness of a cell-killing agent on a population of tumor cells comprising a) contacting the tumor cells with a cell-killing agent, wherein the cell-killing agent comprises a cytotoxin, drug, small molecule, and/or small molecule compound, wherein each of the tumor cells comprises from upstream to downstream: an inducible promoter (e.g., TetOn)—a first nucleic acid encoding a first reporter protein (e.g., luciferase)—a linking sequence (e.g., IRES or nucleic acid encoding a self-cleaving 2A peptide such as P2A)—a second nucleic acid encoding a second reporter protein (e.g., GFP), b) inducing expression of both nucleic acids to produce both reporter proteins, and c) determining the amount of both reporter proteins, wherein the amount of each reporter protein negatively correlates with the effectiveness of the cell killing agent, wherein the contacting step is carried out at a cell-killing phase, and wherein the determining step is carried out at a subsequent evaluating phase. In some embodiments, there is provided a method of evaluating the effectiveness of a cell-killing agent on a population of tumor cells comprising a) contacting the tumor cells with a cell-killing agent, where the tumor cells are present in a 3D spheroid, wherein the cell-killing agent comprises a cytotoxin, drug, small molecule, and/or small molecule compound, wherein each of the tumor cells comprises from upstream to downstream: an inducible promoter (e.g., TetOn)—a first nucleic acid encoding a first reporter protein (e.g., luciferase)—a linking sequence (e.g., IRES or nucleic acid encoding a self-cleaving 2A peptide such as P2A)—a second nucleic acid encoding a second reporter protein (e.g., GFP), b) inducing expression of both nucleic acids to produce both reporter proteins, and c) determining the amount of both reporter proteins, wherein the amount of each reporter protein negatively correlates with the effectiveness of the cell killing agent, wherein the contacting step is carried out at a cell-killing phase, and wherein the determining step is carried out at a subsequent evaluating phase. In some embodiments, the contacting step occurs before (e.g., about 4 to about 48 hours, or about 24 to about 48 hours before) the inducing step. In some embodiments, the contacting step occurs simultaneously with the inducing step. In some embodiments, the first and/or second reporter protein is secreted by the tumor cells. In some embodiments, the first and/or second reporter protein is a secretable luciferase. In some embodiments, the first reporter protein is a secretable luciferase and the second reporter protein is intracellular GFP. In some embodiments, the determining step comprises detecting the reporter protein over different time points. In some embodiments, the tumor cells are present in a 3D spheroid with a second population of cells. In some embodiments, the second population of cells are fibroblast cells or stromal cells. Thus in some embodiments, there is provided a method of evaluating the effectiveness of a cell-killing agent on a population of tumor cells comprising a) contacting the tumor cells with a cell-killing agent, where the tumor cells are present in a 3D spheroid with a second population of cells comprising fibroblast or stromal cells, wherein the cell-killing agent comprises a cytotoxin, drug, small molecule, and/or small molecule compound, wherein each of the tumor cells comprises from upstream to downstream: an inducible promoter (e.g., TetOn)—a first nucleic acid encoding a first reporter protein (e.g., luciferase)—a linking sequence (e.g., IRES or nucleic acid encoding a self-cleaving 2A peptide such as P2A)—a second nucleic acid encoding a second reporter protein (e.g., GFP), b) inducing expression of both nucleic acids to produce both reporter proteins, and c) determining the amount of both reporter proteins, wherein the amount of each reporter protein negatively correlates with the effectiveness of the cell killing agent, wherein the contacting step is carried out at a cell-killing phase, and wherein the determining step is carried out at a subsequent evaluating phase. In some embodiments, each of the tumor cells further comprises a third nucleic acid encoding a CRISPR/Cas targeting an inhibitory checkpoint molecule (e.g., PD-L1). In some embodiments, the method further comprises contacting the tumor cells with a second cell-killing agent (e.g., small compound, immune effector cell, antibody such as multispecific antibody, ADC, immunomodulator such as immune checkpoint inhibitor, etc., or any combinations thereof). In some embodiments, the second cell-killing agent is an immune check point inhibitor (e.g., antibody) inhibiting PD-1, PD-L1, PD-L2, Siglec, BTLA, and/or CTLA4, such as anti-PD-1 antibody. In some embodiments, the second cell-killing agent is an immune cell such as an NK cell, an NKT cell, a T cell, a CAR-T cell, a CD14+ cell, a dendritic cell, and/or a PBMC cell. In some embodiments, the second cell-killing agent is antibody and the contacting step is conducted in the presence of immune cells. In some embodiments, the antibody is selected from the group consisting of an anti-PD-1 antibody (e.g., nivolumab such as Opdivo®), an anti-PD-L1 antibody, an anti-CD47 antibody, an anti-HER2 antibody (e.g., Trastuzmab such as Herceptin®), an anti-CD20 antibody, and an anti-CD3 antibody, or any combination thereof. In some embodiments, the antibody specifically recognizes both immune cells and tumor cells. In some embodiments, the antibody is an anti-HER2/anti-CD3 antibody, an anti-HER2/anti-CD47/anti-CD3 antibody, or an anti-PD-L1/anti-CD47/anti-CD3 antibody.

In one aspect the disclosure provides for a method of evaluating the effectiveness of a cell-killing agent on a population of tumor cells comprising a) contacting the tumor cells with a cell-killing agent, wherein the cell-killing agent comprises an immune cell (e.g., NK, CTL, or PBMC), wherein each of the tumor cells comprises a first nucleic acid encoding a first reporter protein (e.g., luciferase) operably linked to a first inducible promoter (e.g., TetOn), and a second nucleic acid encoding a second reporter protein (e.g., GFP) operably linked to a second inducible promoter (e.g., TetOn)), b) inducing expression of both nucleic acids to produce both reporter proteins, and c) determining the amount of both reporter proteins, wherein the amount of each reporter protein negatively correlates with the effectiveness of the cell killing agent, wherein the contacting step is carried out at a cell-killing phase, and wherein the determining step is carried out at a subsequent evaluating phase. In one aspect the disclosure provides for a method of evaluating the effectiveness of a cell-killing agent on a population of tumor cells comprising a) contacting the tumor cells with a cell-killing agent, where the tumor cells are present in a 3D spheroid, wherein the cell-killing agent comprises an immune cell (e.g., NK, CTL, or PBMC), wherein each of the tumor cells comprises a first nucleic acid encoding a first reporter protein (e.g., luciferase) operably linked to a first inducible promoter (e.g., TetOn), and a second nucleic acid encoding a second reporter protein (e.g., GFP) operably linked to a second inducible promoter (e.g., TetOn)), b) inducing expression of both nucleic acids to produce both reporter proteins, and c) determining the amount of both reporter proteins, wherein the amount of each reporter protein negatively correlates with the effectiveness of the cell killing agent, wherein the contacting step is carried out at a cell-killing phase, and wherein the determining step is carried out at a subsequent evaluating phase. In some embodiments, the first and second inducible promoters are the same or different. In some embodiments, the first and second nucleic acids are on the same vector or different vectors. In some embodiments, there is provided a method of evaluating the effectiveness of a cell-killing agent on a population of tumor cells comprising a) contacting the tumor cells with a cell-killing agent, wherein the cell-killing agent comprises an immune cell (e.g., NK, CTL, or PBMC), wherein each of the tumor cells comprises from upstream to downstream: an inducible promoter (e.g., TetOn)—a first nucleic acid encoding a first reporter protein (e.g., luciferase)—a linking sequence (e.g., IRES or nucleic acid encoding a self-cleaving 2A peptide such as P2A)—a second nucleic acid encoding a second reporter protein (e.g., GFP), b) inducing expression of both nucleic acids to produce both reporter proteins, and c) determining the amount of both reporter proteins, wherein the amount of each reporter protein negatively correlates with the effectiveness of the cell killing agent, wherein the contacting step is carried out at a cell-killing phase, and wherein the determining step is carried out at a subsequent evaluating phase. In some embodiments, there is provided a method of evaluating the effectiveness of a cell-killing agent on a population of tumor cells comprising a) contacting the tumor cells with a cell-killing agent, where the tumor cells are present in a 3D spheroid, wherein the cell-killing agent comprises an immune cell (e.g., NK, CTL, or PBMC), wherein each of the tumor cells comprises from upstream to downstream: an inducible promoter (e.g., TetOn)—a first nucleic acid encoding a first reporter protein (e.g., luciferase)—a linking sequence (e.g., IRES or nucleic acid encoding a self-cleaving 2A peptide such as P2A)—a second nucleic acid encoding a second reporter protein (e.g., GFP), b) inducing expression of both nucleic acids to produce both reporter proteins, and c) determining the amount of both reporter proteins, wherein the amount of each reporter protein negatively correlates with the effectiveness of the cell killing agent, wherein the contacting step is carried out at a cell-killing phase, and wherein the determining step is carried out at a subsequent evaluating phase. In some embodiments, the contacting step occurs before (e.g., about 4 to about 48 hours, or about 24 to about 48 hours before) the inducing step. In some embodiments, the contacting step occurs simultaneously with the inducing step. In some embodiments, the immune cell is an NK cell, an NKT cell, a T cell, a CAR-T cell, a CD14+ cell, a dendritic cell, and/or a PBMC cell. In some embodiments, the first and/or second reporter protein is secreted by the tumor cells. In some embodiments, the first and/or second reporter protein is a secretable luciferase. In some embodiments, the first reporter protein is a secretable luciferase and the second reporter protein is intracellular GFP. In some embodiments, the determining step comprises detecting the reporter protein over different time points. In some embodiments, the tumor cells are present in a 3D spheroid with a second population of cells. In some embodiments, the second population of cells are fibroblast cells or stromal cells. Thus in some embodiments, there is provided a method of evaluating the effectiveness of a cell-killing agent on a population of tumor cells comprising a) contacting the tumor cells with a cell-killing agent, where the tumor cells are present in a 3D spheroid with a second population of cells comprising fibroblast or stromal cells, wherein the cell-killing agent comprises an immune cell (e.g., NK, CTL, or PBMC), wherein each of the tumor cells comprises from upstream to downstream: an inducible promoter (e.g., TetOn)—a first nucleic acid encoding a first reporter protein (e.g., luciferase)—a linking sequence (e.g., IRES or nucleic acid encoding a self-cleaving 2A peptide such as P2A)—a second nucleic acid encoding a second reporter protein (e.g., GFP), b) inducing expression of both nucleic acids to produce both reporter proteins, and c) determining the amount of both reporter proteins, wherein the amount of each reporter protein negatively correlates with the effectiveness of the cell killing agent, wherein the contacting step is carried out at a cell-killing phase, and wherein the determining step is carried out at a subsequent evaluating phase. In some embodiments, each of the tumor cells further comprises a third nucleic acid encoding a CRISPR/Cas targeting an inhibitory checkpoint molecule (e.g., PD-L1). In some embodiments, the method further comprises contacting the tumor cells with a second cell-killing agent (e.g., small compound, immune effector cell, antibody such as multispecific antibody, ADC, immunomodulator such as immune checkpoint inhibitor, etc., or any combinations thereof). In some embodiments, the second cell-killing agent is an immune check point inhibitor (e.g., antibody) inhibiting PD-1, PD-L1, PD-L2, Siglec, BTLA, and/or CTLA-4, such as anti-PD-1 antibody. In some embodiments, the second cell-killing agent is an immunomodulating antibody and the contacting step is conducted in the presence of the immune cells. In some embodiments, the antibody is selected from the group consisting of an anti-PD-1 antibody (e.g., nivolumab such as Opdivo®), an anti-PD-L1 antibody, an anti-CD47 antibody, an anti-HER2 antibody (e.g., Trastuzmab such as Herceptin®), an anti-CD20 antibody, and an anti-CD3 antibody, or any combination thereof. In some embodiments, the second cell-killing agent is an antibody specifically targeting the tumor cell and the immune cell (e.g., BiTE). In some embodiments, the antibody is an anti-HER2/anti-CD3 antibody, an anti-HER2/anti-CD47/anti-CD3 antibody, or an anti-PD-L1/anti-CD47/anti-CD3 antibody.

In one aspect the disclosure provides for a method of evaluating the effectiveness of a cell-killing agent on a population of tumor cells comprising a) contacting the tumor cells with a cell-killing agent, wherein the cell-killing agent comprises an antibody, wherein each of the tumor cells comprises a first nucleic acid encoding a first reporter protein (e.g., luciferase) operably linked to a first inducible promoter (e.g., TetOn), and a second nucleic acid encoding a second reporter protein (e.g., GFP) operably linked to a second inducible promoter (e.g., TetOn)), b) inducing expression of both nucleic acids to produce both reporter proteins, and c) determining the amount of both reporter proteins, wherein the amount of each reporter protein negatively correlates with the effectiveness of the cell killing agent, wherein the contacting step is carried out at a cell-killing phase, and wherein the determining step is carried out at a subsequent evaluating phase. In one aspect the disclosure provides for a method of evaluating the effectiveness of a cell-killing agent on a population of tumor cells comprising a) contacting the tumor cells with a cell-killing agent, where the tumor cells are present in a 3D spheroid, wherein the cell-killing agent comprises an antibody, wherein each of the tumor cells comprises a first nucleic acid encoding a first reporter protein (e.g., luciferase) operably linked to a first inducible promoter (e.g., TetOn), and a second nucleic acid encoding a second reporter protein (e.g., GFP) operably linked to a second inducible promoter (e.g., TetOn)), b) inducing expression of both nucleic acids to produce both reporter proteins, and c) determining the amount of both reporter proteins, wherein the amount of each reporter protein negatively correlates with the effectiveness of the cell killing agent, wherein the contacting step is carried out at a cell-killing phase, and wherein the determining step is carried out at a subsequent evaluating phase. In some embodiments, the first and second inducible promoters are the same or different. In some embodiments, the first and second nucleic acids are on the same vector or different vectors. In some embodiments, there is provided a method of evaluating the effectiveness of a cell-killing agent on a population of tumor cells comprising a) contacting the tumor cells with a cell-killing agent, wherein the cell-killing agent comprises an antibody, wherein each of the tumor cells comprises from upstream to downstream: an inducible promoter (e.g., TetOn)—a first nucleic acid encoding a first reporter protein (e.g., luciferase)—a linking sequence (e.g., IRES or nucleic acid encoding a self-cleaving 2A peptide such as P2A)—a second nucleic acid encoding a second reporter protein (e.g., GFP), b) inducing expression of both nucleic acids to produce both reporter proteins, and c) determining the amount of both reporter proteins, wherein the amount of each reporter protein negatively correlates with the effectiveness of the cell killing agent, wherein the contacting step is carried out at a cell-killing phase, and wherein the determining step is carried out at a subsequent evaluating phase. In some embodiments, there is provided a method of evaluating the effectiveness of a cell-killing agent on a population of tumor cells comprising a) contacting the tumor cells with a cell-killing agent, where the tumor cells are present in a 3D spheroid, wherein the cell-killing agent comprises an antibody, wherein each of the tumor cells comprises from upstream to downstream: an inducible promoter (e.g., TetOn)—a first nucleic acid encoding a first reporter protein (e.g., luciferase)—a linking sequence (e.g., IRES or nucleic acid encoding a self-cleaving 2A peptide such as P2A)—a second nucleic acid encoding a second reporter protein (e.g., GFP), b) inducing expression of both nucleic acids to produce both reporter proteins, and c) determining the amount of both reporter proteins, wherein the amount of each reporter protein negatively correlates with the effectiveness of the cell killing agent, wherein the contacting step is carried out at a cell-killing phase, and wherein the determining step is carried out at a subsequent evaluating phase. In some embodiments, the contacting step occurs before (e.g., about 4 to about 48 hours, or about 24 to about 48 hours before) the inducing step. In some embodiments, the contacting step occurs simultaneously with the inducing step. In some embodiments, the contacting step occurs with the presence of immune cells. In some embodiments, the immune cell is an NK cell, an NKT cell, a T cell, a CAR-T cell, a CD14+ cell, a dendritic cell, and/or a PBMC cell. In some embodiments, the antibody is an immune checkpoint inhibitor. In some embodiments, the immune checkpoint inhibitor inhibits PD-1, PD-L1, PD-L2, Siglec, BTLA, and/or CTLA-4. In some embodiments, the antibody is selected from the group consisting of an anti-PD-1 antibody (e.g., nivolumab such as Opdivo®), an anti-PD-L1 antibody, an anti-CD47 antibody, anti-HER2 antibody (e.g., Trastuzmab such as Herceptin®), an anti-CD20 antibody, and an anti-CD3 antibody, or any combination thereof. In some embodiments, the antibody specifically recognizes both immune cells and tumor cells. In some embodiments, the antibody is an anti-HER2/anti-CD3 antibody, an anti-HER2/anti-CD47/anti-CD3 antibody, or an anti-PD-L1/anti-CD47/anti-CD3 antibody. In some embodiments, the first and/or second reporter protein is secreted by the tumor cells. In some embodiments, the first and/or second reporter protein is a secretable luciferase. In some embodiments, the first reporter protein is a secretable luciferase and the second reporter protein is intracellular GFP. In some embodiments, the determining step comprises detecting the reporter protein over different time points. In some embodiments, the tumor cells are present in a 3D spheroid with a second population of cells. In some embodiments, the second population of cells are fibroblast cells or stromal cells. Thus in some embodiments, there is provided a method of evaluating the effectiveness of a cell-killing agent on a population of tumor cells comprising a) contacting the tumor cells with a cell-killing agent, where the tumor cells are present in a 3D spheroid with a second population of cells comprising fibroblast or stromal cells, wherein the cell-killing agent comprises an antibody, wherein each of the tumor cells comprises from upstream to downstream: an inducible promoter (e.g., TetOn)—a first nucleic acid encoding a first reporter protein (e.g., luciferase)—a linking sequence (e.g., IRES or nucleic acid encoding a self-cleaving 2A peptide such as P2A)—a second nucleic acid encoding a second reporter protein (e.g., GFP), b) inducing expression of both nucleic acids to produce both reporter proteins, and c) determining the amount of both reporter proteins, wherein the amount of each reporter protein negatively correlates with the effectiveness of the cell killing agent, wherein the contacting step is carried out at a cell-killing phase, and wherein the determining step is carried out at a subsequent evaluating phase. In some embodiments, each of the tumor cells further comprises a third nucleic acid encoding a CRISPR/Cas targeting an inhibitory checkpoint molecule (e.g., PD-L1). In some embodiments, the method further comprises contacting the tumor cells with a third cell-killing agent (e.g., small compound, immune effector cell, antibody such as multispecific antibody, ADC, immunomodulator such as immune checkpoint inhibitor, etc., or any combinations thereof). In some embodiments, the third cell-killing agent is an immune check point inhibitor (e.g., antibody) inhibiting PD-1, PD-L 1, PD-L2, Siglec, BTLA, and/or CTLA-4, such as anti-PD-1 antibody. In some embodiments, the third cell-killing agent is an antibody specifically targeting the tumor cell and the immune cell (e.g., BiTE).

In one aspect the disclosure provides for a method of evaluating the effectiveness of a cell-killing agent on a population of tumor cells comprising a) contacting the tumor cells with a cell-killing agent, wherein the cell-killing agent comprises an immune cell and an antibody, wherein each of the tumor cells comprises a first nucleic acid encoding a first reporter protein (e.g., luciferase) operably linked to a first inducible promoter (e.g., TetOn), and a second nucleic acid encoding a second reporter protein (e.g., GFP) operably linked to a second inducible promoter (e.g., TetOn)), b) inducing expression of both nucleic acids to produce both reporter proteins, and c) determining the amount of both reporter proteins, wherein the amount of each reporter protein negatively correlates with the effectiveness of the cell killing agent, wherein the contacting step is carried out at a cell-killing phase, and wherein the determining step is carried out at a subsequent evaluating phase. In one aspect the disclosure provides for a method of evaluating the effectiveness of a cell-killing agent on a population of tumor cells comprising a) contacting the tumor cells with a cell-killing agent, where the tumor cells are present in a 3D spheroid, wherein the cell-killing agent comprises an immune cell and an antibody, wherein each of the tumor cells comprises a first nucleic acid encoding a first reporter protein (e.g., luciferase) operably linked to a first inducible promoter (e.g., TetOn), and a second nucleic acid encoding a second reporter protein (e.g., GFP) operably linked to a second inducible promoter (e.g., TetOn)), b) inducing expression of both nucleic acids to produce both reporter proteins, and c) determining the amount of both reporter proteins, wherein the amount of each reporter protein negatively correlates with the effectiveness of the cell killing agent, wherein the contacting step is carried out at a cell-killing phase, and wherein the determining step is carried out at a subsequent evaluating phase. In some embodiments, the first and second inducible promoters are the same or different. In some embodiments, the first and second nucleic acids are on the same vector or different vectors. In some embodiments, there is provided a method of evaluating the effectiveness of a cell-killing agent on a population of tumor cells comprising a) contacting the tumor cells with a cell-killing agent, wherein the cell-killing agent comprises an immune cell and an antibody, wherein each of the tumor cells comprises from upstream to downstream: an inducible promoter (e.g., TetOn)—a first nucleic acid encoding a first reporter protein (e.g., luciferase)—a linking sequence (e.g., IRES or nucleic acid encoding a self-cleaving 2A peptide such as P2A)—a second nucleic acid encoding a second reporter protein (e.g., GFP), b) inducing expression of both nucleic acids to produce both reporter proteins, and c) determining the amount of both reporter proteins, wherein the amount of each reporter protein negatively correlates with the effectiveness of the cell killing agent, wherein the contacting step is carried out at a cell-killing phase, and wherein the determining step is carried out at a subsequent evaluating phase. In some embodiments, there is provided a method of evaluating the effectiveness of a cell-killing agent on a population of tumor cells comprising a) contacting the tumor cells with a cell-killing agent, where the tumor cells are present in a 3D spheroid, wherein the cell-killing agent comprises an immune cell and an antibody, wherein each of the tumor cells comprises from upstream to downstream: an inducible promoter (e.g., TetOn)—a first nucleic acid encoding a first reporter protein (e.g., luciferase)—a linking sequence (e.g., IRES or nucleic acid encoding a self-cleaving 2A peptide such as P2A)—a second nucleic acid encoding a second reporter protein (e.g., GFP), b) inducing expression of both nucleic acids to produce both reporter proteins, and c) determining the amount of both reporter proteins, wherein the amount of each reporter protein negatively correlates with the effectiveness of the cell killing agent, wherein the contacting step is carried out at a cell-killing phase, and wherein the determining step is carried out at a subsequent evaluating phase. In some embodiments, the contacting step occurs before (e.g., about 4 to about 48 hours, or about 24 to about 48 hours before) the inducing step. In some embodiments, the contacting step occurs simultaneously with the inducing step. In some embodiments, the immune cell is an NK cell, an NKT cell, a T cell, a CAR-T cell, a CD14+ cell, a dendritic cell, and/or a PBMC cell. In some embodiments, the antibody is an immunomodulating agent and the contacting step is conducted in the presence of immune cells. In some embodiments, the antibody is an immune checkpoint inhibitor. In some embodiments, the immune checkpoint inhibitor inhibits PD-1, PD-L1, PD-L2, Siglec, BTLA, and/or CTLA-4. In some embodiments, the antibody is selected from the group consisting of an anti-PD-1 antibody (e.g., nivolumab such as Opdivo®), an anti-PD-L1 antibody, an anti-CD47 antibody, an anti-HER2 antibody (e.g., Trastuzmab such as Herceptin®), an anti-CD20 antibody, and an anti-CD3 antibody, or any combination thereof. In some embodiments, the antibody specifically recognizes both immune cells and tumor cells. In some embodiments, the antibody is an anti-HER2/anti-CD3 antibody, an anti-HER2/anti-CD47/anti-CD3 antibody, or an anti-PD-L1/anti-CD47/anti-CD3 antibody. In some embodiments, the first and/or second reporter protein is secreted by the tumor cells. In some embodiments, the first and/or second reporter protein is a secretable luciferase. In some embodiments, the first reporter protein is a secretable luciferase and the second reporter protein is intracellular GFP. In some embodiments, the determining step comprises detecting the reporter protein over different time points. In some embodiments, the tumor cells are present in a 3D spheroid with a second population of cells. In some embodiments, the second population of cells are fibroblast cells or stromal cells. Thus in some embodiments, there is provided a method of evaluating the effectiveness of a cell-killing agent on a population of tumor cells comprising a) contacting the tumor cells with a cell-killing agent, where the tumor cells are present in a 3D spheroid with a second population of cells comprising fibroblast or stromal cells, wherein the cell-killing agent comprises an immune cell and an antibody, wherein each of the tumor cells comprises from upstream to downstream: an inducible promoter (e.g., TetOn)—a first nucleic acid encoding a first reporter protein (e.g., luciferase)—a linking sequence (e.g., IRES or nucleic acid encoding a self-cleaving 2A peptide such as P2A)—a second nucleic acid encoding a second reporter protein (e.g., GFP), b) inducing expression of both nucleic acids to produce both reporter proteins, and c) determining the amount of both reporter proteins, wherein the amount of each reporter protein negatively correlates with the effectiveness of the cell killing agent, wherein the contacting step is carried out at a cell-killing phase, and wherein the determining step is carried out at a subsequent evaluating phase. In some embodiments, each of the tumor cells further comprises a third nucleic acid encoding a CRISPR/Cas targeting an inhibitory checkpoint molecule (e.g., PD-L1). In some embodiments, the method further comprises contacting the tumor cells with a second cell-killing agent (e.g., small compound, immune effector cell, antibody such as multispecific antibody, ADC, immunomodulator such as immune checkpoint inhibitor, etc., or any combinations thereof). In some embodiments, the second cell-killing agent is an immune check point inhibitor (e.g., antibody) inhibiting PD-1, PD-L1, PD-L2, Siglec, BTLA, and/or CTLA4, such as anti-PD-1 antibody. In some embodiments, the second cell-killing agent is an antibody specifically targeting the tumor cell and the immune cell (e.g., BiTE).

In one aspect the disclosure provides for a method of evaluating the effectiveness of a cell-killing agent (e.g., small compound, immune effector cell, antibody such as multispecific antibody, ADC, immunomodulator such as immune checkpoint inhibitor, etc., or any combinations thereof) on a population of tumor cells comprising a) contacting the tumor cells with a cell-killing agent, where the tumor cells are present in a 3D spheroid, wherein the cell-killing agent comprises an antibody, wherein the contacting is carried out in the presence of immune cells, wherein each of the tumor cells comprises a nucleic acid encoding a reporter protein (e.g., luciferase or GFP), wherein expression of the nucleic acid is controlled by an inducible promoter (e.g., TetOn), b) inducing expression of the nucleic acid to produce the reporter protein, and c) determining the amount of the reporter protein, wherein the amount of the reporter protein negatively correlates with the effectiveness of the cell killing agent, wherein the contacting step is carried out at a cell-killing phase, and wherein the determining step is carried out at a subsequent evaluating phase. In some embodiments, the contacting step occurs before (e.g., about 4 to about 48 hours, or about 24 to about 48 hours before) the inducing step. In some embodiments, the contacting step occurs simultaneously with the inducing step. In some embodiments, the immune cell is an NK cell, an NKT cell, a T cell, a CAR-T cell, a CD14+ cell, a dendritic cell, and/or a PBMC cell. In some embodiments, the antibody is an immunomodulating agent and the contacting step is conducted in the presence of immune cells. In some embodiments, the antibody is an immune checkpoint inhibitor. In some embodiments, the immune checkpoint inhibitor inhibits PD-1, PD-L1, PD-L2, Siglec, BTLA, and/or CTLA-4. In some embodiments, the antibody is selected from the group consisting of an anti-PD-1 antibody (e.g., nivolumab such as Opdivo®), an anti-PD-L1 antibody, an anti-CD47 antibody, an anti-HER2 antibody (e.g., Trastuzmab such as Herceptin®), an anti-CD20 antibody, and an anti-CD3 antibody, or any combination thereof. In some embodiments, the antibody specifically recognizes both immune cells and tumor cells. In some embodiments, the antibody is an anti-HER2/anti-CD3 antibody, an anti-HER2/anti-CD47/anti-CD3 antibody, or an anti-PD-L1/anti-CD47/anti-CD3 antibody. In some embodiments, the reporter protein is secreted by the tumor cells. In some embodiments, the reporter protein is a secretable luciferase. In some embodiments, the determining step comprises detecting the reporter protein over different time points. In some embodiments, the tumor cells are present in a 3D spheroid with a second population of cells. In some embodiments, the second population of cells are fibroblast cells or stromal cells. In some embodiments, each of the tumor cells further comprises a second nucleic acid encoding a second reporter protein (e.g., luciferase or GFP). In some embodiments, the expression of the second nucleic acid is controlled by a second inducible promoter (e.g., TetOn). In some embodiments, the expression of the second nucleic acid is controlled by the same inducible promoter in a same vector. In some embodiments, each of the tumor cells further comprises a third nucleic acid encoding a CRISPR/Cas targeting an inhibitory checkpoint molecule (e.g., PD-L1). In some embodiments, the method further comprises contacting the tumor cells with a third cell-killing agent (e.g., small compound, immune effector cell, antibody such as multispecific antibody, ADC, immunomodulator such as immune checkpoint inhibitor, etc., or any combinations thereof). In some embodiments, the third cell-killing agent is an immune check point inhibitor (e.g., antibody) inhibiting PD-1, PD-LL. PD-L2, Siglec, BTLA, and/or CTLA-4, such as anti-PD-1 antibody. In some embodiments, the third cell-killing agent is an antibody specifically targeting the tumor cell and the immune cell (e.g., BiTE).

In one aspect the disclosure provides for a method of evaluating the effectiveness of a cell-killing agent (e.g., small compound, immune effector cell, antibody such as multispecific antibody, ADC, immunomodulator such as immune checkpoint inhibitor, etc., or any combinations thereof) on a population of tumor cells comprising a) contacting the tumor cells with a cell-killing agent, wherein each of the tumor cells comprises a nucleic acid encoding a reporter protein (e.g., luciferase or GFP), wherein the reporter protein is secreted by tumor cells, and wherein expression of the nucleic acid is controlled by an inducible promoter (e.g., TetOn), b) inducing expression of the nucleic acid to produce the reporter protein, and c) determining the amount of the reporter protein, wherein the amount of the reporter protein negatively correlates with the effectiveness of the cell killing agent, wherein the contacting step is carried out at a cell-killing phase, and wherein the determining step is carried out at a subsequent evaluating phase. In some embodiments, the contacting step occurs before (e.g., about 4 to about 48 hours, or about 24 to about 48 hours before) the inducing step. In some embodiments, the contacting step occurs simultaneously with the inducing step. In some embodiments, the determining step comprises detecting the reporter protein over different time points. In some embodiments, the tumor cells are present in a 3D spheroid with a second population of cells. In some embodiments, the second population of cells are fibroblast cells or stromal cells. In some embodiments, the reporter protein is a secretable luciferase. In some embodiments, the reporter protein is selected from the group consisting of: Oplophorus luciferase, beetle luciferase, Renilla luciferase, Metridia luciferase, Gaussia luciferase, secreted alkaline phosphatase, secreted fluorescent protein, and NANOLUC luciferase, or any combination thereof. In some embodiments, each of the tumor cells further comprises a second nucleic acid encoding a second reporter protein (e.g., luciferase or GFP). In some embodiments, the expression of the second nucleic acid is controlled by a second inducible promoter (e.g., TetOn). In some embodiments, the expression of the second nucleic acid is controlled by the same inducible promoter in a same vector. In some embodiments, each of the tumor cells further comprises a third nucleic acid encoding a CRISPR/Cas targeting an inhibitory checkpoint molecule (e.g., PD-L1).

In one aspect the disclosure provides for a method of evaluating the effectiveness of a cell-killing agent (e.g., small compound, immune effector cell, antibody such as multispecific antibody, ADC, immunomodulator such as immune checkpoint inhibitor, etc., or any combinations thereof) on a population of tumor cells comprising a) contacting the tumor cells with a cell-killing agent, where the tumor cells are present in a 3D spheroid, wherein each of the tumor cells comprises a nucleic acid encoding a reporter protein, wherein the reporter protein is a secretable protein (such as luciferase) that is secreted by tumor cells, and wherein expression of the nucleic acid is controlled by an inducible promoter (e.g., TetOn), b) inducing expression of the nucleic acid to produce the reporter protein, and c) determining the amount of the reporter protein, wherein the amount of the reporter protein negatively correlates with the effectiveness of the cell killing agent, wherein the contacting step is carried out at a cell-killing phase, and wherein the determining step is carried out at a subsequent evaluating phase. In some embodiments, the contacting step occurs before (e.g., about 4 to about 48 hours, or about 24 to about 48 hours before) the inducing step. In some embodiments, the contacting step occurs simultaneously with the inducing step. In some embodiments, the determining step comprises detecting the reporter protein over different time points. In some embodiments, the tumor cells are present in a 3D spheroid with a second population of cells. In some embodiments, the second population of cells are fibroblast cells or stromal cells. In some embodiments, the reporter protein is selected from the group consisting of: Oplophorus luciferase, beetle luciferase, Renilla luciferase, Metridia luciferase, Gaussia luciferase, secreted alkaline phosphatase, secreted fluorescent protein, and NANOLUC luciferase, or any combination thereof. In some embodiments, the cell-killing agent comprises an immune cell (e.g., NK, CTL, or PBMC). In some embodiments, the cell-killing agent comprises an antibody (e.g., against tumor antigen). In some embodiments, the antibody is an immunomodulating agent (e.g., immune checkpoint inhibitor, or antibody specifically targeting the tumor cell and an immune cell) and the contacting step is conducted in the presence of immune cells. In some embodiments, each of the tumor cells further comprises a second nucleic acid encoding a second reporter protein (e.g., luciferase or GFP). In some embodiments, the expression of the second nucleic acid is controlled by a second inducible promoter (e.g., TetOn). In some embodiments, the expression of the second nucleic acid is controlled by the same inducible promoter in a same vector. In some embodiments, each of the tumor cells further comprises a third nucleic acid encoding a CRISPR/Cas targeting an inhibitory checkpoint molecule (e.g., PD-L1).

In one aspect the disclosure provides for a method of evaluating the effectiveness of a cell-killing agent (e.g., small compound, immune effector cell, antibody such as multispecific antibody, ADC, immunomodulator such as immune checkpoint inhibitor, etc., or any combinations thereof) on a population of tumor cells comprising a) contacting the tumor cells with a cell-killing agent, where the tumor cells are present in a 3D spheroid with a second population of cells comprising fibroblast or stromal cells, wherein each of the tumor cells comprises a nucleic acid encoding a reporter protein, wherein the reporter protein is a secretable reporter protein (such as luciferase) that is secreted by tumor cells, and wherein expression of the nucleic acid is controlled by an inducible promoter (e.g., TetOn), b) inducing expression of the nucleic acid to produce the reporter protein, and c) determining the amount of the reporter protein, wherein the amount of the reporter protein negatively correlates with the effectiveness of the cell killing agent, wherein the contacting step is carried out at a cell-killing phase, and wherein the determining step is carried out at a subsequent evaluating phase. In some embodiments, the contacting step occurs before (e.g., about 4 to about 48 hours, or about 24 to about 48 hours before) the inducing step. In some embodiments, the contacting step occurs simultaneously with the inducing step. In some embodiments, the cell-killing agent comprises an immune cell (e.g., NK, CTL, or PBMC). In some embodiments, the cell-killing agent comprises an antibody (e.g., against tumor antigen). In some embodiments, the antibody is an immunomodulating agent (e.g., immune checkpoint inhibitor, or antibody specifically targeting the tumor cell and an immune cell) and the contacting step is conducted in the presence of immune cells. In some embodiments, the determining step comprises detecting the reporter protein over different time points. In some embodiments, the reporter protein is selected from the group consisting of: Oplophorus luciferase, beetle luciferase, Renilla luciferase, Metridia luciferase, Gaussia luciferase, secreted alkaline phosphatase, secreted fluorescent protein, and NANOLUC luciferase, or any combination thereof. In some embodiments, each of the tumor cells further comprises a second nucleic acid encoding a second reporter protein (e.g., luciferase or GFP). In some embodiments, the expression of the second nucleic acid is controlled by a second inducible promoter (e.g., TetOn). In some embodiments, the expression of the second nucleic acid is controlled by the same inducible promoter in a same vector. In some embodiments, each of the tumor cells further comprises a third nucleic acid encoding a CRISPR/Cas targeting an inhibitory checkpoint molecule (e.g., PD-L1).

In one aspect the disclosure provides for a method of evaluating the effectiveness of a cell-killing agent (e.g., small compound, immune effector cell, antibody such as multispecific antibody, ADC, immunomodulator such as immune checkpoint inhibitor, etc., or any combinations thereof) on a population of tumor cells comprising a) contacting the tumor cells with a cell-killing agent, where the tumor cells are present in a 3D spheroid with a second population of cells comprising fibroblast or stromal cells, wherein each of the tumor cells comprises a nucleic acid encoding a reporter protein, wherein the reporter protein is a secretable protein (such as luciferase) that is secreted by tumor cells, wherein the tumor cells further comprise a second nucleic acid encoding a second reporter protein, wherein the second reporter protein is an intracellular reporter protein (such as GFP), and wherein expression of both nucleic acids is controlled by an inducible promoter (e.g., TetOn), b) inducing expression of both nucleic acids to produce the reporter proteins, and c) determining each amount of the reporter proteins, wherein each amount of the reporter proteins negatively correlates with the effectiveness of the cell killing agent, wherein the contacting step is carried out at a cell-killing phase, and wherein the determining step is carried out at a subsequent evaluating phase. In some embodiments, the contacting step occurs before (e.g., about 4 to about 48 hours, or about 24 to about 48 hours before) the inducing step. In some embodiments, the contacting step occurs simultaneously with the inducing step. In some embodiments, the cell-killing agent comprises an immune cell (e.g., NK, CTL, PBMC). In some embodiments, the cell-killing agent comprises an antibody (e.g., against tumor antigen). In some embodiments, the antibody is an immunomodulating agent (e.g., immune checkpoint inhibitor, or antibody specifically targeting the tumor cell and an immune cell) and the contacting step is conducted in the presence of immune cells. In some embodiments, the determining step comprises detecting each reporter protein over different time points. In some embodiments, the first and/or second reporter protein is selected from the group consisting of: Oplophorus luciferase, beetle luciferase, Renilla luciferase, Metridia luciferase, Gaussia luciferase, secreted alkaline phosphatase, secreted fluorescent protein, and NANOLUC luciferase, or any combination thereof. In some embodiments, the second reporter protein is GFP. In some embodiments, the nucleic acids encoding the first and second reporter proteins are on the same vector both under the same inducible promoter control. In some embodiments, the nucleic acids encoding the first and second reporter proteins are connected via IRES or a self-cleaving 2A peptide, such as P2A, T2A, E2A, F2A, BmCPV 2A, BmIFV 2A. In some embodiments, each of the tumor cells further comprises a third nucleic acid encoding a CRISPR/Cas targeting an inhibitory checkpoint molecule (e.g., PD-L1). In some embodiments, the method further comprises contacting the tumor cells with a second cell-killing agent (e.g., small compound, immune effector cell, antibody such as multispecific antibody, ADC, immunomodulator such as immune checkpoint inhibitor, etc., or any combinations thereof). In some embodiments, the second cell-killing agent is an immune check point inhibitor (e.g., antibody) inhibiting PD-1, PD-L1, PD-L2, Siglec, BTLA, and/or CTLA-4, such as anti-PD-1 Ab.

In one aspect the disclosure provides for a method of evaluating the effectiveness of a cell-killing agent on a population of tumor cells comprising a) contacting the tumor cells with a cell-killing agent, where the tumor cells are present in a 3D spheroid with a second population of cells comprising fibroblast or stromal cells, wherein the cell-killing agent comprises an immune cell (e.g., NK, CTL, PBMC) and an antibody, wherein each of the tumor cells comprises a nucleic acid encoding a reporter protein, wherein the reporter protein is a secretable protein (such as luciferase) that is secreted by tumor cells, wherein the tumor cells further comprise a second nucleic acid encoding a second reporter protein, wherein the second reporter protein is an intracellular reporter protein (such as GFP), and wherein expression of both nucleic acids is controlled by an inducible promoter, b) inducing expression of both nucleic acids to produce the reporter proteins, and c) determining each amount of the reporter proteins, wherein each amount of the reporter protein negatively correlates with the effectiveness of the cell killing agent, wherein the contacting step is carried out at a cell-killing phase, and wherein the determining step is carried out at a subsequent evaluating phase. In some embodiments, the contacting step occurs before (e.g., about 4 to about 48 hours, or about 24 to about 48 hours before) the inducing step. In some embodiments, the contacting step occurs simultaneously with the inducing step. In some embodiments, the immune cell is an NK cell, an NKT cell, a T cell, a CAR-T cell, a CD14+ cell, a dendritic cell, and/or a PBMC cell. In some embodiments, the antibody is an immunomodulating agent and the contacting step is conducted in the presence of immune cells. In some embodiments, the antibody is an immune checkpoint inhibitor. In some embodiments, the immune checkpoint inhibitor (e.g., Ab) inhibits PD-1, PD-L1, PD-L2, Siglec, BTLA, and/or CTLA-4. In some embodiments, the antibody is selected from the group consisting of an anti-PD-1 antibody (e.g., nivolumab such as Opdivo®), an anti-PD-L1 antibody, an anti-CD47 antibody, an anti-HER2 antibody (e.g., Trastuzmab such as Herceptin®), an anti-CD20 antibody, and an anti-CD3 antibody, or any combination thereof. In some embodiments, the antibody is multispecific (e.g., an anti-HER2/anti-CD3 antibody, an anti-HER2/anti-CD47/anti-CD3 antibody, or an anti-PD-L1/anti-CD47/anti-CD3 antibody). In some embodiments, the determining step comprises detecting each reporter protein over different time points. In some embodiments, the first and/or second reporter protein is selected from the group consisting of: Oplophorus luciferase, beetle luciferase, Renilla luciferase, Metridia luciferase, Gaussia luciferase, secreted alkaline phosphatase, secreted fluorescent protein, and NANOLUC luciferase, or any combination thereof. In some embodiments, the second reporter protein is GFP. In some embodiments, the nucleic acids encoding the first and second reporter proteins are on the same vector both under the same inducible promoter control. In some embodiments, the nucleic acids encoding the first and second reporter proteins are connected via IRES or a self-cleaving 2A peptide, such as P2A, T2A, E2A, F2A, BmCPV 2A, BmIFV 2A. In some embodiments, each of the tumor cells further comprises a third nucleic acid encoding a CRISPR/Cas targeting an inhibitory checkpoint molecule (e.g., PD-L1). In some embodiments, the method further comprises contacting the tumor cells with a third cell-killing agent (e.g., small compound, immune effector cell, antibody such as multispecific antibody, ADC, immunomodulator such as immune checkpoint inhibitor, etc., or any combinations thereof). In some embodiments, the third cell-killing agent is an immune check point inhibitor (e.g., antibody) inhibiting PD-1, PD-L1, PD-L2, Siglec, BTLA, and/or CTLA-4, such as anti-PD-1 Ab.

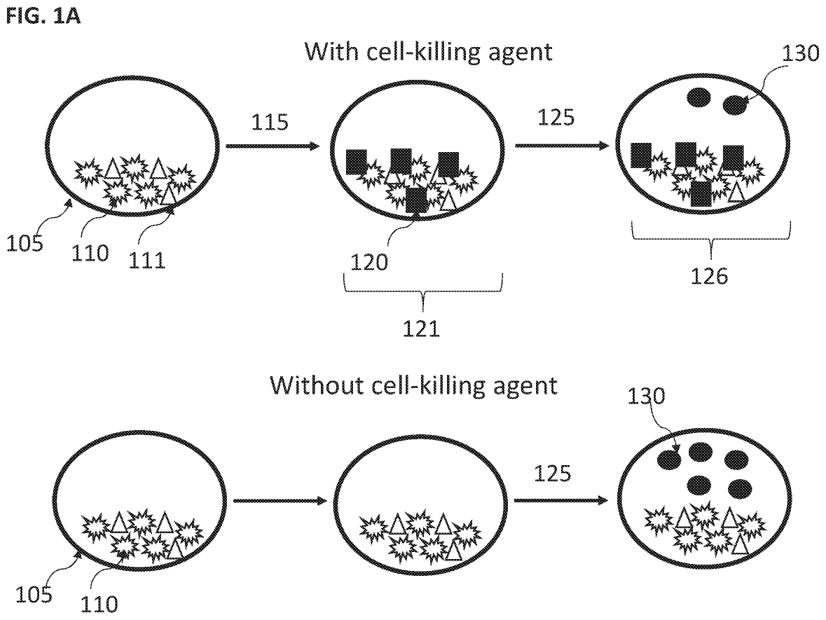

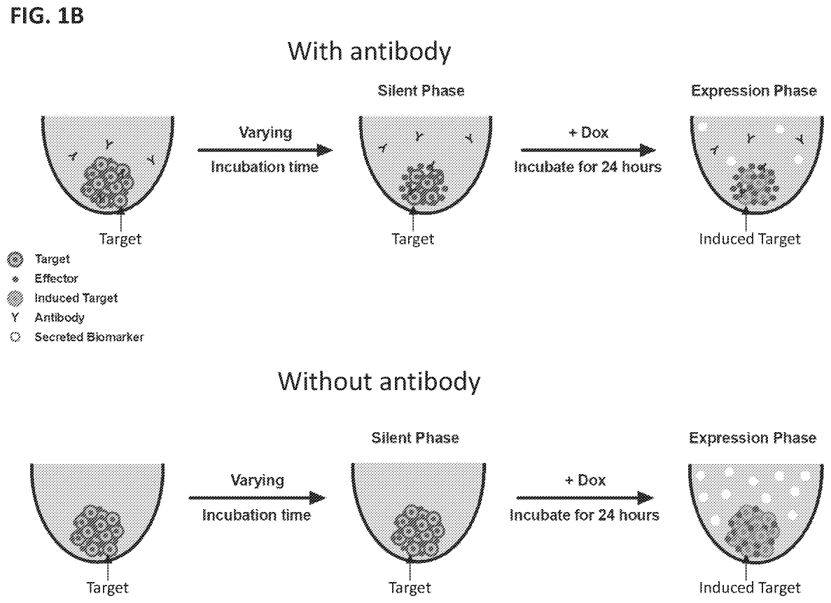

FIG. 1A depicts an exemplary embodiment of the methods of the disclosure. In some embodiments, tumor cells 110 can be cultured in media 105. In some embodiments, tumor cells 110 can be grown in a three-dimensional spheroid. In some embodiments, tumor cells 110 can be grown in a spheroid with a second population of cells 111. In some embodiments, the second population of cells 111 can be tumor microenvironment promoting cells, such as fibroblasts. In some embodiments, the tumor cells 110 can comprise a nucleic acid encoding a reporter protein, operably linked to an inducible promoter. In some embodiments, the reporter protein can be secretable. Because the reporter protein can be secretable, culture of the tumor cells with a second population of cells 111 will not dilute the reporter protein signal (i.e., detection of the reporter protein in the media). This can allow tumor cells to be grown in biologically-relevant three-dimensional conditions, with any additional and/or supporting cell type, without reducing the ability to detect cell-mediated killing of the tumor cells by a cell-killing agent.

In some embodiments, the tumor cells 110 can be contacted through step 115 with a cell-killing agent 120. The cell-killing agent 120 can kill the tumor cells 110 either directly or indirectly. Contacting the tumor cell with the cell-killing agent starts the silent phase time period 121. After a certain amount of incubation time with the cell-killing agent, or simultaneously with addition of the cell-killing agent, nucleic acids within the tumor cells encoding the secretable reporter protein can be induced as in step 125 (e.g., with an induction agent). Induction results in expression and secretion of the secretable reporter protein 130 into the media 105 in which the tumor cells 110 are growing. Induction starts the expression phase 126. In some embodiments, the control sample does not comprise the cell-killing agent. In the control sample, tumor cells 110 are cultured in media 105. After a certain amount of incubation time with the cell-killing agent, or simultaneously with addition of the cell-killing agent, nucleic acids within the tumor cells encoding the secretable reporter protein can be induced as in step 125 (e.g., with an induction agent). Induction results in expression and secretion of the secretable reporter protein 130 into the media in which the tumor cells 110 are growing. By comparing the amount of reporter protein that is produced between the sample with cell-killing agent and the control sample without cell-killing agent, the amount of cell-killing by the cell-killing agent can be determined. The more cell-killing that occurs in the method (e.g., due to the cell-killing agent), the lower the amount of secreted reporter protein is detected in the media. The less cell-killing that occurs in the method, the higher the amount of secreted reporter protein is detected in the media. In other words, the amount of reporter protein negatively correlates with the effectiveness of the cell-killing agent in killing tumor cells.

FIG. 1B depicts another exemplary embodiment of the methods of the disclosure. Tumor cells comprising a nucleic acid encoding a secretable reporter protein, operably linked to an inducible promoter, can be contacted with a cell-killing agent, such as an antibody and an effector cell (e.g., T cells such as cytotoxic T cells) that can be used in antibody-dependent cell-mediated cytotoxicity (ADCC). The antibody can be an immunomodulatory agent. Cell-killing via the cell-killing agent can occur during this time (i.e., silent phase). After a certain amount of incubation time with the cell-killing agent, or simultaneously with addition of the cell-killing agent (e.g., antibody), expression of the secretable reporter protein is induced (such as with doxycycline). Induction results in expression and secretion of the secretable reporter protein into the media in which the tumor cells are growing (i.e., expression phase). In some embodiments, the control sample does not comprise the cell-killing agent (e.g., antibody). In some embodiments, in the control sample, tumor cells are cultured in media with effector cells (e.g., T cells such as cytotoxic T cells). In some embodiments, the control sample does not comprise an effector cell. After a certain amount of incubation time with the cell-killing agent, or simultaneously with addition of the cell-killing agent, the nucleic acid within tumor cells encoding the secretable reporter protein can be induced (e.g., with doxycycline). Induction results in expression and secretion of the secretable reporter protein into the media in which the tumor cells are growing. By comparing the amount of reporter protein that is produced between the sample with the antibody and the control sample without the antibody, or between the sample with the effector cell and the control sample without the effector cell, the amount of effector cell-killing mediated by the antibody can be determined. The more cell-killing that occurs in the method (e.g., due to antibody-mediated effector cell killing), the lower the amount of secreted reporter protein can be detected in the media. The less cell-killing that occurs in the method, the higher the amount of secreted reporter protein can be detected in the media. In other words, the amount of reporter protein negatively correlates with the effectiveness of ADCC.

### Cell Culture Methods

The disclosure provides for methods for evaluating the effect of a cell-killing agent on a population of tumor cells. The tumor cells can be cultured in standard tissue culture dishes e.g. multidishes and microwell plates, or in other vessels, as desired. The methods of the disclosure can be conducted in a 96 well, 386-well or other multi-well plates, microfluidic devices, capillaries and the like.

Tumor cells used in the assay can be cultured in a two-dimensional monolayer, a three-dimensional spheroid, or in any three-dimensional structure. Tumor cells can be grown on a three-dimensional support to generate tumor spheroids. Tumor spheroids can be generated using methods such as, hanging drops, culture of cells on non-adherent surfaces, spinner flask, NASA rotary cell culture system, multilayer microfluidic devices with a porous membrane, microfluidic arrays comprising concave microwells and flat cell culture chambers, and the like.

In some embodiments, tumor spheroids are generated by culture in ultra-low-attachment plates (e.g., from Corning). In some embodiments, tumor spheroids are generated by culture of tumor cells with an equivalent number of dermal human fibroblast cells in ultra-low-attachment plates. The plates may be incubated in a shaker at 200 rpm for from 1 to 6 days. In some instances, the plates may be incubated in a shaker at 200 rpm for four days.

Tumor spheroids can be produced, by for example, by (1) organotypic explant cultures, in which whole organs or organ elements or slices are harvested and grown on a substrate in media: (2) stationary or rotating microcarrier cultures, in which dissociated cells aggregate around porous circular or cylindrical substrates with adhesive properties; (3) micromass cultures, in which cells are pelleted and suspended in media containing appropriate amounts of nutrients and differentiation factors; (4) free cells in a rotating vessel that adhere to one another and eventually form tissue or organ-like structures (so-called rotating wall vessels or microgravity bioreactors); and (5) gel-based techniques, in which cells are embedded in a substrate, such as agarose or matrigel, that may or may not contain a scaffolding of collagen or other organic or synthetic fiber which mimics the ECM. The tumor spheroids can be cultured with or without non-tumor cells for at least 1 day, at least 2 days, at least 3 days, at least 4 days, at least 6 days, at least 1 week, or at least 2 weeks.

Tumor cells comprising a nucleic acid encoding a reporter protein under control of an inducible promoter can be contacted with a cell killing agent, such as by incubation, co-culture, co-transduction (e.g., co-transduction of a KO construct for PD-L1 KO), diffusion, osmosis, and the like. This starts the silent phase. During the silent phase, cell killing can occur.

The cell-killing agents can be used to induce antibody-dependent effector cell-mediated cytotoxicity (ADCC) against a tumor cell. To this end, cell-killing agents can be administered freely in a physiologically acceptable solution, (e.g., media, cell culture solution, buffers). Where cell-killing agents act directly they may be administered directly to the tumor cells. Where the cell-killing agents act indirectly they may be mixed together first before contacting the tumor cells, or they may be added sequentially or simultaneously to the tumor cell culture. For example, an effector cell (e.g., NK cell, CTL, or PBMC) and an immunotherapy agent (or using any other methods to generate activated effectors) can be mixed first thereby forming an activated effector cell. The activated effector cell is then contacted with the tumor cell culture, and the activated effector cell can kill the tumor cell via the immunotherapy. In another example, the effector cell and the immunotherapy can separately be added to the tumor cell culture, simultaneously or sequentially.

The cell-killing agent can be added to the media in which tumor cells are growing in any concentration. For example, the cell-killing agent can be added at a concentration ranging from about 0 ng/mL to about 3000 ng/mL, such as any of about 0 ng/mL to about 2000 ng/mL, about 0 ng/mL to about 1000 ng/mL, about 0 ng/mL to about 500 ng/mL, about 0 ng/mL to about 200 ng/mL, or about 0 ng/mL to about 100 ng/mL. For example, the cell-killing agent can be added at a concentration ranging from 0.0128 ng/mL to 40 ng/mL. In some embodiments, the cell-killing agent can be added at a concentration of at least 0.001, 0.01, 0.1, 1, 10, or 100 ng/mL. In some embodiments, the cell-killing agent can be added at a concentration of at most 0.001, 0.01, 0.1, 1, 10, or 100 ng/mL. In some embodiments, the cell-killing agent can be added with a serial dilution, such as 2-fold or 5-fold serial dilution.

Incubation of the tumor cells and the cell-killing agent in the silent phase can occur for at least about any of 30 min, 1, 2, 3, 4, 5, 6, 12, 24, 36, 48, 54, 60, 66, or 72 hours or more. Incubation of the tumor cells and the cell-killing agent in the silent phase can occur for at most about any of 30 min, 1, 2, 3, 4, 5, 6, 12, 24, 36, 48, 54, 60, 66, or 72 hours. Incubation of the tumor cells and the cell-killing agent in the silent phase can occur for at least about any of 1, 2, 3, 4, 5, 6, 7, 8, 9, or 10, or more days. Incubation of the tumor cells and the cell-killing agent in the silent phase can occur for at most about any of 1, 2, 3, 4, 5, 6, 7, 8, 9, or 10 days. In some embodiments, the tumor cells and the cell-killing agent are incubated during the silent phase for at least about 24 hours. In some embodiments, the tumor cells and the cell-killing agent are incubated during the silent phase for about 4 to about 48 hours, such as any of about 4 to about 8 hours, about 12 to about 48 hours, about 24 to about 48 hours, about 4 to about 24 hours, or about 12 to about 24 hours. In some embodiments, the tumor cells and the cell-killing agent are incubated during the silent phase for up to about 6 days (e.g., about any of 1, 2, 3, 4, 5, or 6 days). In some instances, the tumor cells and the cell-killing agent are incubated during the silent phase for about 24 hours. In some instances, the tumor cells and the cell-killing agent are incubated during the silent phase for about 48 hours.

### Methods of Induction

The silent phase can end when the sample is induced to begin expression of the reporter protein. In some embodiments, induction can occur by adding an induction agent (such as a molecule, light, or heat) to the sample comprising the tumor cells. Inducing can occur for at least about any of 30 min, 1, 2, 3, 4, 8, 12, 16, 20, 24, 28, 32, 36, 40, 44, 48, or 72 hours or more. Inducing can occur for at most about any of 30 min, 1, 2, 3, 4, 8, 12, 16, 20, 24, 28, 32, 36, 40, 44, 48, or 72 hours. In some embodiments, the inducing step occurs for about 4 to about 48 hours, such as about 4 to about 8 hours, about 12 to about 48 hours, about 24 to about 48 hours, or about 12 to about 24 hours. In some instances, induction occurs for about 24 hours.

Induction can occur after the step of contacting the tumor cells with the cell-killing agent, or it can occur at the same time as the step of contacting the tumor cells with the cell-killing agent. Even if induction occurs at the same time as the contacting step, there may still be a silent phase (e.g., of about 4 hours) due to the time delay of transcription and translation involved in induction. In some embodiments, the contacting step occurs before the inducing step, such as at least about any of 30 min, 1, 2, 3, 4, 8, 12, 16, 20, 24, 28, 32, 36, 40, 44, 48, or 72 hours or more before the inducing step. In some embodiments, the contacting step occurs about 4 to about 48 hours before the inducing step, such as about 4 to about 8 hours, about 24 to about 48 hours, about 4 to about 24 hours, or about 12 to about 24 hours before the inducing step. In some embodiments, the contacting step occurs for at least about 24 hours prior to the inducing step. In some embodiments, the contacting step occurs for about 24 to about 48 hours prior to the inducing step. In some embodiments, the contacting step occurs for up to about 6 days (e.g., about any of 1, 2, 3, 4, 5, or 6 days) prior to the inducing step.

In some embodiments, the contacting step occurs after the inducing step, such as at least about any of 30 min, 1, 2, 3, 4, 8, 12, 16, 20, 24, 28, 32, 36, 40, 44, or 48 hours or more after the inducing step. In some embodiments, the contacting step occurs about 2 to about 48 hours (e.g., about 12 to about 24 hours) after the inducing step.

The timing of induction (i.e., defining the length of the silent phase) can depend on varying factors, such as the time to it takes for cell-killing agents to kill cells, the type of cell culture conditions (such as monolayer versus spheroid), the ratio of effector cells to tumor cells, the cell-killing agent's mechanism of action, and the total number of tumor cells. For example, cell-mediated killing using NK cells may occur relatively quickly compared to, for example, unstimulated PBMCs, and therefore waiting longer for induction may result in more non-specific cell-killing. As another example, unstimulated PBMC's may have a longer silent phase before induction since there may be some lag time required to activate the unstimulated T-cells. In another example, cell mediated killing under spheroid conditions may have a longer silent phase before induction than monolayer. In another example, cell-mediated killing with more effector cells may have a shorter silent phase before induction. In another example, different antibodies can have different mechanisms of action (e.g., some use ADCC, some activate T-cells via their CD3 binding sites). In some instances, using more tumor cells in the methods of the disclosure may mean that a longer silent phase before induction is needed.

In some instances, the tumor cells can be contacted with a cell-killing agent and an induction agent simultaneously. This may result in about a 4 hour silent phase in which cell killing can occur but the reporter protein has not yet been expressed (due to the time lag for induction). In some instances, the tumor cells can be contacted with a cell-killing agent and an induction agent sequentially. When the tumor cells are contacted with a cell-killing agent before an induction agent, the cell-killing agent can be contacted to the tumor cells at least about any of 30 min, 1, 2, 3, 4, 8, 12, 16, 20, 24, or 48 hours or more before the induction agent is added to the tumor cells. The cell-killing agent can be contacted to the tumor cells at least about any of 1, 2, 3, 4, 5, 6, 7, or 8 or more days before the induction agent is added to the tumor cells.

Induction of expression of the reporter protein can result in an increase in expression of the reporter protein by at least about any of 2-fold, 4-fold, 6-fold, 8-fold, 10-fold, 20-fold, 24-fold, 60-fold. 80-fold, 100-fold, 120-fold, 200-fold, 300-fold, 400-fold, 500-fold, 600-fold, 700-fold, 800-fold or 900-fold or more.

After the nucleic acid encoding the reporter protein has been induced (i.e., starting the expression phase), the reporter protein is produced. The reporter protein can be secretable. Secretable reporter proteins can be secreted outside of the cell and into the media and/or biological solution in which the cell is growing. Secretable reporter proteins can be secreted through a cell's normal secretory pathway (i.e., including rough endoplasmic reticulum, Golgi, and vesicles).

### Methods of Determining the Amount of Reporter Protein

The disclosure provides for methods of determining the amount of reporter protein (secreted or non-secreted) produced from the tumor cells. The step of determining the amount of reporter protein can comprise detecting the presence or absence of the reporter protein. The presence or absence of reporter protein can be detected by any suitable method. Exemplary methods for detecting the reporter protein can include, but are not limited to, detecting fluorescence of the reporter protein, detecting luminescence of the reporter protein, detecting RLU of the reporter protein, detecting the protein using a microplate reader (i.e., GloMax Discover Microplate reader), detecting using western blot, detecting using mass spectrometry, ELISA, FISH, PCR, and the like.

Luciferase can be detected by any suitable method. Commercial methods exist for detection of luciferase (e.g., Pierce™ Firefly Luciferase Glow Assay Kit. Sigma-Aldrich® Luciferase Reporter Gene Detection Kit). The mechanism to detect luciferase comprises release of light by bioluminescence of luciferase. This mechanism involves the oxidation of a substrate, i.e., a luciferin, in the presence of adenosine triphosphate (ATP) and oxygen to produce adenosine monophosphate (AMP), pyrophosphate, and carbon dioxide.

Detection of a reporter protein can comprise adding reagents to permit measurement of the enzyme activity of the reporter protein. Exemplary reagents may include, but are not limited to, free radical scavengers such as dithiothreitol (DTT), cytidine nucleotides, AMP, pyrophosphate, coenzyme A, chelating agents such as ethylene diaminetetraacetic acid (EDTA), detergents such as Triton® N-101 (nonylphenoxypolyethoxyethanol), buffers such as HEPES, N-[2-hydroxyethyl] piperazine-N¹-[2-ethane sulfonic acid], and protease inhibitors such as phenylacetic acid (PAA) and oxalic acid (OA).

Reporter protein (i.e., luciferase) catalyzed photon emission as disclosed by the methods and compositions of the disclosure can be detected for more than at least about any of 5, 10, 20, 30, 40, 50, or 60 or more minutes. The reporter protein can be detected for more than at least about any of 4, 8, 12, 16, 20, 24, 28, 32, 36, 40, 44, 48, 72, 96, 120, or 144 hours or more.

Determining the amount of the secretable reporter protein can comprise detection of the secretable reporter protein by sampling the media in which the cells are growing at various time points in order to continuously monitor reporter protein product (i.e., and subsequently cell-killing) in real-time. Media samples can be taken and analyzed at about any of 5, 10, 20, 30, 40, 50, and/or 60 or more minutes after the start of induction. Media samples can be taken and analyzed at about any of 2, 4, 8, 12, 16, 20, 24, 28, 32, 36, 40, 44, 48, 72, and/or 96 hours or more after the start of induction. Media samples can be taken and analyzed at 1 day, 2 days, 3 days, 4 days, 5 days, 6 days, or 7 or more days after the start of induction. In some instances, the sample is taken and analyzed 4 hours after induction. In some instances, the sample is taken 8 hours after induction. In some instances, the sample is taken 12 hours after induction. In some instances, the sample is taken 24 hours after induction. Any sample time point can be compared with any other sample time point. If the level of secreted luciferase at a given time point has decreased compared to an earlier time point, then it may indicate an increase in cell-killing by a cell-killing agent.

Multiple samples can be taken over time. Samples can be taken about every 1, 5, 10, 20, 30, 40, 50, or 60 or more minutes. Samples can be taken about every 1, 2, 4, 8, 12, 16, 20, 24 or more days. Samples can be taken about every 1, 2, 4, 8, 12, 16, 20, 24, 48, or more hours. Samples can be taken about every 1, 2, 3, 4, 5, 6, 7, or more days. In some instances, using GFP as an intracellular marker, samples may not need to be taken from the media or culture system in order to perform real time monitoring of reporter protein.

Detection of a non-secretable reporter protein can be done with any suitable method. Exemplary methods for detecting a non-secretable reporter protein include, but are not limited to, fluorescence imaging, western blot, mass spectrometry, and fluorescence activated cell sorting, immunocytochemistry (antibodies to marker proteins), gene arrays, and PCR (tests for mRNA characteristic of stem cells), and the like. Detection can occur without lysing the cells.

In some embodiments, multiple detection methods can be used to detect reporter proteins. For example, if the cells comprise more than one type of reporter protein (e.g., a secretable reporter, such as luciferase, and an intracellular reporter protein, such as a fluorescent protein), then the different reporter proteins can be detected with different detection methods suitable for each type of reporter protein. For example, cells can be imaged (i.e., such as with a Nikon Ellipse TE2000-U microscope) to detect intracellular GFP, and media samples can be taken to detect secreted luciferase (e.g., using a GloMax Discover Microplate Reader).

Determining the amount of reporter protein can comprise correlating the amount of detected reporter protein to the amount of cell survival or cell death. For example, the reporter protein can correlate with the amount of cell-killing occurring during the silent phase of the methods of the disclosure (i.e., by one or more cell-killing agents). The amount of reporter protein can negatively correlate with the effectiveness of the cell-killing agent. In other words, the more reporter protein detected, the more the cells are secreting the reporter protein, and therefore, the less cell-killing occurring. In some instances, the amount of reporter positively correlates with the number of live cells in the sample (i.e., those that weren't killed). The amount of reporter protein may correlate only with the number of cells that express the reporter protein. The amount of reporter protein may not have a relationship with the number of cells in the co-culture that do not express the reporter protein.

### Methods Comprising Constitutive Promoters

In some embodiments, the disclosure provides for methods for evaluating the effect of a cell-killing agent on a population of tumor cells, wherein the tumor cells constitutively express a reporter protein of the disclosure. Constitutive promoters initiate continual gene product production under most growth conditions Constitutive promoters can include the cauliflower mosaic virus (CMV), human Ubiquitin C promoter (UBC), human elongation factor 1α promoter (EF1A), mouse phosphoglycerate kinase 1 promoter (PGK), simian virus 40 (SV40), promoters obtained from the genomes of viruses such as polyoma virus, fowlpox virus, adenovirus (such as Adenovirus 2), bovine papilloma virus, avian sarcoma virus, cytomegalovirus, a retrovirus, and hepatitis-B virus, and the like.

A nucleic acid encoding a reporter protein operably linked to any promoter (i.e., a constitutive promoter) can be introduced into tumor cells (e.g., by transfection, transduction, or electroporation). The nucleic acid can express the secretable reporter protein which is secreted from the cells into the media in which the cells are growing. The reporter protein can be detected in the media at a first time point. The conditioned media can be replaced with fresh media in which there is no secreted reporter protein. Replacement acts as a way to “reset” the amount of secreted reporter protein in the media. Over time, the alive tumor cells will continue to express the reporter protein and secrete it into the media. The reporter protein can be detected in the replaced media at a second time point. The first and second time points can be compared to each other to determine how much cell-killing has occurred over time.

The media can be replaced any number of times. For example, media can be replaced at least 1, 2, 3, 4, 5, 6, 7, 8 or 9 or more times. In some embodiments, media can be replaced at most 1, 2, 3, 4, 5, 6, 7, 8 or 9 or more times. Media can be replaced after about 2 hours, 4 hours, 6 hours, 8 hours, 10 hours, 12 hours, 14 hours, 16 hours, 18 hours, 20 hours, 1 day, 2 days, 3 days, 4 days, 5 days, 6 days, or 7 days or more after the previous media replacement.

Any number of time points can be taken. Time points can be taken at least 1, 2, 3, 4, 5, 6, 7, 8, 9 or 10 or more times per media replacement cycle. Time points can be taken at most 1, 2, 3, 4, 5, 6, 7, 8, 9 or 10 times per media replacement cycle. In some instances, one time point is taken per media replacement cycle.

### Compositions

Any of the compositions described herein can be used in any of the methods of the disclosure.

### Cells

The disclosure provides for compositions comprising tumor cells (e.g., “inducible reporter tumor cell” comprising a nucleic acid encoding a reporter protein under control of an inducible promoter). Tumor cells can be primary tumor cells. Primary tumor cells can comprise tumor material obtained from a subject having cancer. Primary tumor cells can be obtained from tumor tissue samples, for example, tissue obtained by surgical resection and tissue obtained by biopsy (e.g., by a core biopsy or a fine needle biopsy.) Primary tumor cells can comprise tumor material from patient derived xenograft which are created when cancerous tissue from a patient's primary tumor is implanted directly into an immunodeficient mouse.

In some embodiments, there is provided a tumor cell (or a composition of tumor cells) comprising a nucleic acid encoding a luciferase and a GFP under the same control of an inducible promoter (e.g., TetOn). In some embodiments, the nucleic acid encoding GFP and the nucleic acid encoding luciferase are connected by IRES, or a nucleic acid encoding a self-cleaving 2A peptide, such as P2A, T2A, E2A, F2A, BmCPV 2A, BmIFV 2A. In some embodiments, there is provided a tumor cell (or a composition of tumor cells) comprising a nucleic acid from upstream to downstream: an inducible promoter (e.g., TetOn promoter)—nucleic acid encoding a first reporter protein (e.g., luciferase)—IRES or nucleic acid encoding a self-cleaving 2A peptide (e.g., P2A, T2A, E2A, F2A, BmCPV 2A, or BmIFV 2A)—nucleic acid encoding a second reporter protein (e.g., EGFP). In some embodiments, there is provided a tumor cell (or a composition of tumor cells) comprising a nucleic acid from upstream to downstream: TetOn promoter—nucleic acid encoding luciferase (snLuc)—nucleic acid encoding P2A—nucleic acid encoding EGFP (hereinafter referred to as “Tet-on snLuc-GFP construct”). In some embodiments, such nucleic acids are contained within a lentiviral vector. See, Example 1. In some embodiments, the inducible promoter is induced by doxycycline.

In some embodiments, there is provided a lentiviral vector comprising an inducible promoter (e.g., TetOn promoter)—nucleic acid encoding a first reporter protein (e.g., luciferase)—IRES or nucleic acid encoding a self-cleaving 2A peptide (e.g., P2A, T2A, E2A, F2A, BmCPV 2A, or BmIFV 2A)—nucleic acid encoding a second reporter protein (e.g., EGFP).

Primary tumor cells and/or tumor cells lines can comprise cells from any tumor that is epithelial in origin. For example, primary tumor cells and/or tumor cell lines can comprise cells from breast, ovary, endometrium, cervix, colon, lung, pancreas, eosophagus, prostate, small bowel, rectum, uterus or stomach; and squamous cell carcinomas, which may have a primary site in the lungs, oral cavity, tongue, larynx, eosophagus, skin, bladder, cervix, eyelid, conjunctiva, and the like. Primary tumor cells and/or tumor cell lines can comprise cells from malignancies of solid organs including carcinomas, sarcomas, melanomas and neuroblastomas. Primary tumor cells and/or tumor cell lines can comprise tumor cells from blood-borne (ie, dispersed) malignancy such as a lymphoma, a myeloma or a leukemia. Tumor cells can be part of a tumor cell line. Tumor cell lines comprise immortalized tumor cells. An immortalized cell, as used herein, can refer to a cell capable of growing in culture for more than 15 passages. The term passage number refers to the number of times that a cell population has been removed from the culture vessel and undergone a subculture (passage) process, in order to keep the cells at a sufficiently low density to stimulate further growth. Exemplary tumor cell lines can include LnCaP cells, MDA-MB-231 cells, MCF-7 cells, MDA-MB-468 cells, and SK-BR-3 cells, etc. In some embodiments, tumor cells are cultured in 2D monolayer. In some embodiments, tumor cells are cultured as a 3D spheroid.

The number of tumor cells that can be cultured in the compositions or methods of the disclosure can range from about 500 tumor cells to about 100,000 tumor cells, such as any of from about 500 tumor cells to about 1,000 tumor cells, from about 1,000 tumor cells to about 50,000 tumor cells, from about 10,000 tumor cells to about 50,000 tumor cells, from about 1,000 tumor cells to about 20,000 tumor cells, from about 1,000 tumor cells to about 15,000 tumor cells, from about 1,000 tumor cells to about 10,000 tumor cells, from about 1,000 tumor cells to about 5,000 tumor cells, from about 5,000 tumor cells to about 20,000 tumor cells, from about 5,000 tumor cells to about 15,000 tumor cells, from about 5.000 tumor cells to about 10,000 tumor cells, from about 10,000 tumor cells to about 20,000 tumor cells, from about 10,000 tumor cells to about 15,000 tumor cells, or from about 15,000 tumor cells to about 20,000 tumor cells. In some instances, the number of tumor cells is about 5,000 cells. In some instances, the number of tumor cells is about 10,000 cells. In some embodiments, the number of tumor cells is about 20,000 cells. In some embodiments, the number of tumor cells is about 15,000 cells.

Tumor cells can be co-cultured with one or more additional populations of cells (e.g., such as in a 3D spheroid configuration). Tumor cells can be cultured with at least 1, 2, 3, 4, 5, 6, 7, 8, or 9, or more additional populations of cells. Tumor cells can be cultured with at most 1, 2, 3, 4, 5, 6, 7, 8, or 9 additional populations of cells. In some instances, tumor cells of the disclosure are cultured with one additional cell population, e.g., fibroblasts. In some embodiments, the additional population of cells are tumor cells. In some embodiments, the additional population of cells are non-tumor cells.

The additional population(s) of cells can comprise non-tumor cells. For example, non-tumor cells can be tumor microenvironment promoting cells. In the context of cancer, the tumor microenvironment can be comprised of both malignant and non-malignant cells. While transforming or oncogenic alterations in the malignant cells can underlie unregulated growth and tumor progression, non-malignant cells and the tumor microenvironment, which results from the juxtaposition of malignant and non-malignant cells, may affect tumor initiation. Non-malignant cells and the tumor microenvironment can be relevant to tumor progression and maintenance of conditions that support genetic instability and elevated mutation frequencies. Non-malignant cells that function normally to support inflammatory and immune response within a tumor microenvironment may be capable of contributing to tumor progression, for example, by producing mediators that directly or indirectly support growth and viability of malignant cells within the tumor, or by producing mediators that directly or indirectly inhibit the growth and viability of malignant cells, or by inhibiting responses that would otherwise impede tumor progression. The tumor microenvironment may also influence accessibility of a tumor to therapeutic intervention by altering drug metabolism or pharmacokinetics at the tumor site and/or contributing to the development of drug resistance. Exemplary non-tumor cells (i.e., tumor microenvironment promoting cells) can include stromal cells, fetal fibroblast cells, bone marrow fibroblast cells, endothelial cells, tumor associated macrophage, myeloid-derived suppressive cells, or any combination/variants thereof. In some instances, the non-tumor cell (i.e., tumor microenvironment promoting cell) is a fibroblast cell.

The number of non-tumor cells that can be cultured in a composition or method of the disclosure can range from about 500 non-tumor cells to about 100,000 non-tumor cells, such as any of from about 500 non-tumor cells to about 1,000 non-tumor cells, from about 1,000 non-tumor cells to about 50,000 non-tumor cells, from about 10,000 non-tumor cells to about 50,000 non-tumor cells, from about 1,000 non-tumor cells to about 20,000 non-tumor cells, from about 1,000 non-tumor cells to about 15,000 non-tumor cells, from about 1.000 non-tumor cells to about 10,000 non-tumor cells, from about 1,000 non-tumor cells to about 5,000 non-tumor cells, from about 5,000 non-tumor cells to about 20,000 non-tumor cells, from about 5,000 non-tumor cells to about 15,000 non-tumor cells, from about 5,000 non-tumor cells to about 10,000 non-tumor cells, from about 10.000 non-tumor cells to about 20,000 non-tumor cells, from about 10,000 non-tumor cells to about 15,000 non-tumor cells, or from about 15,000 non-tumor cells to about 20,000 non-tumor cells. In some instances, the number of non-tumor cells is about 5,000 cells. In some instances, the number of non-tumor cells is about 6,000 cells. In some instances, the number of non-tumor cells is about 10,000 cells.

Compositions and methods of the disclosure can include tumor cells and non-tumor cells in varying ratios. The ratio of tumor cells to non-tumor cells in a culture can be at least about any of 1:1, 2:1, 3:1, 4:1, 5:1, 6:1, 7:1, 8:1, or 9:1 or more. The ratio of tumor cells to non-tumor cells in a culture can be at most about any of 1:1, 2:1, 3:1, 4:1, 5:1, 6:1, 7:1, 8:1, or 9:1. The ratio of non-tumor cells to tumor cells in a culture can be at least about any of 1:1, 2:1, 3:1, 4:1, 5:1, 6:1, 7:1, 8:1, or 9:1, or more. The ratio of non-tumor cells to tumor cells in a culture can be at most about any of 1:1, 2:1, 3:1, 4:1, 5:1, 6:1, 7:1, 8:1, or 9:1. In some instances, the ratio of tumor cells to non-tumor cells is about 1:1.

Tumor cells and/or tumor co-cultures (i.e., comprising tumor and non-tumor cells) can be grown or incubated in any suitable topology. For example, tumor cells and/or tumor co-cultures (i.e., with non-tumor cells) can be grown or incubated in a 2D monolayer or a 3D spheroid. In 2D monolayer cell culture, tumor cells can be co-cultured with a “feeder layer” of fibroblasts or other cells to supply the tumor cells (such as primary tumor cells) with nutrients and other factors.

A three-dimensional (3D) spheroid can comprise an aggregation of tumor cells comprising a small mass, or lump of tumor cells. It is noted that the term “spheroid” does not imply that the aggregate is a geometric sphere. The aggregate may be highly organized with a well-defined morphology or it may be an unorganized mass. The spheroid may include a single cell type or more than one cell type (i.e., population of cells). The cells may be primary isolates, or a permanent cell line, or a combination of the two. A spheroid can comprise mammospheres, organoids, and organotypic cultures. Tumor cell spheroids can be grown or incubated in plates, in capillaries, in microfluidics, in 3D structures, and the like.

Tumor spheroids can be less than about 5 cm, less than about 4 cm, less than about 3 cm, less than about 2 cm, less than about 1 cm, less than about 5 mm, less than about 2.5 mm, less than about 1 mm, less than about 500 μm, less than about 100 μm, less than about 50 μm, less than about 25 μm, less than about 10 μm, or less than about 5 μm in diameter. In some instances, the tumor spheroids have a diameter of about 10 μm to about 500 μm. In some instances, the tumor spheroids have a diameter of about 40 μm to about 100 μm.

### Reporter Proteins

The disclosure provides for tumor cells comprising nucleic acids encoding reporter proteins. A reporter protein is a protein that acts as a readout for any change occurring in cells (i.e., such as enzymatic changes, morphological changes, cell-signaling changes, or ADCC, and the like). Reporter proteins can include fragments, variants and recombinant forms of a reporter protein.

A reporter protein can be secretable. A secretable reporter protein can refer to a reporter protein that can be secreted from the cell in which it is expressed into an extracellular location. The extracellular location may be internal or external to the organism or cell depending on the identity of the organism or cell. The extracellular location includes within its scope the medium in which a cell expressing the reporter protein is being cultured in vitro. A secretable reporter protein can comprise any modified and recombinant forms thereof. For example, a secretable reporter protein can be a protein that is not secretable in its native form, but has been modified to become sercretable (i.e., through modification with a signal peptide, i.e., a secretion signal tag). A “signal peptide” can refer to a leader sequence ensuring entry into the secretory pathway. A signal peptide can be a short amino acid sequence that directs newly synthesized secretory or membrane proteins to and through cellular membranes such as the endoplasmic reticulum. A secretion signal peptide can be a homologous, heterologous, hybrid, and synthetic signal peptide. Heterologous secretion signal sequences are generally either associated in nature with the heterologous gene being expressed, or are derived from another, non-mammalian gene. Hybrid signal sequences generally comprise elements of two different signal sequences.

A secretable reporter protein can be generated by fusing a secretory signal sequence to the wild-type reporter protein using standard recombinant DNA methodology familiar to one of skill in the art. The secretory signal sequence may be positioned at the N-terminus of the desired reporter protein but can be placed at any position suitable to allow secretion of the reporter protein. Suitable secretory signal sequences can include signal sequences or derivatives of signal sequences of known secretory proteins. A variety of secretory proteins have been identified. They include but are not limited to certain growth factors such as fibroblast growth factors 4-6, epidermal growth factor, and lymphokines such as interleukins 2-6.

Exemplary secretable reporter proteins can include Oplophorus luciferase, beetle luciferase, Renilla luciferase, Metridia luciferase, Gaussia luciferase, NANOLUC luciferase, secretable fluorescent protein (e.g., secretable GFP, YFP, CFP, RFP), secreted alkaline phosphatase, secretable beta-galactosidase, proteins associated with exosomes, proteins associated with secreted vesicles, or any combination thereof. In some instances, the reporter protein is a secretable luciferase. In some instances, the reporter protein is a protein that has similar sensitivity and/or dynamic range as secreted luciferase. In some instances, the reporter protein is secretable GFP or EGFP.

A reporter protein may be non-secretable. A non-seretable reporter protein can refer to a reporter protein that upon expression is retained within the cell or cell membrane rather than secreted into the extracellular medium (i.e., intracellular). A non-secretable reporter protein comprises any modified and recombinant polypeptide or fragment forms thereof. Exemplary non-secretable reporter proteins can include, but are not limited to, fluorescent proteins GFP, BFP, CFP, YFP, EGFP, EYFP, Venus, Citrine, phiYFP, copGFP CGFP, ECFP, Cerulean, CyPet, T-Sapphire, Emerald, YPet, AcGFP1, AmCyan, AsRed2, dsRed, dsRed2, dsRed-Express, EBFP, HcRed, ZsGreen, ZsYcllow, J-Red, TurboGFP, Kusabira Orange, Midoriishi Cyan, mOrange, DsRed-monomer, mStrawberry, mRFPI, tdTomato, mCherry, mPlum, and mRaspberry, lacZ, beta-galactosidase, non-secretable luciferase, chloramphenicol acetyltransferase, and the like. In some instances, the reporter protein is a non-secretable GFP.

The nucleic acid encoding the reporter protein of the disclosure can be present on a vector (e.g., a plasmid, an artificial chromosome, a BAC, and the like). The vector components can generally include, but are not limited to, one or more of the following: a signal sequence, an origin of replication, one or more marker genes, and enhancer element, a promoter, and a transcription termination sequence.

A vector for use in a eukaryotic host may comprise an insert that encodes a signal sequence or other polypeptide having a specific cleavage site at the N-terminus of the mature protein or polypeptide. The heterologous signal sequence selected may be one that is recognized and processed (i.e., cleaved by a signal peptidase) by the tumor cell. The heterologous signal sequence selected may not be one that is recognized and processed (i.e., cleaved by a signal peptidase) by the tumor cell. In mammalian cell expression, mammalian signal sequences as well as viral secretory leaders, for example, the herpes simplex virus glycoprotein D (gD) signal, can be available. The DNA for such precursor region can be ligated in reading frame to DNA encoding the reporter protein of the disclosure.

Expression and cloning vectors may contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media.

Expression and cloning vectors can comprise a promoter that is recognized by the host organism and is operably linked to the nucleic acid encoding the reporter protein. Nucleic acid is “operably linked” when it is placed into a functional relationship with another nucleic acid sequence. For example, DNA for a presequence or secretory leader can be operably linked to DNA for a polypeptide if it is expressed as a preprotein that participates in the secretion of the polypeptide; a promoter or enhancer can be operably linked to a coding sequence if it affects the transcription of the sequence; or a ribosome binding site can be operably linked to a coding sequence if it is positioned so as to facilitate translation. Generally, “operably linked” means that the DNA sequences being linked are contiguous, and/or contiguous an in reading frame. Enhancers may not have to be contiguous. Linking is accomplished by ligation at convenient restriction sites. If such sites do not exist, the synthetic oligonucleotide adaptors or linkers are used in accordance with conventional practice.

A promoter can refer to a DNA regulatory region capable of binding RNA polymerase in a cell and initiating transcription of a downstream (3′ direction) coding sequence. A promoter may be bounded at its 3′ terminus by the transcription initiation site and extends upstream (5′ direction) to include the minimum number of bases or elements necessary to initiate transcription at levels detectable above background. Within the promoter sequence there may be a transcription initiation site, and/or protein binding regions responsible for the binding of RNA polymerase. Eukaryotic promoters may contain TATA boxes and CAT boxes. Various promoters, including inducible promoters, may be used to drive expression.

The promoter operably linked to the nucleic acid encoding the reporter protein (or KO construct such as CRISPR/Cas) can be inducible. Inducible promoters are those that control the expression of the reporter protein based on the presence of an induction agent (i.e., molecule). Exemplary inducible promoters can include estrogen-inducible, estradiol-inducible, ACE1 promoter, IN2 promoter, tetracycline-inducible promoter (e.g., TetOn), tissue-specific promoters (i.e., myosin heavy chain promoter for muscle specific expression, lysosomal acid lipase promoter, amylase promoter, folylpoly-gamma-glutamate synthetase promoter, neural restrictive silencer element, HGH promoter, prolactin promoter, and alpha1 (VI) collagen promoter), cell type specific promoters (i.e., E2F 1 promoter; a cyclin A promoter; a cyclin B promoter; a cyclin C promoter; a cyclin D promoter; a cyclin E promoter; and the like), developmental stage-specific promoters (i.e., notch, numb, homeotic genes, murine homeobox promoters), promoters controlled by the cell cycle, promoters controlled by Circadian rhythm, and promoters whose activity is increased (e.g., activated) or decreased (e.g., suppressed) in response to an external or internal signal, or any combination thereof. Exemplary induction agents (i.e., molecules) can include tetracycline, doxycycline, estrogen receptor, and cumate, and the like. In some embodiments, the inducible promoter is a TetOn system. Other exemplary methods of inducing expression can include exposing the tumor cells to light or heat.

In some embodiments, the promoter operably linked to the nucleic acid encoding the reporter protein (or KO construct such as CRISPR/Cas targeting PD-L1) can be constitutive. Constitutive promoters allow heterologous genes (also referred to as transgenes) to be expressed constitutively in cells. Exemplary constitutive promoters contemplated herein include, but are not limited to, cytomegalovirus immediate-early promoter (CMV), human elongation factors-1alpha (hEF1α), ubiquitin C promoter (UbiC), phosphoglycerokinase promoter (PGK), simian virus 40 early promoter (SV40), chicken β-Actin promoter coupled with CMV early enhancer (CAGG), a Rous Sarcoma Virus (RSV) promoter, a polyoma enhancer/herpes simplex thymidine kinase (MC1) promoter, a beta actin (β-ACT) promoter, a “myeloproliferative sarcoma virus enhancer, negative control region deleted, d1587rev primer-binding site substituted (MND)” promoter. The efficiencies of such constitutive promoters on driving transgene expression have been widely compared in a huge number of studies. In some embodiments, the promoter operably linked to the nucleic acid encoding a KO construct (e.g., CRISPR/Cas) against endogenous PD-L1 is CMV.

Transcription of a DNA encoding the reporter protein of the disclosure may be increased by inserting an enhancer sequence into the vector. Enhancer sequences can include those from mammalian genes (globin, elastase, albumin, α-fetoprotein, and insulin), or eukaryotic cell viruses such as, the SV40 enhancer on the late side of the replication origin (100-270 bp), the cytomegalovirus early promoter enhancer, the polyoma enhancer on the late side of the replication origin, and adenovirus enhancers. The enhancer may be spliced into the vector at a position 5′ or 3′ to the polypeptide encoding sequence.

Expression vectors used in eukaryotic tumor cells (yeast, fungi, insect, plant, animal, human, or nucleated cells from other multicellular organisms) can comprise sequences used for termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the polypeptide-encoding mRNA.

Expression vectors may have restriction sites to provide for the insertion of nucleic acid sequences encoding the reporter protein. A selectable marker operative in the expression tumor cell may be present. Expression vectors may be prepared comprising a transcription initiation region, a coding sequence or fragment thereof, and a transcriptional termination region.

In some instances, the expression vector can include a coding sequence that encodes a viral protein. The viral protein may be a component of a viral vector, which may be used in viral transduction in order to express the nucleic acid encoding a reporter protein in a tumor cell of the disclosure. Exemplary viral vectors can include, but are not limited to, retroviral, e.g., lentiviral, vectors: adenoviral vectors; adeno-associated virus (AAV) viral vectors, feline immunodeficiency virus (FIV) vectors, rabies virus vectors, avian sarcoma leukosis virus (ASLV) vectors, or any combination thereof.

Vectors may encode one or more viral proteins, such as enzymes, e.g., polymerase, capsid proteins, envelope proteins, regulatory proteins, and the like. Vectors can be configured to carry sequences for incorporating foreign nucleic acid, for selection and/or for transfer of the nucleic acid into a tumor cell of the disclosure.

Polynucleic acid sequences encoding the reporter protein (or KO construct such as CRISPR/Cas targeting PD-L1) of the disclosure can be obtained using standard recombinant techniques. Desired polynucleic acid sequences may be isolated and sequenced from cells. Alternatively, polynucleotides can be synthesized using nucleotide synthesizer or PCR techniques. Once obtained, sequences encoding the polypeptides can be inserted into a recombinant vector capable of replicating and expressing the heterologous polynucleotides in tumor cells.

The nucleic acid encoding the reporter protein (or KO construct such as CRISPR/Cas targeting PD-L1) can be introduced into a tumor cell of the disclosure by any method. For example, the nucleic acid encoding the reporter protein (or KO construct such as CRISPR/Cas targeting PD-L1) can be introduced into a cell through retroviral or lentiviral transduction. Viral particles can be generated by co-expressing the virion packaging elements and the vector genome in a so-called producer cell, e.g., 293T human embryonic kidney cells. These cells may be transiently transfected with a number of nucleic acids (e.g., viral components). Other exemplary methods for introducing a nucleic acid encoding a reporter protein can include: transfection, transient transfection, stable transfection, electroporation, and the like.

Tumor cells of the disclosure can comprise any number of different nucleic acids encoding reporter proteins (or KO construct such as CRISPR/Cas targeting PD-L1). For example, tumor cells can comprise at least 1, 2, 3, 4, 5, 6, 7, 8, or 9, or more different nucleic acids encoding different reporter proteins. Tumor cells can comprise at most 1, 2, 3, 4, 5, 6, 7, 8, or 9 different nucleic acids encoding different reporter proteins. Tumor cells can comprise a first nucleic acid encoding a first reporter protein (e.g., luciferase) and one or more further nucleic acids encoding a different reporter protein (e.g., GFP). In some instances, the cell can comprise a first reporter nucleic acid comprising a secretable reporter protein (e.g., a secretable luciferase) and the cell can comprise a second nucleic acid encoding an intracellular reporter protein (e.g., a fluorescent protein).

When tumor cells of the invention express two reporter proteins, the two reporter proteins can be the same or different, e.g., one is non-secretable GFP and one is secretable GFP, or one is non-secretable GFP and one is secretable luciferase. The nucleic acids encoding the two reporter proteins can be on the same vector or on different vectors. The nucleic acids encoding the two reporter proteins can be under control of the same promoter on the same vector (e.g., linked via IRES, or nucleic acid encoding self-cleaving 2A peptide such as P2A, T2A, E2A, F2A, BmCPV 2A, BmIFV 2A in between), the same promoter on different vectors (e.g., both TetOn), different promoters on the same vector, or different promoters on different vectors (e.g., one is inducible, one is constitutive). When present on different vectors, the vectors can be transduced into tumor cells simultaneously or sequentially. In some embodiments, the nucleic acids encoding the two reporter proteins (luciferase and GFP) are under control of the same inducible promoter (e.g., TetOn).

### Cell-Killing Agents

The disclosure provides for compositions comprising cell-killing agents. The cell-killing agent can directly interact with the tumor cell. The cell-killing agent can indirectly interact with the tumor cell. Indirect interaction with a tumor cell can refer to, for example, a cell-killing agent that interacts with an immune cell to modulate the immune cell's ability to kill the tumor cell. As used herein, the term “cell-killing agent” encompasses direct and indirect cell-killing agents. For example, the cell-killing agent can refer to the combination of an antibody (i.e., an immunomodulatory antibody) and an immune cell as described herein. In some embodiments, the cell-killing agent effect target specific killing (e.g., via ADCC, BiTE, etc.). In some embodiments, cell-killing agent effect non-specific killing, such as NK cells which can perform nonspecific killing via killer-cell immunoglobulin-like receptor (KIR) recognition of MHC on tumor cells, in the absence of antibody targeting.

The cell killing agent can be a cytotoxin. A cytotoxin can be any agent that is detrimental to cells. Examplary cytotoxins can include, but are not limited to, taxol, cytochalasin B, gramicidin D, ethidium bromide, emetine, mitomycin, etoposide, tenoposide, vincristine, vinblastine, colchicin, doxorubicin, daunorubicin, dihydroxy anthracinedione, mitoxantrone, mithramycin, actinomycin D, l-dehydrotestosterone, glucocorticoids, procaine, tetracaine, lidocaine, propranolol, and puromycin and analogs or homologs thereof. Other toxins include, for example, ricin, CC-1065 and analogues, the duocarmycins. Still other toxins include diptheria toxin, and snake venom (e.g., cobra venom), DNA, RNA, RNAi, microRNAs, molecules that induce apoptosis, caspase activators, cytokine activators, and the like.

The cell-killing agent can be a cell. When the cell-killing agent is a cell, it may be referred to as an “effector cell.” An effector cell can participate in antibody-dependent cell mediated killing (ADCC), whereby an effector cell is able to kill a tumor cell via interaction with an antibody. A cell-killing agent can be any cell. A cell-killing agent can be an immune cell. Exemplary cell-killing immune cells (or effector cells) can include an NK cell, an NKT cell, a T cell, a CAR T cell, a monocyte, a neutrophil, a macrophage, a leukocyte, a lymphocyte, a T lymphocyte (such as a killer T cell (T_{c}, cytotoxic T lymphocyte, or CT), a helper T cell (Tₕ), a regulatory T cells (Treg), or a γδ T cell), a B lymphocyte, an eosinophil, a mast cell, a CD14+ cell, a dendritic cell, and a PBMC cell, or any combination thereof. When the cell-killing agent is an immune cell and can directly kill the tumor cell, such as a CAR-T cell, it can be used to detect if a patient has generated specific anti-cancer memory T-cells. In some embodiments, the effector cells are stimulated. In some embodiments, the effector cells are unstimulated.

When the cell-killing agent is a cell (i.e., an effector cell), the cell-killing agent can be incubated with tumor cells in varying ratios (E:T ratio). The ratio of tumor cells to cell-killing agent (i.e., effector cells) in a culture can be at least about any of 1:1, 2:1, 3:1, 4:1, 5:1, 6:1, 7:1, 8:1, or 9:1 or more. The ratio of tumor cells to cell-killing agent (i.e., effector cells) in a culture can be at most about any of 1:1, 2:1, 3:1, 4:1, 5:1, 6:1, 7:1, 8:1, or 9:1. The ratio of cell-killing agent (i.e., effector cells) to tumor cells in a culture can be at least about any of 1:1, 2:1, 3:1, 4:1, 5:1, 6:1, 7:1, 8:1, 9:1, 10:1, 15:1, 20:1, 25:1, or more. The ratio of cell-killing agent (i.e., effector cells) to tumor cells in a culture can be at most about any of 1:1, 2:1, 3:1, 4:1, 5:1, 6:1, 7:1, 8:1, 9:1, 10:1, 15:1, 20:1, or 25:1 In some instances, the ratio of cell-killing agent (i.e., effector cells) to tumor cells is about 3:1. In some instances, the ratio of cell-killing agent (i.e., effector cells) to tumor cells is about 1:1. In some embodiments, the ratio of cell-killing agent (i.e., effector cells) to tumor cells is about 9:1. In some instances, the ratio of cell-killing agent (i.e., effector cells) to tumor cells is about 25:1, 10:1, or 5:1.

The number of cell-killing agents (i.e., effector cells) that can be cultured in a composition or method of the disclosure can range from about 500 cells to about 100,000 cells, such as any of from about 1,000 cells to about 50,000 cells, from about 500 cells to about 1,000 cells, from about 10,000 cells to about 50,000 cells, from about 1,000 cells to about 30,000 cells, from about 1,000 cells to about 25,000 cells, from about 1,000 cells to about 20,000 cells, from about 1,000 cells to about 15,000 cells, from about 1,000 cells to about 10,000 cells, from about 1,000 cells to about 5,000 cells, from about 5,000 cells to about 30,000 cells, from about 5,000 cells to about 25,000 cells, from about 5,000 cells to about 20,000 cells, from about 5,000 cells to about 15,000 cells, from about 5,000 cells to about 10,000 cells, from about 10,000 cells to about 30,000 cells, from about 10,000 cells to about 25,000 cells, from about 10,000 cells to about 20,000 cells, from about 10,000 cells to about 15,000 cells, from about 15,000 cells to about 30,000 cells, from about 15,000 cells to about 25,000 cells, from about 15,000 cells to about 20,000 cells, from about 20,000 cells to about 30,000 cells, from about 20,000 cells to about 25,000 cells, or from about 25,000 cells to about 30,000 cells. In some embodiments, the number of effector cells is about 30,000 cells. In some instances, the number of effector cells is about 15,000 cells. In some instances, the number of effector cells is about 5,000 cells.

The cell killing agent can be an antibody. The antibody can comprise a heavy chain and a light chain. The heavy chain can comprise a V_{H} domain. The heavy chain may further comprise one or more constant domains, such as C_{H}1, C_{H}2, C_{H}3, or any combination thereof. The light chain can comprise a V_{L} domain, and may further comprise a constant domain, such as C_{L}. The heavy chain and the light chain can be connected to each other via a plurality of disulfide bonds. The antibody can comprise an Fc, such as an Fc fragment of the human IgG1, IgG2, IgG3, or IgG4. In some embodiments, the antibody does not comprise an Fc fragment. In some embodiments, the antibody has been inactivated or reduced for Fc function, such as by LALA mutations.

In some embodiments, the antibody is an antigen-binding fragment, such as any antigen-binding fragment format known in the art, e.g., an scFv, a VH, a VL, an scFv-scFv, an Fv, a Fab, a Fab′, a (Fab′)2, a minibody, a diabody, a domain antibody variant (dAb), a single domain antibody (sdAb) such as a camelid antibody (VHH) or a V_{NAR}, a fibronectin 3 domain variant, an ankyrin repeat variant, or other antigen-specific binding domains derived from other protein scaffolds.

The antibody can comprise a single polypeptide chain (e.g., scFv, or scFv-scFv). The antibody can comprise more than one (such as any of 2, 3, 4, or more) polypeptide chains. The polypeptide chain(s) may be of any length, such as at least about any of 10, 20, 50, 100, 200, 300, 500, or more amino acids long. In the cases of multi-chain antibodies, the nucleic acid sequences encoding the polypeptide chains may be operably linked to the same promoter or to different promoters.

The antibody can be a native antibody, such as monoclonal antibodies. Native antibodies are immunoglobulin molecules that are immunologically reactive with a particular antigen. The antibody can be an agonistic antibody. The antibody can be an antagonistic antibody. The antibody can be a monoclonal antibody. The antibody can be a polyclonal antibody. The antibody can be a human antibody, a humanized antibody, or a chimeric antibody. In some embodiments, the antibody is of non-human origin, e.g., mouse, rat, rabbit, goat, etc. antibody.

The antibody can be a monovalent antibody. The antibody can be a multivalent antibody, such as a divalent antibody or a tetravalent antibody. The antibody can be monospecific (e.g., anti-PD-1 antibody such as nivolumab, anti-HER2 antibody such as trastuzmab, or anti-PD-L1 antibody such as atezolizumab or durvalumab). The antibody can be multispecific (such as bispecific), such as an anti-HER2/anti-CD3 antibody, an anti-HER2/anti-CD47/anti-CD3 antibody, or an anti-PD-L1/anti-CD47/anti-CD3 antibody. Multispecific antibodies can have binding specificities for at least two different antigens or epitopes (e.g., bispecific antibodies have binding specificities for two antigens or epitopes).

Immune Checkpoint Molecule

In some embodiments, the antibody can specifically recognize an immune checkpoint molecule (such as anti-PD-1, anti-PD-L1, or anti-PD-L2 full-length antibody). Antibodies that act as checkpoint inhibitors can be referred to as an “immunomodulating agent.” Immune checkpoints are molecules in the immune system that either turn up (stimulatory molecules) or turn down a signal (inhibitory molecules). Immune checkpoint proteins can regulate and maintain self-tolerance and the duration and amplitude of physiological immune responses. Stimulatory checkpoint molecules can include, but are not limited to, CD27, CD40, OX40, GITR and CD137, which belong to tumor necrosis factor (TNF) receptor superfamily, as well as CD28 and ICOS, which belong to the B7-CD28 superfamily. Inhibitory checkpoint molecules include, but are not limited to, program death 1 (PD-1), Cytotoxic T-Lymphocyte-Associated protein 4 (CTLA-4), Lymphocyte Activation Gene-3 (LAG-3), T-cell Immunoglobulin domain and Mucin domain 3 (TIM-3, HAVCR2), V-domain Ig suppressor of T cell activation (VISTA, B7-H5), B7-H3, B7-H4 (VTCN1), HHLA2 (B7-H7), B and T Lymphocyte Attenuator (BTLA), Indoleamine 2,3-dioxygenase (IDO), Killer-cell Immunoglobulin-like Receptor (KIR), adenosine A2A receptor (A2AR). T cell immunoreceptor with Ig and ITIM domains (TIGIT), 2B4 (CD244) and ligands thereof. Numerous checkpoint proteins have been studied extensively, such as CTLA-4 and its ligands CD80 (B7-1) and CD86, and PD-1 (CD279) with its ligands PD-L1 (B7-H1, CD274) and PD-L2 (B7-DC, CD273).

The antibody specifically recognizing an immune checkpoint molecule can be immune checkpoint inhibitors (inhibitors of inhibitory immune checkpoint molecules) or activators of stimulatory immune checkpoint molecules. The antibody specifically recognizing an immune checkpoint molecule can be an activator of a stimulatory immune checkpoint molecule, such as an agonist antibody. e.g. anti-CD28, anti-OX40, anti-ICOS, anti-GITR, anti-4-1BB, anti-CD27, anti-CD40, anti-CD3, and anti-HVEM. The antibody specifically recognizing an immune checkpoint molecule can be an immune checkpoint inhibitor, such as inhibitors of PD-1 (CD279), PD-L1 (B7-H1, CD274), PD-L2 (B7-DC, CD273), LAG-3, TIM-3 (HAVCR2), BTLA, CTLA-4, TIGIT, VISTA (B7-H5), B7-H4 (VTCN1), CD160 (BY55), HHLA2 (B7-H7), 2B4 (CD244), CD73, B7-1 (CD80), B7-H3 (CD276), CD20, Her2, KIR, or IDO.

The antibody (i.e., cell-killing agent) recognizing an immune checkpoint molecule can be an immune checkpoint inhibitor. The immune checkpoint inhibitor can target immune cells (i.e., T cells.) The immune checkpoint inhibitor can target tumor cells. For example, in some cases, tumor cells can turn off activated T cells, when they attach to specific T-cell receptors. However, immune checkpoint inhibitors may prevent tumor cells from attaching to T cells so that T cells stay activated. The immune checkpoint inhibitor can be an antibody (such as antagonist antibody) that targets an inhibitory immune checkpoint protein (e.g., such as on an immune cell), including but not limited to, anti-CTLA-4, anti-TIM-3, anti-LAG-3, anti-KR, anti-PD-1 (e.g., nivolumab such as Opdivo®, Cemiplimab, or Pembrolizumab), anti-PD-L1 (e.g., Atezolizumab, Avelumab, or Durvalumab), anti-CD73, anti-B7-H3, anti-CD47, anti-BTLA, anti-VISTA, anti-A2AR, anti-B7-1, anti-B7-H4, anti-CD52, anti-IL-10, anti-IL-35, and anti-TGF-β. When an antibody targets a tumor cell (e.g., via CDC), it can be referred to as a direct cell-killing agent. When an antibody targets an immune cell (e.g., via ADCC), it can be referred to as an indirect cell-killing agent.

In some embodiments, the cell killing agent is an antibody that specifically recognizes a target cell (e.g., tumor cell) antigen, and/or an effector cell molecule (e.g., CD3). In some embodiments, the target antigen is a cell surface molecule (e.g., extracellular domain of a receptor/ligand). In some embodiments, the target antigen acts as a cell surface marker on a target cell (e.g., tumor cell) associated with a special disease state. The target antigens (e.g., tumor antigen, extracellular domain of a receptor/ligand) specifically recognized by the antigen-binding domain of the antibody may be antigens on a single diseased cell or antigens that are expressed on different cells that each contribute to the disease. The target antigens specifically recognized by the antigen-binding domain(s) may be directly or indirectly involved in the diseases.

Tumor Antigen

In some embodiments, the target cell antigen is a tumor antigen. Tumor antigens are proteins that are produced by tumor cells that can elicit an immune response, particularly T cell mediated immune responses. The selection of the targeted antigen of the invention will depend on the particular type of cancer to be treated. Exemplary tumor antigens include, for example, a glioma-associated antigen, BCMA (B-cell maturation antigen), carcinoembryonic antigen (CEA), β-human chorionic gonadotropin, alpha-fetoprotein (AFP), lectin-reactive AFP, thyroglobulin, RAGE-1, MN-CAIX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-la, p53, prostein, PSMA, HER2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor, and mesothelin. In some embodiments, the tumor antigen comprises one or more antigenic cancer epitopes associated with a malignant tumor. Malignant tumors express a number of proteins that can serve as target antigens for an immune attack. These molecules include but are not limited to tissue-specific antigens such as MART-1, tyrosinase and gp100 in melanoma and prostatic acid phosphatase (PAP) and prostate-specific antigen (PSA) in prostate cancer. Other target molecules belong to the group of transformation-related molecules such as the oncogene HER2/Neu/ErbB-2. Yet another group of target antigens is onco-fetal antigens such as carcinoembryonic antigen (CEA). In B-cell lymphoma, the tumor-specific idiotype immunoglobulin constitutes a truly tumor-specific immunoglobulin antigen that is unique to the individual tumor. B-cell differentiation antigens such as CD19, CD20 and CD37 are other candidates for target antigens in B-cell lymphoma.

In some embodiments, the tumor antigen is a tumor-specific antigen (TSA) or a tumor-associated antigen (TAA). A TSA is unique to tumor cells and does not occur on other cells in the body. A TAA is not unique to a tumor cell, and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development, when the immune system is immature, and unable to respond or they may be antigens that are normally present at extremely low levels on normal cells, but which are expressed at much higher levels on tumor cells. Non-limiting examples of TSA or TAA antigens include the following: differentiation antigens such as MART-1/MelanA (MART-I), gp 100 (Pmel 17), tyrosinase, TRP-1. TRP-2 and tumor-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5. MAGE-6, RAGE, NY-ESO, pl85crbB2, pl80erbB-3, c-met, nm-23H1, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS 1, SDCCAG16, TA-90\Mac-2 binding protein\cyclophilin C-associated protein, TAAL6, TAG72, TLP, and TPS.

In some embodiments, the tumor antigen is selected from the group consisting of Mesothelin, TSHR. CD19, CD123, CD22, CD30, CD171, CS-1, CLL-1, CD33, EGFRvIII, GD2, GD3, BCMA, Tn Ag, prostate specific membrane antigen (PSMA), ROR1, FLT3, FAP, TAG72, CD38, CD44v6, CEA, EPCAM, B7H3, KIT, IL-13Ra2, interleukin-11 receptor a (IL-11Ra), PSCA, PRSS21, VEGFR2, LcwisY, CD24, platelet-derived growth factor receptor-beta (PDGFR-beta), SSEA-4, CD20, Folate receptor alpha, ERBB2 (Her2/neu), MUC1, epidermal growth factor receptor (EGFR), NCAM, Prostase. PAP, ELF2M, Ephrin B2, IGF-I receptor. CAIX, LMP2, gp100, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, CLDN18.2, GPRC5D, CXORF61, CD97, CD179a, ALK, Polysialic acid, PLAC1, GloboH, NY-BR-1, UPK2, HAVCRL, ADRB3, PANX3, GPR20, LY6K, OR51E2, TARP, WT1, NY-ESO-1, LAGE-1a, MAGE-A1, legumain, HPV E6, E7, MAGE A1, ETV6-AML, sperm protein 17, XAGE1, Tie 2, MAD-CT-1, MAD-CT-2, Fos-related antigen 1, p53, p53 mutant, prostein, survivin and telomerase. PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, and IGLL1.

In some embodiments, the tumor antigen is HER2. In some embodiments, the antigen-binding domain specifically recognizing HER2 is derived from trastuzumab (e.g., Herceptin®), pertuzumab (e.g., Perjeta®), margetuximab, or 7C2. In some embodiments, the antigen-binding domain specifically recognizing HER2 comprises heavy chain CDRs, light chain CDRs, or all 6 CDRs of any of trastuzumab, pertuzumab, margetuximab, or 7C2. In some embodiments, the antigen-binding domain specifically recognizing HER2 comprises VH and/or VL of trastuzumab, pertuzumab, margetuximab, or 7C2.

Cell Surface Ligand or Receptor

In some embodiments, the cell killing agent is an antibody that specifically recognizes a ligand or receptor, such as extracellular domain of a ligand/receptor. In some embodiments, the ligand or receptor is derived from a molecule selected from the group consisting of NKG2A. NKG2C, NKG2F, NKG2D, BCMA, APRIL, BAFF, IL-3, IL-13, LLT1, AICL, DNAM-1, and NKp80. In some embodiments, the ligand is derived from APRIL and/or BAFF, which can bind to BCMA. In some embodiments, the receptor is an FcR and the ligand is an Fc-containing molecule. In some embodiments, the FcR is an Fcγ receptor (FcγR). In some embodiments, the FcγR is selected from the group consisting of FcγRIA (CD64A), FcγRIB (CD64B), FcγRIC (CD64C), FcγRIIA (CD32A), FcγRIIB (CD32B), FcγRIIIA (CD16a), and FcγRIIIB (CD16b).

Immune Cell Surface Antigen

In some embodiments, the cell killing agent is an antibody that specifically recognizes an immune cell surface antigen. Immune cells have different cell surface molecules. For example CD3 is a cell surface molecule on T-cells, whereas CD16, NKG2D, or NKp30 are cell surface molecules on NK cells, and CD3 or an invariant T-cell receptor (TCR) are the cell surface molecules on NKT-cells. In some embodiments, wherein the immune cell is a T-cell, the activation molecule is one or more of CD3, e.g., CD3ε, CD3δ, or CD3γ; or CD27, CD28, CD40, CD134, CD137, and CD278. In other some embodiments, wherein the immune cell is a NK cell, the cell surface molecule is CD16, NKG2D, or NKp30. In some embodiments, wherein the immune cell is a NKT-cell, the cell surface molecule is CD3 or an invariant TCR. In some embodiments, the immune cell is selected from the group consisting of a monocyte, a dendritic cell, a macrophage, a B cell, a killer T cell (T_{c}, cytotoxic T lymphocyte, or CTL), a helper T cell (Tₕ), a regulatory T cells (Treg), a γδ T cell, a natural killer T (NKT) cell, and a natural killer (NK) cell.

In some embodiments, the immune cell surface antigen is selected from the group consisting of CD3 (e.g., CD3ε, CD3δ, CD3γ), CD4, CD5, CD8, CD16, CD27, CD28, CD40, CD64, CD89, CD134, CD137, CD278, NKp46, NKp30, NKG2D, TCRα, TCRβ, TCRγ, and TCRδ. In some embodiments, the immune cell surface antigen is CD3, CD4, or CD8.

CD3 comprises three different polypeptide chains (ε, δ and γ chains), is an antigen expressed by T cells, including cytotoxic T cell (CD8+ naive T cells) and T helper cells (CD4+ naive T cells). The three CD3 polypeptide chains associate with the TCR and the ζ-chain to form the TCR complex, which has the function of activating signaling cascades in T cells. Currently, many therapeutic strategies target the TCR signal transduction to treat diseases using anti-human CD3 monoclonal antibodies. The CD3 specific antibody OKT3 is the first monoclonal antibody approved for human therapeutic use, and is clinically used as an immunomodulator for the treatment of allogenic transplant rejections. Otelixizumab (TRX4) is a monoclonal antibody specifically targeting CD3ε, and is being developed for the treatment of type I diabetes and other autoimmune diseases. In some embodiments, the antigen-binding domain or antibody specifically recognizing CD3 comprises heavy chain CDRs, light chain CDRs, or all six CDRs of OKT3 or otelixizumab. In some embodiments, the antigen-binding domain specifically recognizing CD3 comprises VH and/or VL of OKT3 or otelixizumab.

CD4 is a glycoprotein expressed on the surface of immune cells such as T helper cells (CD4+T helper cells), monocytes, macrophages, and dendritic cells. CD4 is a co-receptor of the TCR and assists TCR in communicating with antigen-presenting cells. Exemplary anti-CD4 antibodies include, but are not limited to, MAX.16H5 and IT1208. MAX.16H5 is an anti-human CD4 antibody applied intravenously in clinical trials for the treatment of autoimmune diseases (e.g., rheumatoid arthritis) and acute late-onset rejection after transplantation of a renal allograft. IT1208 is a defucosylated humanized anti-CD4 depleting antibody currently under clinical trial for treating advanced solid tumors. In some embodiments, the antigen-binding domain specifically recognizing CD4 comprises heavy chain CDRs, light chain CDRs, or all six CDRs of MAX.16H5 or IT1208. In some embodiments, the antigen-binding domain specifically recognizing CD4 comprises VH and/or VL of MAX.16H5 or IT1208.

CD8 is a transmembrane glycoprotein that serves as a co-receptor for TCR. CD8 binds to and is specific for MHC class I protein. The most common form of CD8 is composed of a CD8-α and CD8-β chain. CD8 is predominantly expressed on the surface of cytotoxic T cells, but can also be found on natural killer cells, cortical thymocytes, and dendritic cells. CD8 is a marker for cytotoxic T cell population. CD8 is expressed in T cell lymphoblastic lymphoma and hypo-pigmented mycosis fungoides.

In some embodiments, the cell-killing agent is an siRNA, a CRISPR/Cas, a ZFN, or a TALEN construct that targets the inhibitory immune checkpoint molecule described herein, to knockdown (KD) or knockout (KO) endogenous expression of such inhibitory checkpoint molecule in the target cell (e.g., tumor cell). In some embodiments, such cell-killing agent is introduced into the tumor cell together with the inducible reporter expressing construct. For example, a nucleic acid encoding such cell-killing agent (e.g., siRNA or CRISPR/Cas against PD-L1) and a nucleic acid encoding the reporter protein are on the same vector, either under the control of the same promoter, or under different promoter controls. In some embodiments, the nucleic acid encoding such cell-killing agent (e.g., siRNA or CRISPR/Cas against PD-L1) and the nucleic acid encoding the reporter protein are on different vectors, under control of the same or different promoters. The different vectors can be transduced into tumor cells simultaneously or sequentially, but before cell killing assay to obtain stable cell line. In some embodiments, the nucleic acid encoding such cell-killing agent (e.g., siRNA or CRISPR/Cas against PD-L1) is under control of a constitutive promoter (e.g., CMV). In some embodiments, the nucleic acid encoding such cell-killing agent (e.g., siRNA or CRISPR/Cas against PD-L1) is under control of an inducible promoter. By doing so, immunosuppression can be overcome or rescued to certain level (e.g., PD-L1 KO in inducible reporter tumor cells).

The cell-killing agent can be a combination of an immune cell and an immunomodulating agent (e.g., an antibody, an immune checkpoint inhibitor). Antibodies, such as checkpoint inhibitors, can function by modulating the immune system's endogenous mechanisms of T cell regulation. For example, Ipilimumab, an antibody that is an immune checkpoint inhibitor, binds and blocks inhibitory signaling mediated by the T cell (an immune cell) surface co-inhibitory molecule cytotoxic T lymphocyte antigen 4 (CTLA-4). In some embodiments, the cell-killing agent in the compositions or methods described herein is a combination of anti-HER2 antibody and an immune effector cell (e.g., NK, CTL, or PBMC). In some embodiments, the cell-killing agent in the compositions or methods described herein is a combination of anti-HER2/anti-CD3 antibody and an immune effector cell (e.g., NK, CTL, or PBMC). In some embodiments, the cell-killing agent in the compositions or methods described herein is a combination of anti-HER2/anti-CD47/anti-CD3 antibody or anti-PD-L1/anti-CD47/anti-CD3 antibody, and an immune effector cell (e.g., NK, CTL, or PBMC). In some embodiments, the cell-killing agent in the compositions or methods described herein is a combination of 1) an anti-PD-1 antibody or an anti-PD-L1 antibody. 2) an anti-HER2/anti-CD3 antibody, an anti-HER2/anti-CD47/anti-CD3 antibody, or an anti-PD-L1/anti-CD47/anti-CD3 antibody, and 3) an immune effector cell (e.g., NK, CTL, or PBMC).

The immunomodulating agent and the immune cell can be pre-incubated to form the cell-killing agent, and then contacted to the tumor cells of the disclosure. In some embodiments, the immunomodulating agent and the immune cell are not pre-incubated to form a cell-killing agent. Instead, the immunomodulating agent and the immune cell can be added sequentially or simultaneously to the tumor cells, whereupon the immunomodulating agent and the immune cell can bind together to form the cell-killing agent. The immunomodulating agent can be added to the tumor cells before the immune cell is added. The immunomodulating agent can be added to the tumor cells after the immune cell is added to the tumor cells. The immunomodulating agent can be added at the same time as the immune cell to the tumor cells. When the immunomodulating agent binds to the immune cell (i.e., a protein or receptor expressed on the immune cell) the reaction can form a cell-killing agent.

### Media

Tumor cells can be grown in any suitable medium that supports the growth of the tumor cells. Culture medium compositions can include essential amino acids, salts, vitamins, minerals, trace metals, sugars, lipids and nucleosides. Cell culture medium attempts to supply the components necessary to meet the nutritional needs required to grow cells in a controlled, artificial and in vitro environment. Nutrient formulations, pH, and osmolarity vary in accordance with parameters such as cell type, cell density, and the culture system employed. Many cell culture medium formulations are documented in the literature and a number of media are commercially available.

Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the tumor cells, or any cells of the disclosure. Any of media may be supplemented with hormones and/or other growth factors (such as insulin, transferrin, albumin, or epidermal growth factor), salts (such as sodium chloride, calcium, magnesium, and phosphate), buffers (such as HEPES), nucleotides (such as adenosine and thymidine), amino acids (e.g., L-glutamine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the tumor cell selected for expression, and will be apparent to the ordinarily skilled artisan.

Once the culture medium is incubated with cells, it is known to those skilled in the art as “spent” or “conditioned medium.” Conditioned medium contains many of the original components of the medium, as well as a variety of cellular metabolites and secreted proteins, including, for example, biologically active growth factors, inflammatory mediators and other extracellular proteins. In some instances, the conditioned medium comprises a secretable reporter protein of the disclosure.

In some embodiments, there is provided a composition comprising a tumor cell (e.g., inducible reporter tumor cell), a non-tumor cell (e.g., fibroblast), a cell-killing agent (e.g., small compound, immune effector cell, antibody such as multispecific antibody, ADC, immunomodulator such as immune checkpoint inhibitor, etc., or any combinations thereof), a nucleic acid encoding a reporter protein (e.g., under inducible promoter control), an induction agent (e.g., doxycycline), and a secreted reporter protein in media (e.g., luciferase or GFP), or any combination thereof. For example, in some embodiments, there is provided a composition comprising a tumor cell of the disclosure comprising a nucleic acid encoding a reporter protein (e.g., under inducible promoter), and secreted reporter protein (e.g., luciferase or GFP) in media in which the tumor cell is growing. In some embodiments, there is provided a composition comprising a tumor cell comprising a nucleic acid encoding a reporter protein (e.g., under inducible promoter), a secreted reporter protein (e.g., luciferase or GFP), and a cell-killing agent (e.g., small compound, immune effector cell, antibody such as multispecific antibody, ADC, immunomodulator such as immune checkpoint inhibitor, etc., or any combinations thereof) in media in which the tumor cell is growing. In some embodiments, the composition further comprises a second reporter protein secreted by the tumor cells. In some embodiments, the composition further comprises an induction agent (e.g., doxycycline).

### Kits

The disclosure provides for kits useful for practicing the methods of the disclosure. A kit can include any of the components described herein, including but not limited to, a tumor cell, a non-tumor cell (e.g., fibroblast), a cell-killing agent (or combination of cell-killing agents), and induction agent, a nucleic acid encoding a reporter protein operably linked to an inducible promoter, or any combination thereof. In some embodiments, the kit further comprises a second nucleic acid encoding a second reporter protein operably linked to a second inducible promoter. In some embodiments, the kit further comprises a third nucleic acid encoding a KO construct (e.g., siRNA, CRISPR/Cas, ZFN, or TALEN), such as for targeting an endogenous inhibitory checkpoint molecule (e.g., PD-L1).

The kit can also comprise any reagents described herein and/or useful for practicing the methods of the disclosure. Reagents can include reagents for growing cells, reagents for incorporating a nucleic acid encoding a reporter protein into a cell, reagents for diluting components of the kit, and reagents for solubilizing components of the kit. Reagents can include buffers. Suitable buffering agents for use in the present application can include both organic and inorganic acids and salts thereof. For example, citrate, phosphate, succinate, tartrate, fumarate, gluconate, oxalate, lactate, acetate. Buffers may comprise histidine and trimethylamine salts such as Tris.

The kits of the disclosure can in suitable packaging. Suitable packaging can include, but is not limited to, vials, bottles, jars, flexible packaging (e.g., sealed Mylar or plastic bags), and the like. Components of the kits may be present in separate containers, or multiple components may be present in a single container. For example, the tumor cell and non-tumor cell may be provided in separate containers, or may be provided in a single container.

In addition to above-mentioned components, the kit may further include instructions for using the components of the kit to practice the methods of the disclosure. The instructions for practicing the method can be recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. The instructions can be present as an electronic storage data file present on a suitable computer readable storage medium, e.g. CD-ROM, diskette, Hard Disk Drive (HDD) etc. The actual instructions may not present in the kit, but means for obtaining the instructions from a remote source, e.g. via the internet (i.e., through storage in the cloud), are provided. An example of this embodiment is a kit that includes a web address where the instructions can be viewed and/or from which the instructions can be downloaded. As with the instructions, this means for obtaining the instructions is recorded on a suitable substrate.

### Exemplary Embodiments

Embodiment 1. A method of evaluating the effectiveness of a cell-killing agent on a population of tumor cells, the method comprising: a. contacting the tumor cells with a cell-killing agent, wherein each of the tumor cells comprises a nucleic acid encoding a reporter protein, wherein expression of the nucleic acid is controlled by an inducible promoter; b, inducing expression of the nucleic acid to produce the reporter protein: and c. determining the amount of the reporter protein, wherein the amount of the reporter protein negatively correlates with the effectiveness of the cell killing agent.

Embodiment 2. The method of embodiment 1, wherein the contacting step occurs before the inducing step.

Embodiment 3. The method of embodiment 1, wherein the contacting step occurs simultaneously with the inducing step.

Embodiment 4. The method of embodiment 1 or 2, wherein the contacting step occurs for at least about 24 hours prior to the inducing step.

Embodiment 5. The method of embodiment 4, wherein the contacting step occurs for about 4 to about 48 hours prior to the inducing step.

Embodiment 6. The method of embodiment 1, wherein the contacting step occurs for up to about 6 days prior to the inducing step.

Embodiment 7. The method of any one of embodiments 1-6, wherein the inducing step occurs for about 4-8 hours.

Embodiment 8. The method of any one of embodiments 1-7, wherein the inducing step comprises treating the tumor cells with an induction agent.

Embodiment 9. The method of embodiment 8, wherein the induction agent is selected from the group consisting of: tetracycline, doxycycline, estrogen receptor, and cumate, or any combination thereof.

Embodiment 10. The method ofany one of embodiments 1-9, wherein the reporter protein is secreted by the tumor cells.

Embodiment 11. The method of embodiment 10, wherein the reporter protein is luciferase.

Embodiment 12. The method of embodiment 10, wherein the reporter protein is selected from the group consisting of: Oplophorus luciferase, beetle luciferase, Renilla luciferase, Metridia luciferase, Gaussia luciferase, secreted alkaline phosphatase, secreted fluorescent protein, and NANOLUC luciferase, or any combination thereof.

Embodiment 13. The method of any one of embodiments 1-12, wherein the determining step comprises detecting the reporter protein over different time points.

Embodiment 14. The method of any one of embodiments 1-13, wherein the tumor cells are present in a mixture comprising a second population of cells.

Embodiment 15. The method of embodiment 14, wherein the second population of cells are selected from the group consisting of fibroblast cells, stromal cells, endothelial cells, tumor associated macrophages, myeloid-derived suppressive cells, or any combination/variant thereof, or any combination thereof.

Embodiment 16. The method of any one of embodiments 1-15, wherein the tumor cells are present in a 3D spheroid or a 2D monolayer.

Embodiment 17. The method of any one of embodiments 1-16, wherein the cell-killing agent is selected from the group consisting of: a cytotoxin, a drug, a small molecule, and a small molecule compound, or any combination thereof.

Embodiment 18. The method of any one of embodiments 1-16, wherein the cell-killing agent is an immune cell.

Embodiment 19. The method of embodiment 18, wherein the immune cell is selected from the group consisting of an NK cell, an NKT cell, a T cell, a CAR-T cell, a CD14+ cell, a dendritic cell, and a PBMC cell, or any combination thereof.

Embodiment 20. The method of any one of embodiments 1-16, wherein the cell-killing agent is an immunomodulating agent, and wherein the contacting step is conducted in the presence of immune cells.

Embodiment 21. The method of embodiment 20, wherein the immunomodulating agent is an immune checkpoint inhibitor.

Embodiment 22. The method of embodiment 21, wherein the immune checkpoint inhibitor is selected from the group consisting of PD-1, PD-L1, PD-L2, Siglec, BTLA, CTLA-4, and CD20, or any combination thereof.

Embodiment 23. The method of any one of embodiments 1-16, wherein the cell-killing agent is an antibody.

Embodiment 24. The method of embodiment 24, wherein the antibody is selected from the group consisting of anti-PD-1, anti-PD-L1, anti-CD47, anti-HER2, Herceptin, anti-CD20, and anti-CD3 antibodies, or any combination thereof.

Embodiment 25. The method of any one of embodiments 1-24, wherein the nucleic acid is introduced into the cells by a retroviral or lentiviral vector system.

Embodiment 26. The method of embodiment 1, wherein the tumor cells further comprise a second nucleic acid encoding a second reporter protein.

Embodiment 27. The method of embodiment 26, wherein the expression of the second nucleic acid is controlled by an inducible promoter.

Embodiment 28. The method of embodiment 27, wherein the second reporter protein is GFP.

Embodiment 29. A composition comprising: a population of tumor cells, wherein each of the tumor cells comprise a nucleic acid encoding a reporter protein, wherein expression of the nucleic acid is controlled by an inducible promoter.

Embodiment 30. The composition of embodiment 29, wherein reporter protein is secreted by the tumor cells.

Embodiment 31. The composition of embodiment 29 or 30, wherein the reporting protein is luciferase.

Embodiment 32. The composition of embodiment 31, wherein the luciferase is a luciferase selected from the group consisting of: Oplophorus luciferase, beetle luciferase, Renilla luciferase, Metridia luciferase, Gaussia luciferase, secreted alkaline phosphatase, secreted fluorescent protein, and NANOLUC luciferase, or any combination thereof.

Embodiment 33. The composition of any one of embodiments 29-32, wherein composition further comprises a second population of cells.

Embodiment 34. The composition of embodiment 33, wherein the second population of cells are selected from the group consisting of fibroblast cells, stromal cells, endothelial cells, tumor associated macrophages, myeloid-derived suppressive cells, or any combination/variant thereof, or any combination thereof.

Embodiment 35. The composition of any one of embodiments 29-34, wherein the composition is a 3D spheroid or a 2D monolayer.

Embodiment 36. The composition of any one of embodiments 29-35, further comprising a cell killing agent.

Embodiment 37. The method of embodiment 36, wherein the cell-killing agent is selected from the group consisting of: a cytotoxin, a drug, a small molecule, and a small molecule compound, or any combination thereof.

Embodiment 38. The composition of embodiment 36, wherein the cell-killing agent is an immune cell.

Embodiment 39. The composition of embodiment 38, wherein the immune cell is selected from the group consisting of an NK cell, an NKT cell, a T cell, a CAR-T cell, a CD14+ cell, a dendritic cell, and a PBMC cell, or any combination thereof.

Embodiment 40. The composition of embodiment 36, wherein the cell-killing agent is an immunomodulating agent, and wherein the contacting step is conducted in the presence of immune cells.

Embodiment 41. The composition of embodiment 40, wherein the immunomodulating agent is an immune checkpoint inhibitor.

Embodiment 42. The composition of embodiment 41, wherein the immune checkpoint inhibitor is selected from the group consisting of PD-1, PD-L1, PD-L2, Siglec, BTLA, CTLA-4, and CD20, or any combination thereof.

Embodiment 43. The composition of embodiment 36, wherein the cell-killing agent is an antibody.

Embodiment 44. The composition of embodiment 43, wherein the antibody is selected from the group consisting of anti-PD-1, anti-PD-L1, anti-CD47, anti-HER2, Herceptin, anti-CD20, and anti-CD3 antibodies, or any combination thereof.

Embodiment 45. The composition of any one of embodiments 29-44, further comprising an induction agent selected from the group consisting of: tetracycline, doxycycline, estrogen receptor, and cumate, or any combination thereof.

Embodiment 46. The composition of any one of embodiments 29-45, further comprising a reporter protein secreted by the tumor cells.

Embodiment 47. The composition of embodiment 46, wherein the reporting protein is luciferase.

Embodiment 48. The composition of embodiment 47, wherein the luciferase is a luciferase selected from the group consisting of: Oplophorus luciferase, beetle luciferase. Renilla luciferase, Metridia luciferase, Gaussia luciferase, secreted alkaline phosphatase, secreted fluorescent protein, and NANOLUC luciferase, or any combination thereof.

Embodiment 49. The composition of any one of embodiments 2948, wherein the tumor cells further comprises a second nucleic acid encoding a second reporter protein.

Embodiment 50. The composition of embodiment 49, wherein the second reporter protein comprises an intracellular fluorescent protein.

Embodiment 51. The composition of embodiment 50, wherein the second protein is GFP.

Embodiment 52. The composition of any one of embodiments 29-51, wherein the composition comprises an immunomodulating agent, and wherein the composition further comprise an immune cell.

### EXAMPLES

The following examples are offered by way of illustration and not by way of limitation.

### Example 1: Molecular Construct for the Expression of Dual Reporters snLuciferase and GFP

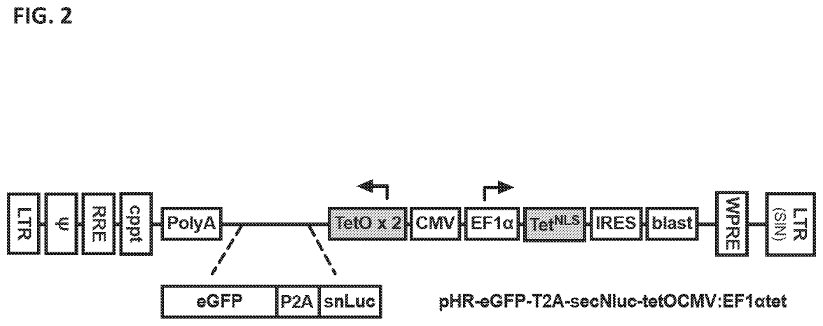

This example shows an exemplary molecular construct “pHR-eGFP-T2A-secNluc-tetOCMV:EF1αtet” for the expression of dual reporter proteins (i.e., luciferase and GFP) under a Tet-on system (hereinafter referred to as “Tet-on snLuc-GFP construct”). As shown in FIG. 2, in the absence of an induction agent, such as doxycycline, the Tet repressor is constitutively expressed under an EF1α promotor and represses transcription of the reporter genes under the CMV promotor. The addition of doxycycline (“dox”) activates transcription of the dual markers, GFP and snLuciferase. This construct was cloned into a self-inactivating (SIN) lentiviral vector, and transduced into various tumor cell lines (hereinafter referred to as “dual reporter tumor cells”) for later experiments.

### Example 2: GFP Signal Generated in Tumor Cells is Proportional to the Number of Live Tumor Cells Under Co-Culture Conditions with Immune Effector Cells (Nonspecific Killing)

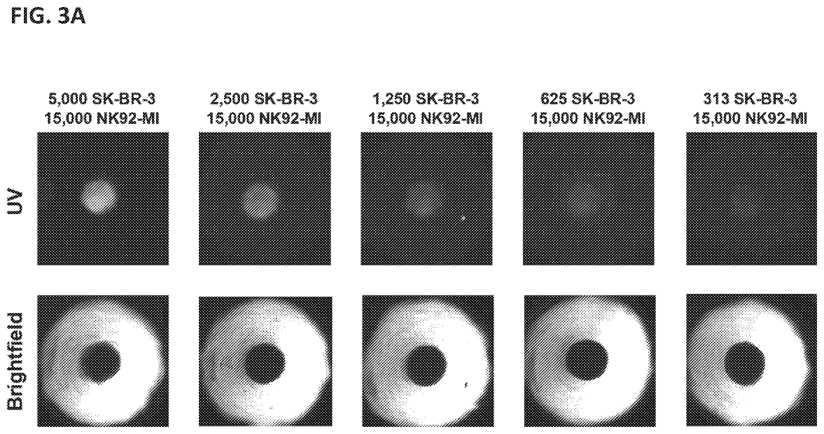

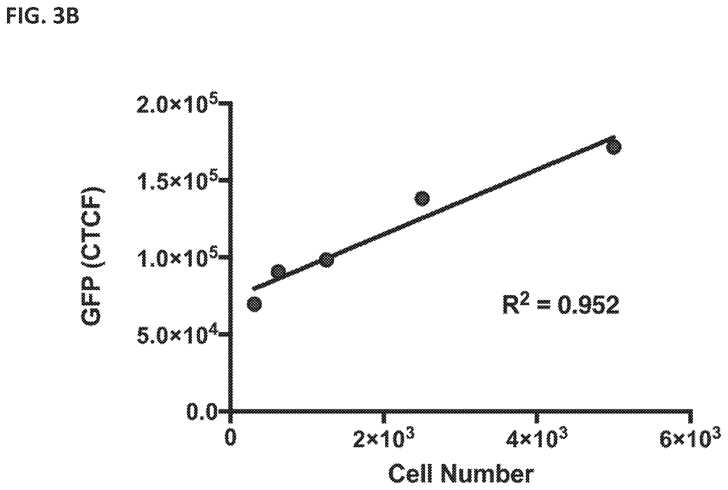

This example shows that the GFP signal generated from dual reporter tumor cells in a sample is proportional to the number of live tumor cells in that sample. SK-BR-3 cells (human breast cancer cell line) were transduced with the Tet-on snLuc-GFP construct in Example 1 to express GFP and snLuciferase under a dox-inducible system (hereinafter referred to as “dual reporter SK-BR-3 cells”). NK92-MI (ATCC® CRL-2408) cells (Natural Killer Cell line) were plated in a 96-well plate at 15,000 cells/well in the presence of different concentrations of dual reporter SK-BR-3 cells (serially diluted 2-fold, ranging from about 5000 cells to about 313 cells per well). No cell killing agent was added. NK cells can perform nonspecific killing via killer-cell immunoglobulin-like receptor (KIR) recognition of MHC on tumor cells. Dual reporter SK-BR-3 cells were analyzed 24 hours after doxycycline induction using a Nikon Ellipse TE2000-U microscope. The GFP signal intensity was quantified using the corrected total cell fluorescence (CTCF) measurements obtained from ImageJ. GFP intensity was found to linearly correlate (R²=0.952) with the number of alive dual reporter SK-BR-3 cells, as shown in FIGS. 3A-3B. These results demonstrate that GFP may serve as a semi-quantitative marker to monitor live tumor cells in the methods of the disclosure.

### Example 3: snLuciferase can Serve as a Semi-Quantitative Marker for Live Tumor Cells

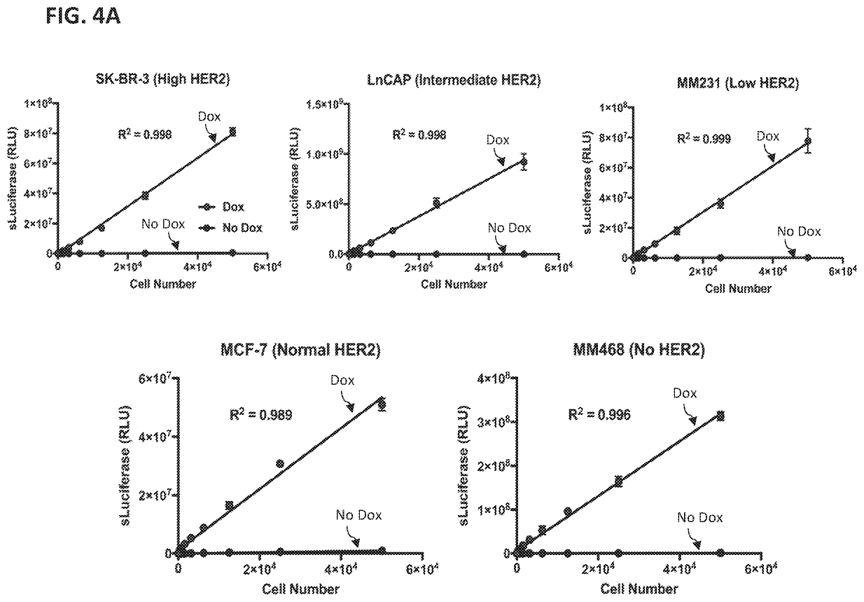

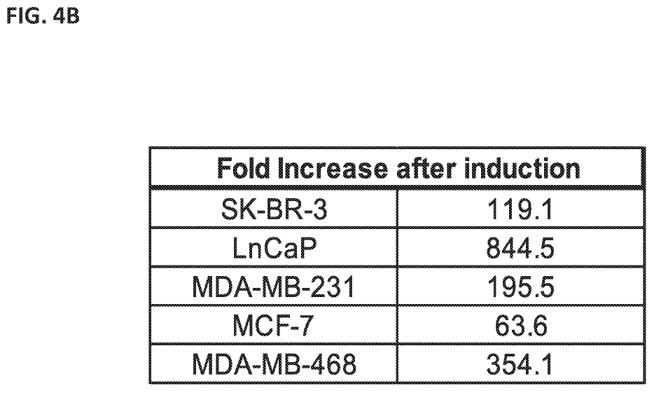

This example shows that snLuciferase signal generated from dual reporter tumor cells in a sample is correlated with the number of live tumor cells in that sample. Five target tumor cell lines expressing variable amounts of HER2 antigen (human breast cancer cell line SK-BR-3 with high HER2 expression, human prostate adenocarcinoma cell line LnCAP with intermediate HER2 expression, human triple-negative breast cancer cell line MDA-MB-231 with low HER2 expression, human breast cancer cell line MCF-7 with normal HER2 expression similar to healthy tissue cells, and human breast cancer cell line MDA-MB-468 with no HER2 expression) were transduced with the Tet-on snLuc-GFP construct in Example 1 to express GFP and snLuciferase under a dox-inducible system. The transduced tumor cells were plated at different concentrations in a 96-well conical bottom plate and serially diluted 2-fold from about 50,000 to about 50 cells per well. snLuciferase was measured 24 hours after doxycycline induction using a GloMax Discover Microplate Reader. These dual reporter tumor cells were also analyzed under non-doxed conditions to determine the basal expression level of snLuciferase (i.e., leakiness). Linear regression graphs were generated from four replicate measurements as shown in FIG. 4A. A linear relationship between snLuciferase luminescence and the number of live tumor cells was observed, suggesting that snLuciferase can serve as a semi-quantitative marker to monitor live tumor cells in the methods of the disclosure.

FIG. 4B shows that induction can increase expression of snLuciferase by about 50- to about 850-fold. High basal expression levels of the snLuciferase reporter can negatively affect the sensitivity of detection, particularly with methods that require a long incubation time (i.e., multiple days). Tight control of the expression of the reporter proteins can contribute to reducing background noise in the methods of the disclosure.

### Example 4: Measuring Antibody Dependent Cell-Mediated Cytotoxicity (ADCC) Using snLuciferase

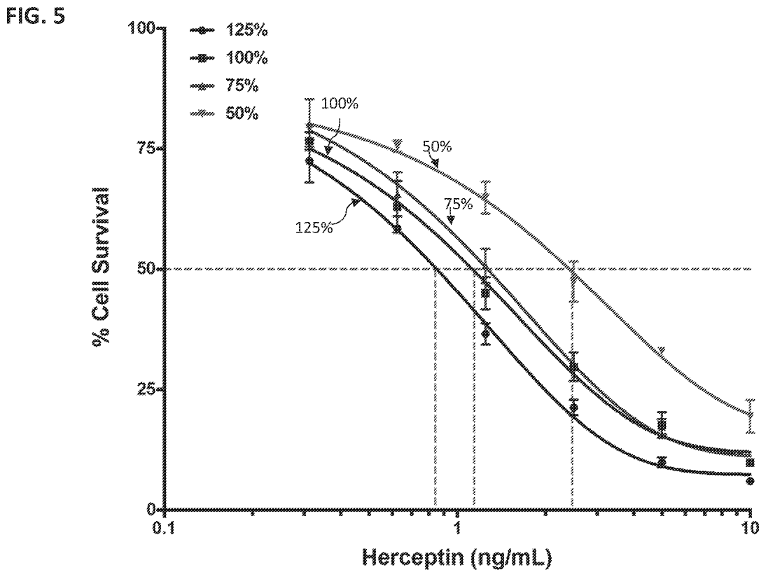

This example shows that the methods of the disclosure can be used to assay for antibody dependent cell-mediated cytotoxicity (ADCC) based on snLuciferase measurement. 15,000 NK92 cells (effector cells) and 5,000 dual reporter SK-BR-3 cells (breast cancer tumor cells) as constructed in Example 2 were plated in a 96-well conical bottom plate, resulting in a 3:1 effector cell to tumor cell ratio. Different concentrations of Herceptin® (anti-HER2 antibody) serially diluted 2-fold were added into experimental wells (100% relative potency was defined as 10 ng/mL-0.31 ng/mL of antibody). Control wells were not provided with Herceptin®. The cell-killing reaction was incubated for 8 hours. After the 8 hours incubation, doxycycline was added to the cell mixture to induce expression of the dual reporters from remaining dual reporter SK-BR-3 cells. 24 hours after doxycycline induction, snLuciferase was measured using a GloMax Discover Microplate Reader. Percent cell survival was calculated as the ratio of relative light unit (RLU) in Herceptin®-containing wells to the RLU in the Herceptin®-free control wells. The dose-response curves were generated from four replicates, as shown in FIG. 5. ADCC activity was distinguished at 50%, 75%, 100%, and 125% relative potency. The potencies are relative to the different concentrations of antibody (i.e., the highest dose tested at 125% potency is 12.5 ng, the highest dose tested at 100% potency is 10 ng, the highest dose tested at 75% potency is 7.5 ng, the highest dose tested at 50% potency is 5 ng). As shown in FIG. 5, Herceptin® shows a concentration-dependent ADCC on dual reporter SK-BR-3 cells when co-incubated with effector cells NK92. These results demonstrate that snLuciferase and accompanying methods of the disclosure can serve as a sensitive, semi-quantitative marker to monitor ADCC activity.

### Example 5: Measuring Antibody Dependent Cell-Mediated Cytotoxicity (ADCC) Using GFP

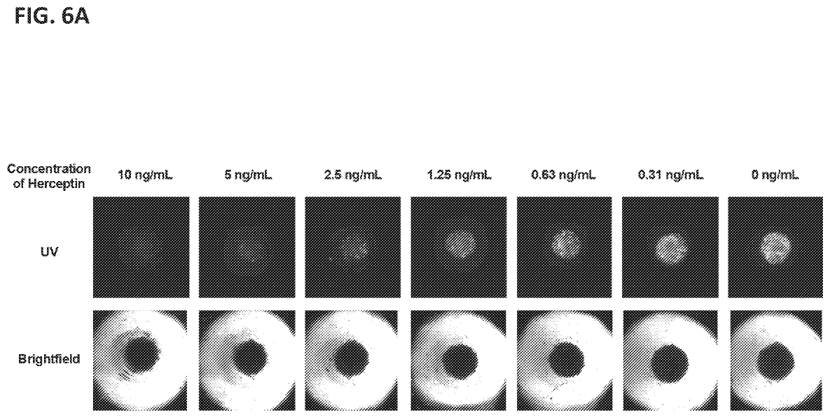

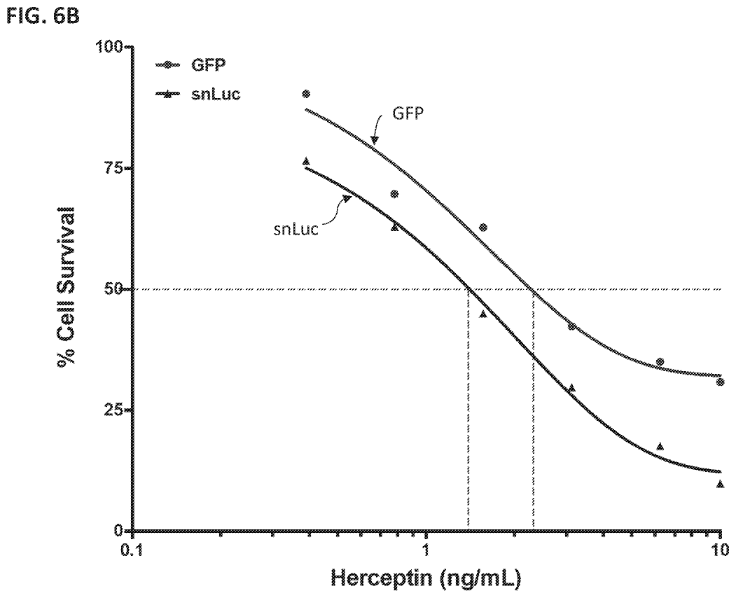

This example shows that the methods of the disclosure can be used to assay for ADCC based on GFP measurement. 15,000 NK92 cells (effector cells) and 5,000 dual reporter SK-BR-3 cells (tumor cells) as constructed in Example 2 were plated in a 96-well conical bottom plate, resulting in a 3:1 effector cell to tumor cell ratio. Different concentrations of Herceptin® serially diluted 2-fold, ranging from 10 ng/mL-0.31 ng/mL, were added into experimental wells. Control wells were not provided with Herceptin® (0 ng/mL). The cell killing reaction was incubated for 8 hours. After 8-hour incubation, doxycycline was added to the mixture of cells to induce expression of the dual reporters from remaining dual reporter SK-BR-3 cells. 24 hours after doxycycline induction, the tumor cells were analyzed using a Nikon Ellipse TE2000-U microscope for GFP, and snLuciferase was measured using a GloMax Discover Microplate Reader. The GFP signal was quantified using ImageJ. Percent cell survival was calculated as the ratio of corrected total cell fluorescence (CTCF) in Herceptin®-containing wells to the CTCF in the Herceptin®-free control wells. A dose-dependent relationship between GFP signal and antibody concentration was observed, as shown in FIGS. 6A-6B. These results demonstrate that GFP may serve as a marker to visualize and monitor ADCC.

For snLuciferase measurement, percent cell survival was calculated as the ratio of relative light unit (RLU) in Herceptin®-containing wells to the RLU in the Herceptin®-free control wells. As shown in FIG. 6B, the dose-response curve generated from snLuciferase measurement shifted left from the curve generated from GFP measurement, which may be explained by difficulties in obtaining quantitative fluorescent microscopy measurements. Therefore, snLuciferase may serve as a more sensitive semi-quantitative marker than GFP.

### Example 6: Measuring Immune Effector-Cell Killing Mediated by Immunotherapy Using snLuciferase

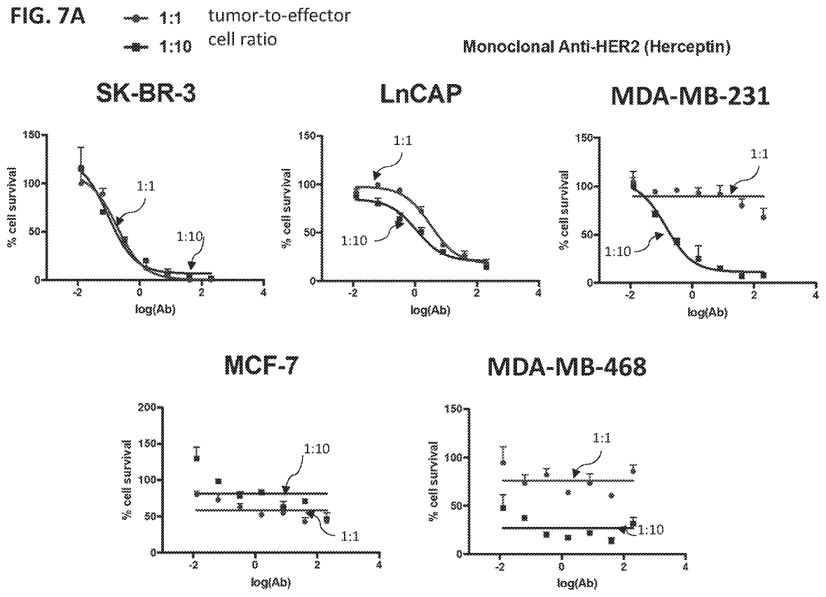

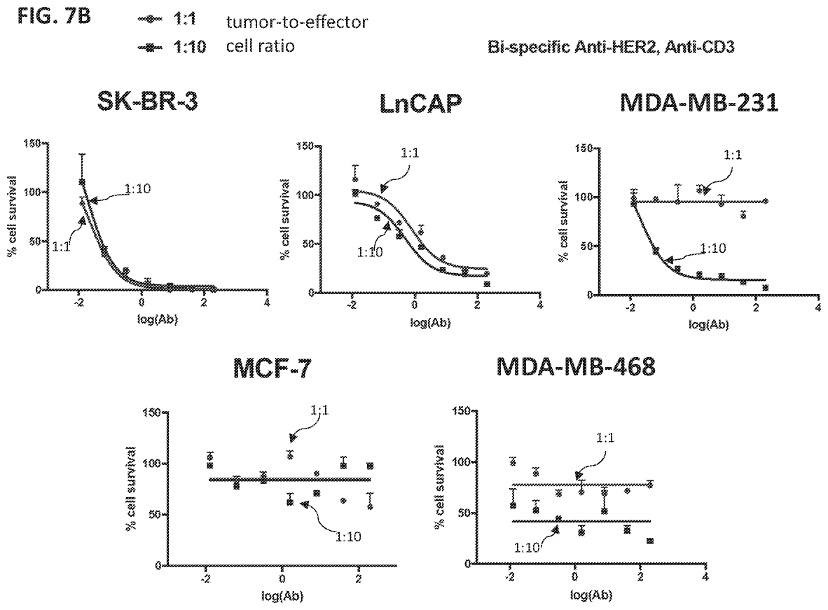

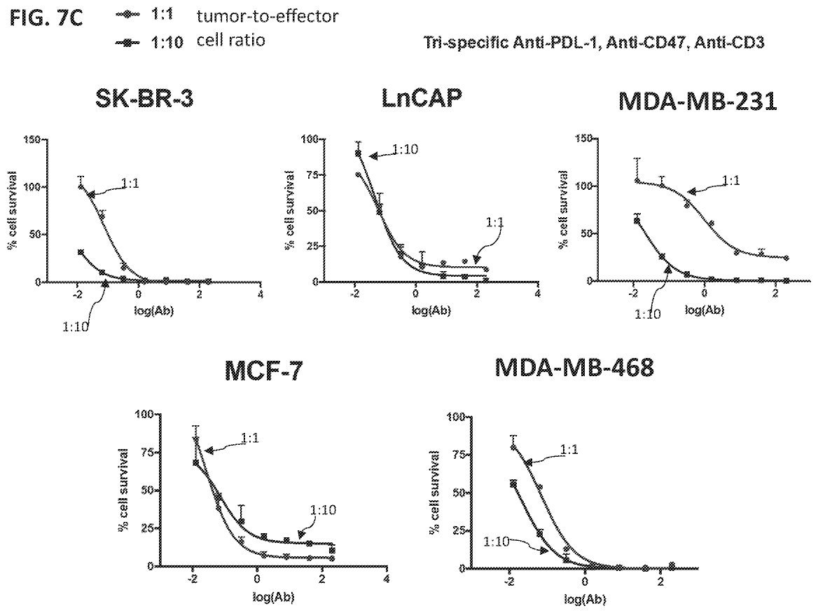

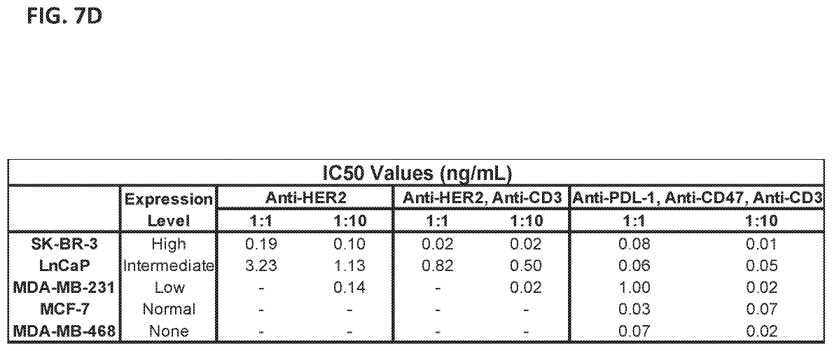

This example shows that cell killing by immunotherapeutic agents is dependent on the type of cell-killing agent, the dose of the cell-killing agent, the antigen expression level of the tumor cell (cell type), and the effector to target ratio used in the assay. Dual reporter tumor cell lines SK-BR-3 (high HER2 expression), LnCaP (intermediate HER2 expression), MDA-MB-231 (low HER2 expression), MCF-7 (normal HER2 expression similar to healthy tissue cells), and MDA-MB-468 (no HER2 expression) as constructed in Example 3 expressing various amounts of HER2 antigen were plated into wells of a %-well conical bottom plate at 5,000 tumor cells/well in the presence of 5,000 or 50,000 unstimulated PBMCs (effector cells), resulting in a 1:1 or 1:10 ratio of tumor cells to effector cells. Various antibodies (monoclonal anti-HER2 antibody Herceptin®, bi-specific anti-HER2/anti-CD3 antibody, and tri-specific anti-PD-L1/anti-CD47/anti-CD3 antibody) were added to the mixture of cells at different concentrations, 5-fold serial dilution, ranging from 200 ng/mL to 0.0128 ng/mL. Control wells were not provided with antibodies. The cell-killing reaction was incubated for 48 hours. After 48-hour incubation, doxycycline was added to the mixture of cells to induce expression of the dual reporters from live dual reporter tumor cells. 24 hours after doxycycline induction, cell culture media was taken, and luminescence was monitored on a GloMax Discover Microplate Reader. The dose-response curves were generated from two replicate measurements. As shown in FIGS. 7A-7D, cell killing responses based on the methods of the disclosure is dependent on type of antibody used, dose of the antibody, antigen expression level of the tumor cells, and the effector to target ratio.

FIG. 7A shows that the methods of the disclosure can detect antigen-dependent (HER2) and dose-dependent killing through CD16 (Fc receptor. “FcR”) positive cells (i.e., NK and NKT cells) in unstimulated PBMCs via Herccptin® mediated ADCC. As shown in FIGS. 7A and 7D, Herceptin® mediated ADCC of effector cells on tumor cells expressing high HER2 level is stronger than those expressing low HER2 level (compare SK-BR-3, LnCAP, and MDA-MB-231); the smaller the tumor-to-effector cell ratio (i.e., more effector cells) the stronger the ADCC effect (e.g., compare 1:10 with 1:1 in MDA-MB-231 panel). Herceptin® did not mediate antibody concentration-dependent ADCC of effector cells on tumor cells with normal HER2 expression as in healthy tissue cells (MCF-7), or on tumor cells not expressing HER2 (MDA-MB-468). The different cell survival rates under tumor-to-effector cell ratio of 1:10 and 1:1 in MCF-7 and MDA-MB-468 cells were likely due to Herceptin®, independent cell killing by PBMCs (e.g., NK cell non-specific killing).

The bispecific anti-HER2/anti-CD3 antibody (made in house) used in this experiment has no Fc function due to LALA mutations and cannot mediate ADCC. FIG. 7B shows that the methods of the disclosure can detect antigen-dependent (HER2) and dose-dependent cell-killing through CD3 positive cells (i.e., T-cells) in unstimulated PBMCs. As shown in FIGS. 7B and 7D, bispecific anti-HER2/anti-CD3 antibody targeted CD3+ effector cells to HER2+ tumor cells for cell-killing, with stronger cell-killing effect on tumor cells expressing high HER2 level than those expressing low HER2 level (compare SK-BR-3, LnCAP, and MDA-MB-231): the smaller the tumor-to-effector cell ratio (i.e., more effector cells) the stronger the cell-killing effect (e.g., compare 1:10 with 1:1 in MDA-MB-231 panel). Bispecific anti-HER2/anti-CD3 antibody did not mediate antibody concentration-dependent effector cell-killing on tumor cells with normal HER2 expression as in healthy tissue cells (MCF-7), or on tumor cells not expressing HER2 (MDA-MB-468). The different cell survival rates under tumor-to-effector cell ratio of 1:10 and 1:1 in MDA-MB468 cells were likely due to antibody independent non-specific cell killing by PBMCs (e.g., NK cell)—the higher E:T ratio, the higher non-specific cell killing.

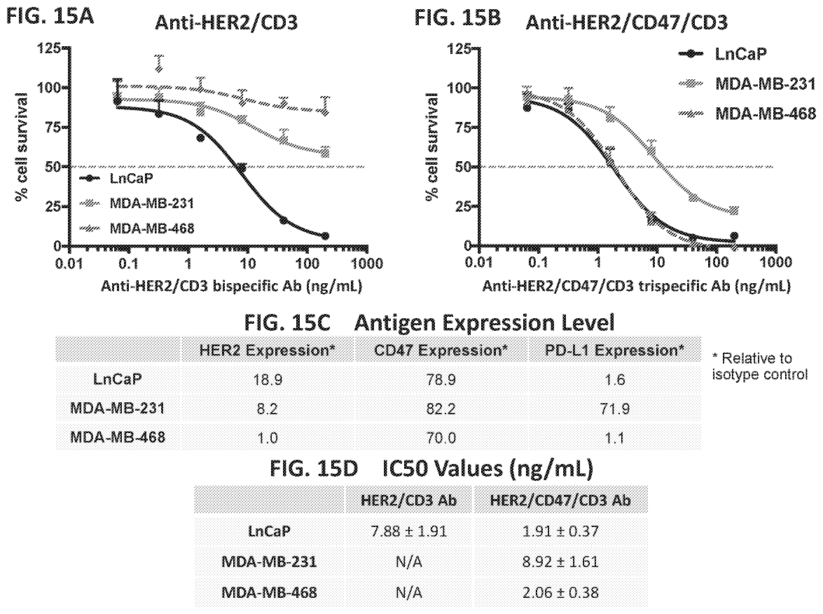

The trispecific anti-PD-L1/anti-CD47/anti-CD3 antibody (made in house) used in this experiment has no Fc function due to LALA mutations and cannot mediate ADCC. All tumor cell lines express CD47 (relatively high in SK-BR-3, MDA-MB-231, LnCaP, MCF-7, and MDA-MB-468 cells) and/or PD-L1 (high in MDA-MB-231 and low/no in SK-BR-3, LnCaP, MCF-7, and MDA-MB-468). Also see FIG. 15C. FIG. 7C shows that the methods of the disclosure can detect antigen-dependent (PD-L1 or CD47) and dose-dependent killing through CD3 positive cells (i.e., T-cells) in unstimulated PBMCs. As shown in FIGS. 7C-7D, for almost all tumor cells tested, the smaller the tumor-to-effector cell ratio (i.e., more effector cells) the stronger the cell-killing effect. However, higher nonspecific killing (e.g., from NK cells) was associated with smaller tumor-to-effector cell ratio.

### Example 7: Measuring the Kinetics of T-Cell Killing Mediated by Immunotherapy by Monitoring snLuciferase

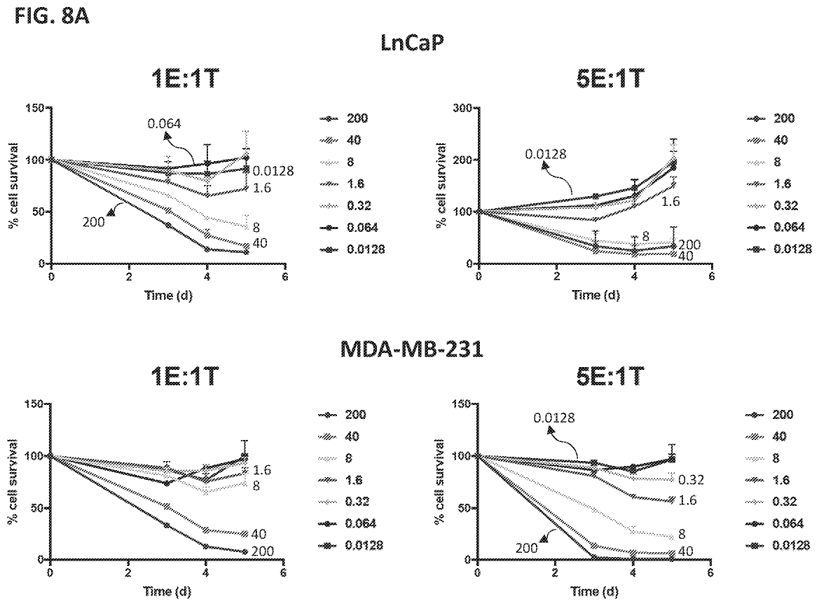

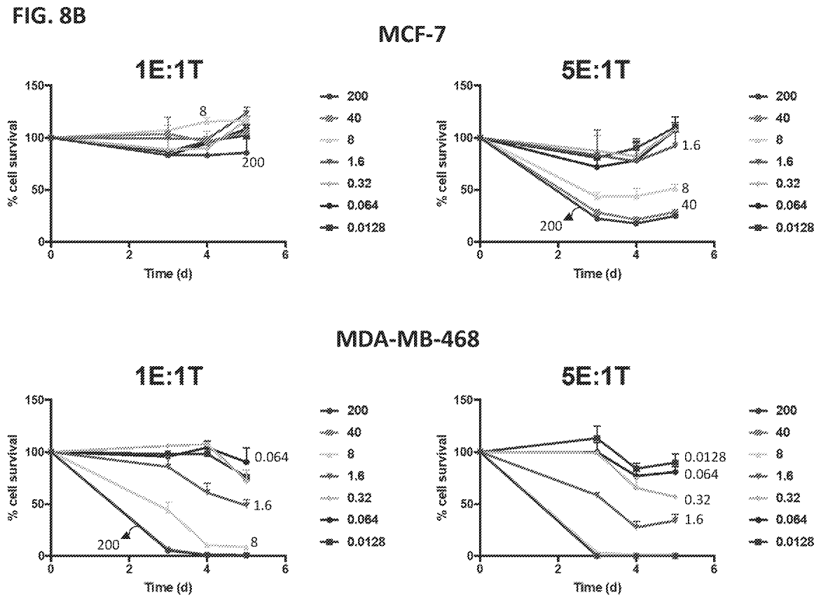

This example shows that the methods of the disclosure allow for assaying immunotherapy-mediated effector cell killing of tumor cells over time. Dual reporter cells LnCaP, MDA-MB-231, MCF-7, and MDA-MB-468 as constructed in Example 3 expressing various amounts of HER2 antigen were plated into wells of a 96-well conical bottom plate at 30,000 tumor cells/well in the presence of 30,000 or 150,000 unstimulated PBMCs (effector cells), resulting in a 1:1 or 5:1 ratio of effector to target cell ratio. A trispecific anti-HER2/anti-CD47/anti-CD3 antibody (made in house) was added to the cells at different concentrations, with 5-fold serial dilutions, from 200 ng/mL to 0.0128 ng/mL. Control wells were not provided with antibodies. The cell-killing reaction was incubated for 48 hours. After the 48-hour incubation, doxycycline was added to the mixture of cells to induce expression of the dual reporters from live dual reporter tumor cells. Luminescence was monitored on a GloMax Discover Microplate Reader at different time points post-induction. The time courses were generated from two replicate measurements. As shown in FIGS. 8A-8B, cell killing activity (e.g., reflected by cell viability) based on the methods of the disclosure can be measured continuously and in real time.

The trispecific anti-HER2/anti-CD47/anti-CD3 antibody has no Fc function due to LALA mutations and cannot mediate ADCC. The inclusion of a CD47 antigen binding domain allows the trispecific antibody to bypass HER2-dependent killing (e.g., compare antibody-mediated effector cell killing on MCF-7 cells (normal HER2 expression as in healthy tissue cells, high CD47 expression) and MDA-MB-468 cells (no HER2 expression, high CD47 expression) in FIG. 8B and FIG. 7B). The inclusion of a CD47 antigen binding domain also allows the trispecific antibody to act synergistically with the HER2 antigen binding domain to effect effector-mediated tumor cell killing (e.g., compare cytotoxicity of 1:1 E:T ratio on MDA-MB-231 (low HER2 expression) cells in FIG. 8A and FIG. 7B). FIGS. 8A-8B show that the methods of the disclosure can detect antigen-dependent (HER2 and/or CD47) and antibody dose-dependent effector cell killing through CD3 positive cells (i.e., T-cells). As can be seen from FIGS. 8A-8B, higher antibody concentration and/or higher effector-to-tumor cell ratio can result in stronger antibody-mediated effector cell killing on tumor cells.

### Example 8: Measuring Antibody Mediated T-Cell Killing of Tumor Cells in 3D Fibroblast Spheroids

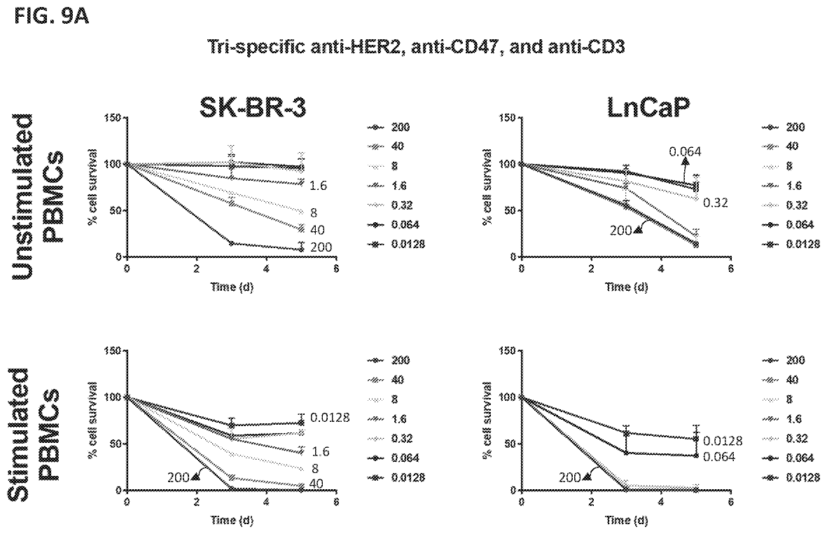

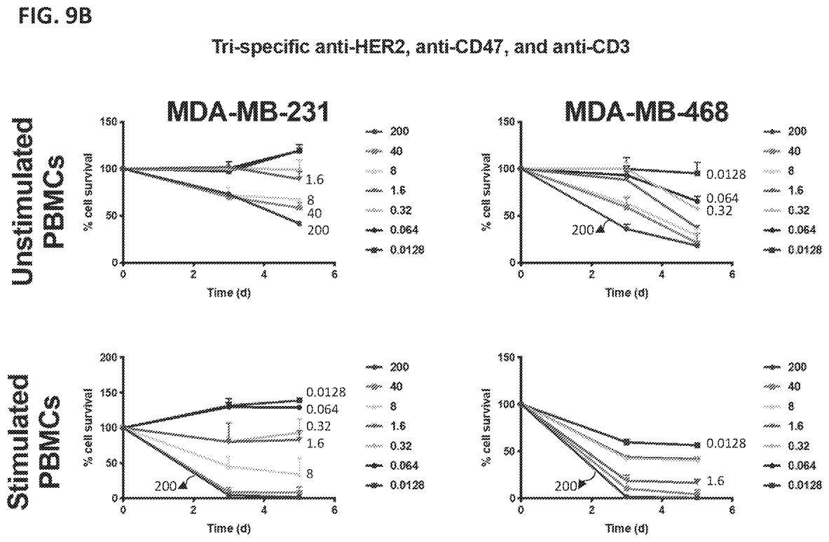

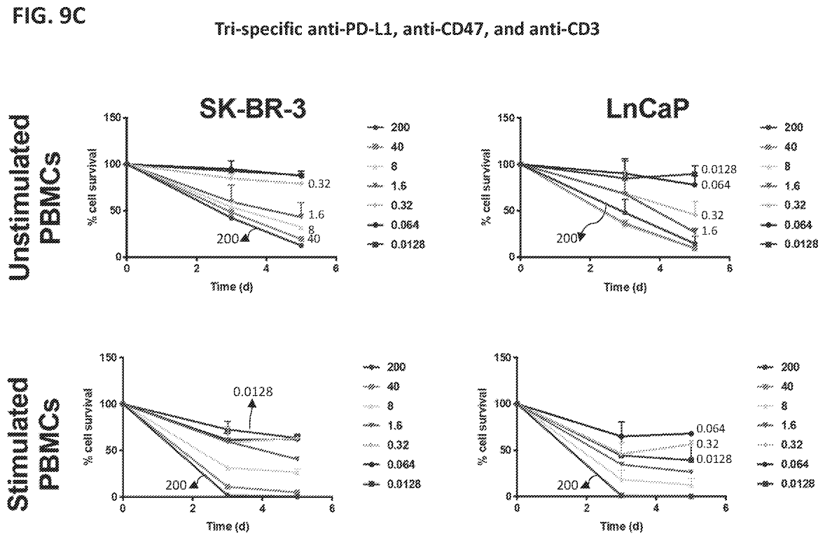

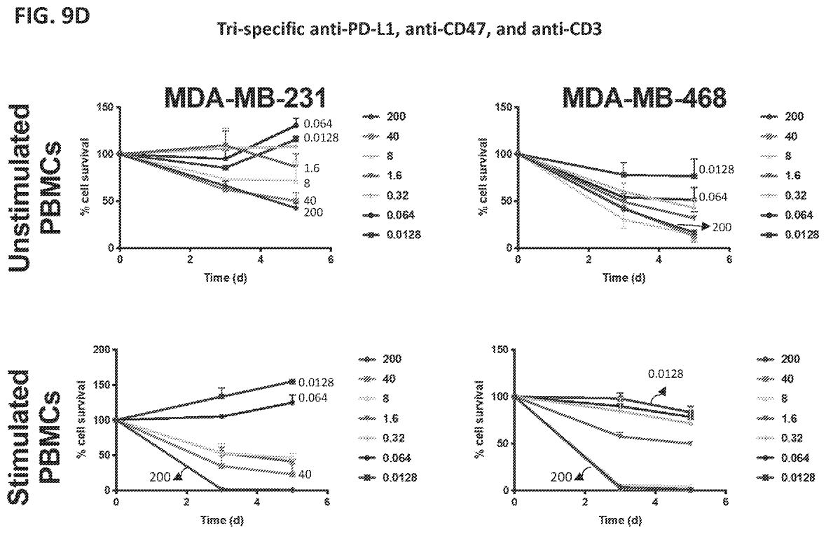

This example shows that antibody-mediated non-activated and activated T-cell mediated killing can be detected in multicellular 3D spheroids. Dual reporter SK-BR-3, LnCaP, MDA-MB-231, and MDA-MB-468 cells as constructed in Example 3 were plated into wells of a 96-well ultra-low attachment plate at 6,000 tumor cells/well in the presence of 6,000 human dermal fibroblast cells. The mixture of cells were incubated for 4 days to form 3D spheroids. Various antibodies (trispecific anti-PD-L1/anti-CD47/anti-CD3 antibody or trispecific anti-HER2/anti-CD47/anti-CD3 antibody) were added at different concentrations in the presence of 12,000 stimulated or unstimulated PBMCs (effector cells) to the 3D spheroids. To obtain different concentrations, antibodies were diluted 5-fold serially, from 200 ng/mL to 0.0128 ng/mL. The cell-killing reaction was incubated for 48 hours. After 48-hour incubation, doxycycline was added to the mixture of cells to induce expression of the dual reporters from live dual reporter tumor cells. Luminescence was monitored at different time points on a GloMax Discover Microplate Reader. The time course graphs were generated from two replicate measurements. As shown in FIGS. 9A-9D, antigen-dependent (HER2, PD-L1, and/or CD47) and antibody dose-dependent T-cell mediated killing in a multicellular 3D spheroid can be continuously monitored using the methods of the disclosure. As can be seen from FIGS. 9A-9D, higher antibody concentration and/or PBMC stimulation (versus non-stimulation) can result in stronger antibody-mediated effector cell killing on tumor cells formed in 3D spheroids.

The trispecific anti-HER2/anti-CD47/anti-CD3 antibody and trispecific anti-PD-L1/anti-CD47/anti-CD3 antibody (both made in house) have no Fc function due to LALA mutations and cannot mediate ADCC. Similar as discussed in Example 7, the inclusion of a CD47 antigen binding domain can allow the trispecific antibody to bypass HER2-dependent killing (e.g., compare antibody-mediated effector cell killing on MDA-MB-468 cells (no HER2 expression, high CD47 expression) in FIG. 9B and FIG. 7B) or PD-L1-dependent killing (e.g., compare antibody-mediated effector cell killing on MDA-MB-468 cells (no HER2, high CD47, no PD-L1) vs. MDA-MB-231 cells (low HER2, high CD47, high PD-L1) in FIG. 9D). The inclusion of a CD47 antigen binding domain might also allow the trispecific antibody to act synergistically with the HER2 or PD-L1 antigen binding domain to effect effector-mediated tumor cell killing.

### Example 9: Monitoring the Effect of Combined Cell-Killing Agents on T-Cell Function

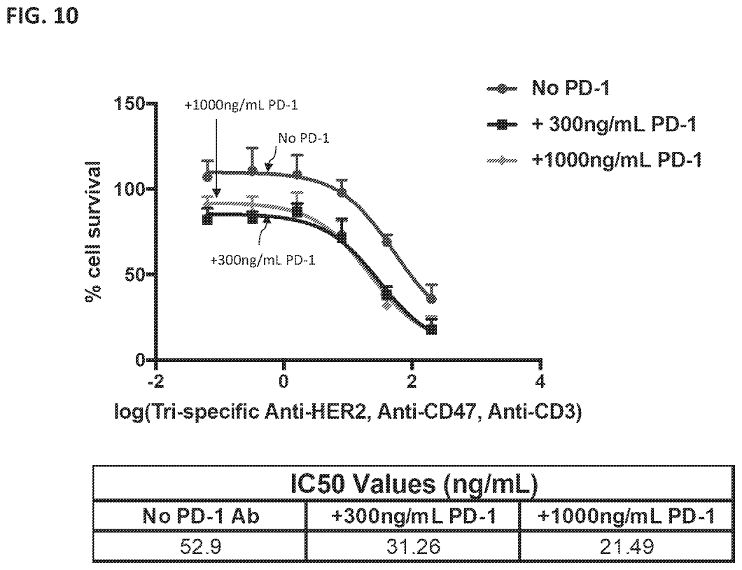

This example shows that the methods of the disclosure can quantify the effect of a combination of cell-killing agents (i.e., a combination therapy, such as, e.g., anti-PD-1 antibody and trispecific anti-HER2/anti-CD47/anti-CD3 antibody) on T-cell mediated tumor cell killing. Dual reporter MDA-MB-231 cells as constructed in Example 3 were plated into wells of a 96-well conical bottom plates at 5,000 cells/well in the presence of 5,000 unstimulated PBMCs. Different concentrations of trispecific anti-HER2/anti-CD47/anti-CD3 antibody (serially diluted 5-fold, 200 ng/mL-0.064 ng/mL) was added to each well with or without further addition of an anti-PD-1 antibody (300 ng/mL or 1000 ng/mL). The trispecific anti-HER2/anti-CD47/anti-CD3 antibody (made in house) has no Fc function due to LALA mutations and cannot mediate ADCC. The cell-killing reaction was incubated for 48 hours. After the 48-hour incubation, doxycycline was added to the mixture of cells to induce expression of the dual reporters from live dual reporter MDA-MB-231 cells. 24 hours after doxycycline induction, a cell culture media sample was taken, and luminescence was monitored on a GloMax Discover Microplate Reader. The dose-response curve was generated from three replicate measurements. As shown in FIG. 10, the results demonstrate that the methods of the disclosure can quantify the effect of combined cell-killing agents on effector cell-mediated tumor cell killing. As can be seen in FIG. 10, anti-HER2/anti-CD47/anti-CD3 antibody mediated T-cell killing on HER2+ MDA-MB-231 cells in an antibody-concentration dependent manner; anti-PD-1 antibody enhanced trispecific anti-HER2/anti-CD47/anti-CD3 antibody-mediated T cell killing on HER2+ MDA-MB-231 cells; and the more anti-PD-1 antibody was provided, the stronger the cytotoxicity. These findings are consistent with the results reported by Chang et al., Cancer Research 77 (19) 5384-94 (2017), which showed enhanced potency of anti-Trop-2/anti-CD3 bispecific antibodies mediated T cell killing on MDA-MB-231 spheroids in the presence of an anti-PD-1 antibody.

### Example 10: Monitoring T-Cell Mediated Tumor Cell Killing Kinetics in the Presence of Combined Cell-Killing Agents

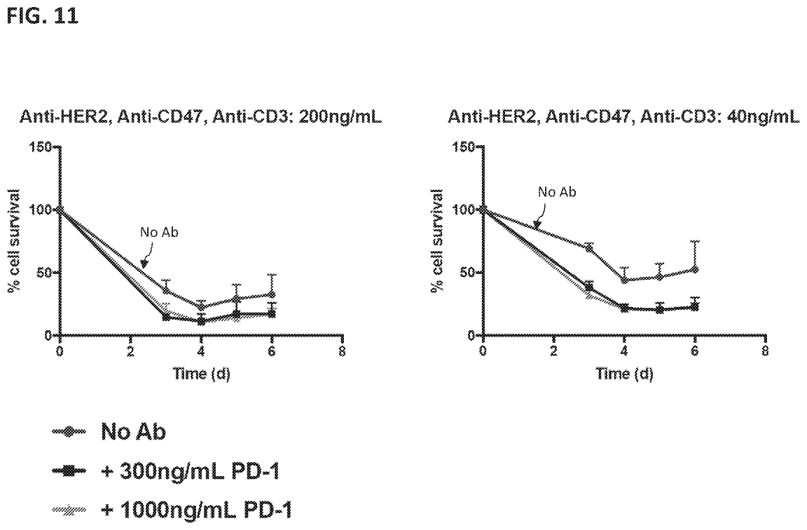

This example shows that the methods of the disclosure can quantify the effect of combination therapy (i.e. anti-PD-1 antibody and trispecific anti-HER2/anti-CD47/anti-CD3 antibody) on T-cell function over time. Dual reporter MDA-MB-231 cells as constructed in Example 3 were plated into wells of a 96-well conical bottom plate at 5,000 cells/well in the presence of 5,000 unstimulated PBMCs. Different concentrations of trispecific anti-HER2/anti-CD47/anti-CD3 antibody (200 ng/mL or 40 ng/mL) was added into each well with or without further adding different concentrations of anti-PD-1 antibody (300 ng/mL or 1000 ng/mL). The trispecific anti-HER2/anti-CD47/anti-CD3 antibody (made in house) has no Fc function due to LALA mutations and cannot mediate ADCC. The cell-killing reaction was incubated for 48 hours. After the 48-hour incubation, doxycycline was added to the mixture of cells to induce expression of the dual reporters from live dual reporter MDA-MB-231 cells. Cell culture media sample was taken at different time points post-induction, and luminescence was monitored on a GloMax Discover Microplate Reader. The time course was generated from three replicate measurements. As shown in FIG. 11, the results demonstrate that the methods of the disclosure can quantify the effect of combined cell-killing agents on effector cell-mediated tumor cell killing over time. As can be seen in FIG. 11, the higher concentration of anti-HER2/anti-CD47/anti-CD3 antibody, the stronger T-cell mediated killing on HER2+ MDA-MB-231 cells was seen; anti-PD-1 antibody enhanced trispecific anti-HER2/anti-CD47/anti-CD3 antibody-mediated T cell killing on HER2+ MDA-MB-231 cells; and the more anti-PD-1 antibody was provided, the stronger the cytotoxicity (see 40 ng/mL trispecific antibody panel).

### Example 11: The Total Reaction Time can Affect the Dose-Response Curve of Cell Killing

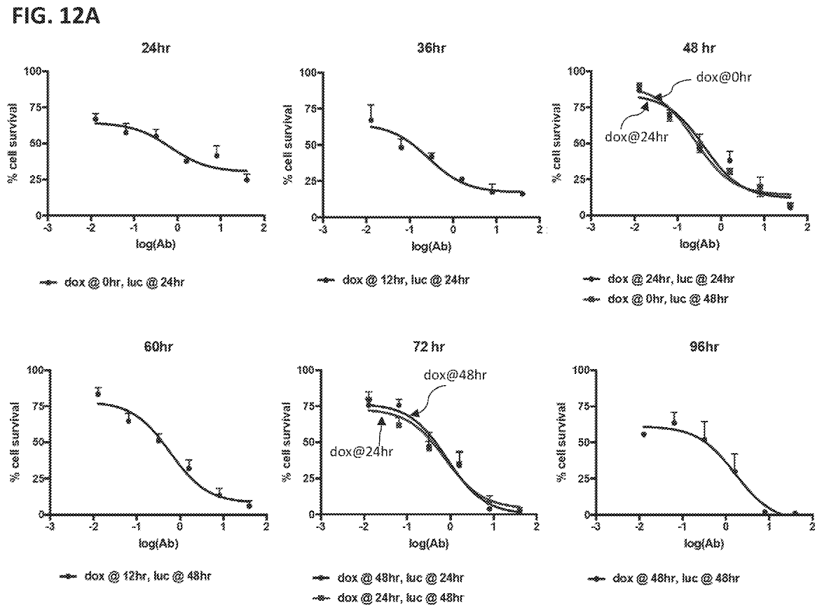

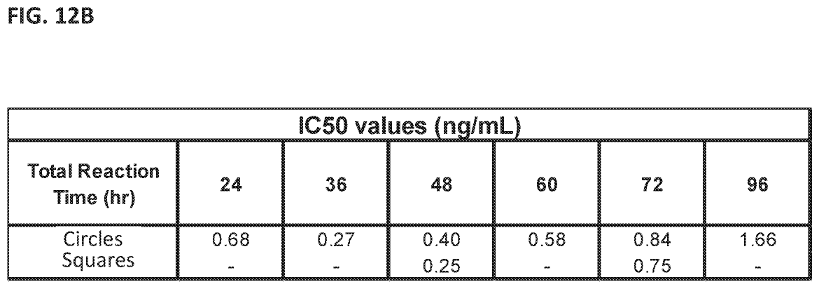

This example illustrates the benefit of using secreted inducible reporters in studying cell-mediated cytotoxicity. Since secreted reporter proteins accumulate in the media overtime, multiple measurements can be taken from the same sample well. This allows us to analyze how cell-mediated cytotoxicity changes over the total reaction time and under different reporter induction timing. 15,000 unstimulated PBMCs and 5,000 dual reporter SK-BR-3 cells as constructed in Example 2 (3E:1T) were plated in a 96-well conical bottom plate with different concentrations of Herceptin® antibody (serially diluted 5-fold, 200 ng/mL-0.0128 ng/mL). The timing of the reporter expression phase was changed by adding doxycycline at different time points: 0, 12, 24, and 48 hours post-incubation of antibody, tumor cells, and PBMCs (dox@0, 12, 24, or 48 hr in FIG. 12A). snLuciferase was measured 24 and 48 hours after adding doxycycline (luc@t24 hr and luc@48 hr in FIG. 12A). Total reaction time was calculated as the incubation time before adding doxycycline, plus doxycycline induction time before measuring snLuciferase (indicated on top of each panel in FIG. 12A). The dose-response curves were generated from two replicates. As shown in FIGS. 12A-12B, the results demonstrate that the dose-response curves can change over time. If the total reaction time is too short, the detected cell-killing effect could be weak even if report expression is induced early on, because not enough time has passed for cytolysis to occur (compare luc@24 hr and dox@0 hr with others in FIGS. 12A-12B). If the total reaction time is too long, even if cytolysis has enough time to occur, the detected cell-killing effect could be weak because the majority of target tumor cells have already been lysed (compare dox@48 hr and luc@48 hr with others in FIGS. 12A-12B). Therefore, there is a fine balance between the total reaction time, the timing of inducing reporter expression, and the timing of reporter protein detection. The inducible reporter system described herein allows us to optimize the experimental conditions and select for the time in which cytotoxicity is maximized, resulting in a highly sensitive and versatile assay.

### Example 12: Dual Report Expression Level Correlates with Live Tumor Cell Number when Co-Cultured with Primary Unstimulated T Cells

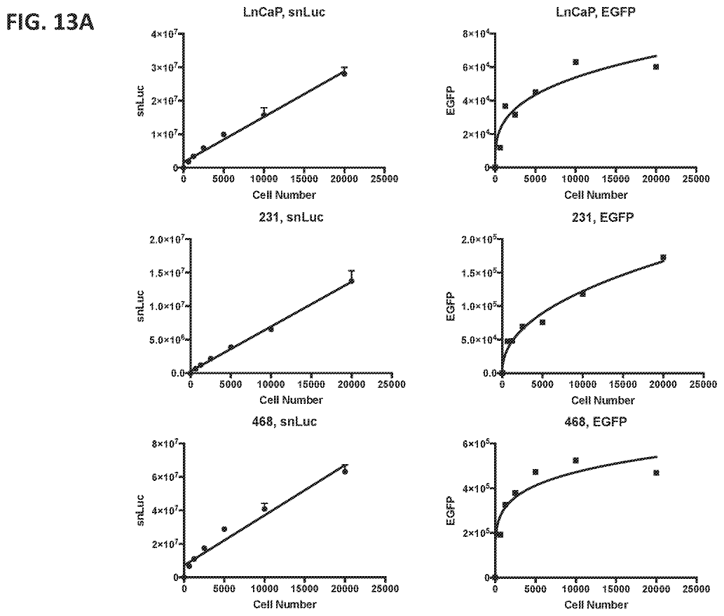

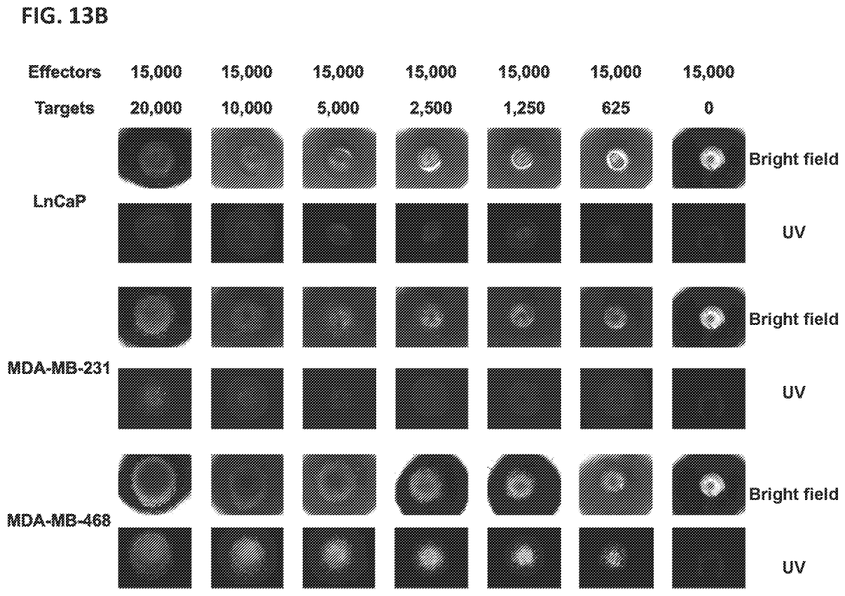

Various concentrations of dual reporter LnCaP, MDA-MB-231, and MDA-MB-468 cells as constructed in Example 3 were plated in a non-cell culture treated 96-well conical bottom plate with 15,000 unstimulated, primary T cells. No further cell-killing agent was added. Dual reporter tumor cells were serially diluted 2-fold, ranging from 0-20,000 tumor cells per well. Doxycycline was added to the mixture of cells immediately after plating, to induce expression of the dual reporters. 24 hours after doxycycline induction, the dual reporter tumor cells were analyzed using a Nikon Ellipse TE2000-U microscope for EGFP (and bright field as experimental condition and cell number controls), and snLuciferase was measured using a GloMax Discover Microplate Reader. As can be seen from FIGS. 13A-13B, snLuciferase and EGFP signal correlated with live dual reporter tumor cells, of which snLuciferase linearly correlated to live dual reporter tumor cell number from 0-20,000, and EGFP linearly correlated to live dual reporter tumor cell number from 0-5,000. Similar to results from Example 5, the results here demonstrate that both snLuciferase and EGFP can be used to quantify live tumor cell number, while snLuciferase may serve as a more sensitive semi-quantitative marker than EGFP (EGFP has a limited linear range compared to snLuciferase measurement).

### Example 13: Optimizing Reporter Induction Time in Effector-Cell Mediated Tumor Cell Killing Assays

This example illustrates how timing of the beginning of the reporter expression phase can affect effector cell-mediated tumor cell killing, and how to optimize experimental conditions of the invention.

15,000 unstimulated PBMCs and 5,000 dual reporter MDA-MB-231 cells as constructed in Example 3 (3E:1T) were plated in a 96-well conical bottom plate with different concentrations of trispecific anti-HER2/anti-CD47/anti-CD3 antibody (serially diluted 5-fold, 200 ng/mL-0.0128 ng/mL). Control wells did not add antibody. The trispecific anti-HER2/anti-CD47/anti-CD3 antibody (made in house) has no Fc function due to LALA mutations and cannot mediate ADCC. The timing of the reporter expression phase was changed by adding doxycycline at different time points: −24 hours (dox induction in dual reporter MDA-MB-231 cells 24 hours before co-incubating antibody/tumor cells/PBMCs, to mimic “constitutive” expression), and 0, 24, 48, and 72 hours post-incubation with antibody. 24 hours after doxycycline induction, media was taken from each well and snLuciferase luminescence was measured using a GloMax Discover Microplate Reader. The dose-response curves were generated from three replicates. Percent cell survival was defined as snLuciferase readout (RLU) at various antibody concentrations relative to the average readout with no antibody.

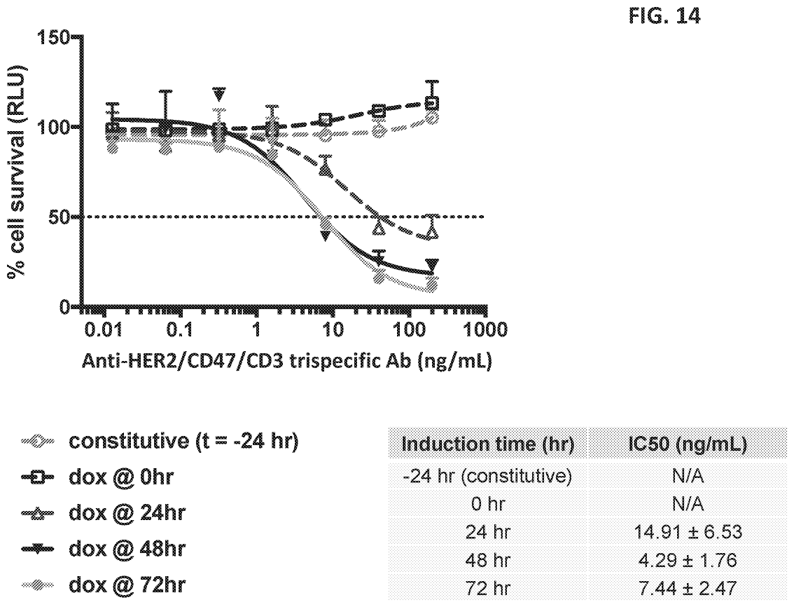

As shown in FIG. 14, no cytotoxic T cell mediated tumor cell killing was observed in a “constitutive” expression system where dual reporter expression was induced 24 hours before contacting tumor cells with anti-HER2/anti-CD47/anti-CD3 trispecific antibody and PBMCs, or when doxycycline was added simultaneously with antibody/tumor cell/effector cell incubation (dox@0 hr). However, an antibody dose-dependent cell killing curve was observed when doxycycline was added 24, 48, and 72 hours post-incubation of antibody/tumor cell/effector cell. These results demonstrate that by controlling when the reporter proteins are expressed from tumor cells and total reaction time, we can optimize the experimental conditions and select for the time in which cytotoxicity is maximized, resulting in a highly sensitive and versatile assay. Optimization of total reaction time and doxycycline induction timing should be determined based on timing of two stages, an effecting stage, which is the time when majority of target tumor cells are cytolyzed; and a detection stage, which is the time when reporters are measured.

FIG. 14 demonstrates that an inducible reporter system of the present invention is superior to a constitutive expression system (e.g., those under promoter control of EF1-α or CMV etc.). If snLuciferase is induced before cytolysis has enough time to occur, the majority of target cells would be viable and secrete snLuciferase which accumulates over time in the media, thus skewing the final RLU readout. This would results in complete loss of detected tumor cell killing (see “constitutive (t=−24 hr)” and dox@0 hr). Therefore, an inducible reporter system of the present invention allows us to reduce background expression associated with secreted reporter proteins.

Further, it is not the later the induction of reporter expression the stronger the detected cytotoxicity. As seen in FIG. 14, maximum cytotoxicity was reached when doxycycline was added 48 hours post-incubation of antibody/tumor cell/effector cell (dox@48 hr), earlier induction when not enough cytolysis has happened (dox@24 hr) or later induction when more or majority of cells have been lysed (dox@72 hr) both showed less cytotoxicity effect. Thus, expressing reporter proteins under an inducible system allows us to optimize and select for the time when cytotoxicity is maximized, resulting in a highly sensitive and versatile assay.

### Example 14: Tumor Antigen Expression Level Affects Antibody-Mediated Effector Cell Killing on Tumor Cells

This example evaluates the effect of tumor antigen expression level on effector-cell mediated cell killing.

15,000 unstimulated PBMCs and 5,000 dual reporter tumor cells (LnCaP, MDA-MB-231, and MDA-MB-468) as constructed in Example 3 (3E:1T) were plated in a 96-well conical bottom plate with different concentrations of bispecific anti-HER2/anti-CD3 antibody or trispecific anti-HER2/anti-CD47/anti-CD3 antibody (serially diluted 5-fold, 200 ng/mL-0.064 ng/mL). Control wells did not add antibody. The bispecific anti-HER2/anti-CD3 antibody and trispecific anti-HER2/anti-CD47/anti-CD3 antibody (both made in house) has no Fc function due to LALA mutations and cannot mediate ADCC. The cell-killing reaction was incubated for 48 hours. After the 48-hour incubation, doxycycline was added (t=48 hr) to the mixture of cells to induce expression of the dual reporters from live dual reporter tumor cells. Cell culture media was taken one day post-induction with doxycycline (t=72 hr), and luminescence was measured on a GloMax Discover Microplate Reader. Percent cell survival was defined as luminescence readout at various antibody concentrations relative to the average readout with no antibody. The dose-response curves were generated from three replicate measurements.

Expression of tumor antigens (HER2, CD47, PD-L1) were measured using FACS. Briefly, tumor cells were incubated with Herceptin® (secondary staining with APC anti-human IgG), Alexa Fluor® 647 anti-human CD47 clone CC2C6 (BioLegend, cat #323117), or PE anti-human PD-L1 clone MIH3 (BioLegend, cat #374511) for 45 minutes at 4° C., and washed three times before analysis with Guava® easyCyte. Tumor antigen expression level is summarized in FIG. 15C: LnCaP (intermediate HER2, high CD47, no PD-L1); MDA-MB-231 (low HER2, high CD47, high PD-L1); MDA-MB-468 (no HER2, high CD47, no PD-L1).

As shown in FIGS. 15A and 15D, bispecific anti-HER2/anti-CD3 antibody mediate T-cell killing in an antibody concentration-dependent and antigen expression level-dependent manner—the higher expression of tumor antigen (HER2, see FIG. 15C), and/or the higher concentration of the antibody, the stronger the T cell-mediated tumor cell killing can be detected. This suggests that the methods described in the invention can detect antigen-dependent cell killing.

As shown in FIGS. 15B and 15D, the addition of an anti-CD47 antigen binding domain to anti-HER2/anti-CD3 antibody strengthened T cell-mediated tumor cell killing on LnCaP cells (intermediate HER2, high CD47) and MDA-MB-231 cells (low HER2, high CD47), and bypassed HER2-dependent killing on MDA-MB-468 cells (no HER2, high CD47). MDA-MB-231 cells were found to exhibit certain resistance to antibody-mediated T cell killing, likely because of high PD-L1 expression on MDA-MB-231 cells compared to others (FIG. 15C).

### Example 15: Effector to Target Cell Ratios Affect Antibody-Mediated Cell Killing on Dual Reporter Tumor Cells

Unstimulated PBMCs or T cells from different patient donors and dual reporter MDA-MB-231 cells as constructed in Example 3 were plated at different E:T ratios in a 96-well conical bottom plate, with different concentrations of bispecific anti-HER2/anti-CD3 antibody (serially diluted 5-fold, 200 ng/mL-0.0128 ng/mL). Control wells did not add antibody. The bispecific anti-HER2/anti-CD3 antibody (made in house) has no Fc function due to LALA mutations and cannot mediate ADCC. The cell-killing reaction was incubated for 48 hours. After the 48-hour incubation, doxycycline was added (t=48 hr) to the mixture of cells to induce expression of the dual reporters from live tumor cells. Cell culture media was taken one day post-induction with doxycycline (t=72 hr), and luminescence was measured on a GloMax Discover Microplate Reader. Percent cell survival was defined as luminescence readout at various antibody concentrations relative to the average readout with no antibody. The dose-response curves were generated from three replicate measurements.

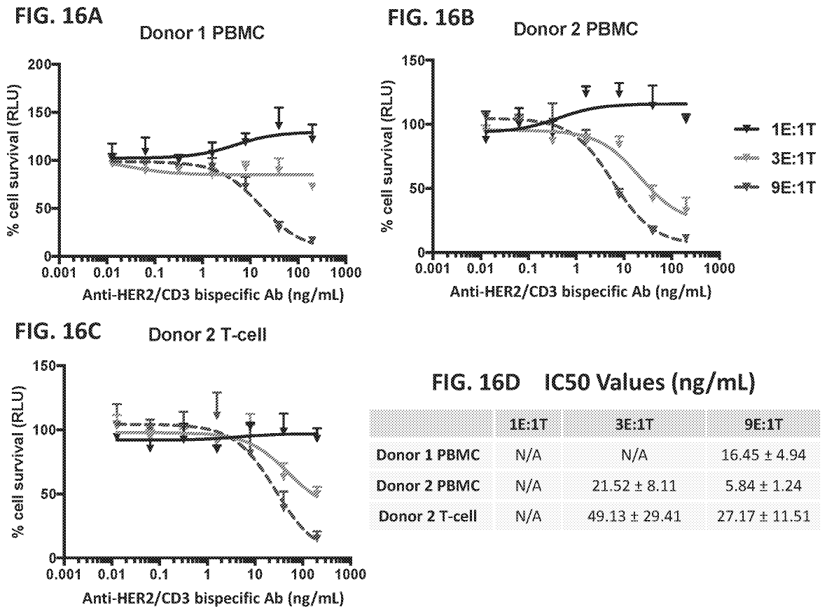

As shown in FIGS. 16A-16D, E:T ratios greatly affect antibody-mediated effector cell killing—the higher E:T ratio, the stronger the cytotoxicity. None of the patients showed CTL killing at a 1E:1T ratio, partial response was seen at 3E:1T ratio (donors 2 and 3 showed CTL killing, but donor 1 did not), and all patients showed CTL killing at 9E:1T. This result is consistent with the finding that tumor microenvironment (e.g., percentage of tumor-infiltrating lymphocyte (TIL) in tumor) can drastically affect patient response to immunotherapy, and higher tumor-infiltration correlates to better clinical outcomes to immunotherapy. The results here also demonstrate that the methods described in the invention can detect difference among patients, e.g., although both donor 1 and donor 2 were tested with PBMCs, only donor 2 responded to antibody-mediated CTL killing. Further, comparing to Example 14 (3E:1T), results here demonstrate that this assay can detect individual donor-to-donor immune-cell differences, and increasing E:T ratio can bypass immune suppression observed in MDA-MB-231 (high PD-L1 expression).

### Example 16: Stimulated T Cells Cannot Overcome Immunosuppression Observed in Dual Reporter MDA-MB-231 Cells with High PD-L1 Expression

Mixture of unstimulated and stimulated T cells (effector) with various contents of stimulated T cells (0% stimulated to 100% stimulated T cells) and dual reporter tumor cells (MDA-MB-231 and MDA-MB-468) as constructed in Example 3 were plated at 1E:1T ratio in a 96-well conical bottom plate, with different concentrations of trispecific anti-HER2/anti-CD47/anti-CD3 antibody (serially diluted 5-fold, 100 ng/mL-0.0064 ng/mL). Control wells did not add antibody. The trispecific anti-HER2/anti-CD47/anti-CD3 antibody (made in house) has no Fc function due to LALA mutations and cannot mediate ADCC. The cell-killing reaction was incubated for 48 hours. After the 48-hour incubation, doxycycline was added (t=48 hr) to the mixture of cells to induce expression of the dual reporters from live tumor cells. Cell culture media was taken one day post-induction with doxycycline (t=72 hr), and luminescence was measured on a GloMax Discover Microplate Reader. Percent cell survival was defined as luminescence readout at various antibody concentrations relative to the average readout with no antibody. The dose-response curves were generated from three replicate measurements.

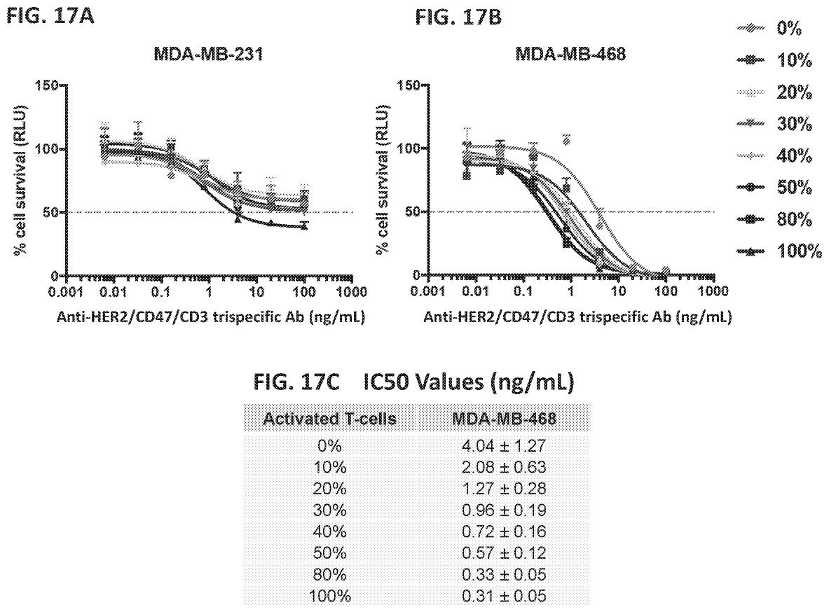

As can be seen from FIG. 17A, no significant difference in antibody-mediated T cell killing was observed against MDA-MB-231 cells with varying ratios of stimulated vs. unstimulated T-cells. However, antibody-mediated T cell killing (quantified using IC50 values) against MDA-MB-468 cells increased as the number of stimulated T-cells increased in the mixture of T cells (FIGS. 15B-15C). As shown in FIG. 15C, MDA-MB-231 cells have high PD-L1 expression, while MDA-MB-468 cells have no PD-L1 expression. These results suggest that PD-1/PD-L1 pathway might block CTL activity on tumor cells. This is consistent with clinical data (Alsaab et al., Front Pharmacol. 2017; 8:561), which suggests that PD-1/PD-L1 pathway can block CD8+ T-cell effector function.

### Example 17: Modulating PD-1/PD-L1 Blockade can Affect Effector Cell-Mediated Tumor Cell Killing

This example evaluates PD-1/PD-L1 blockade on CTL activity in vitro and rescue effect of nivolumab (anti-PD-1 antibody) on CTL activity from PD-1/PD-L1 blockade.

Dual reporter MDA-MB-231 cell line with PD-L1 knockout (KO) (hereinafter referred to as “dual reporter MDA-MB-231 PD-L1 KO cells” or “MDA-MB-231 KO”) was constructed by co-transducing the Tet-on snLuc-GFP construct as constructed in Example 1 and CRISPR/Cas9 constructs targeting PD-L1. 3D tumor-fibroblast spheroids were generated by co-culturing 10,000 human dermal fibroblasts and 10,000 dual reporter MDA-MB-231 cells (“MDA-MB-231 WT”) or dual reporter MDA-MB-231 PD-L1 KO cells in ultra-low attachment plates for 3 days. After spheroid formation, 30,000 unstimulated primary T cells (3E:1T) were added in the presence of increasing concentrations of trispecific anti-HER2/anti-CD47/anti-CD3 antibody (serially diluted 5-fold, 1000 ng/mL-0.032 ng/mL), and without or with anti-PD-1 antibody nivolumab (0.5 μg/mL). Control wells did not add antibody.

Dual reporter MDA-MB-468 cell line overexpressing PD-L1 (hereinafter referred to as “dual reporter MDA-MB-468 PD-L1 overexpressing cells”) was constructed by co-transducing the Tet-on snLuc-GFP construct as constructed in Example 1 and an PD-L1 expression construction under CMV promoter control. 10,000 dual reporter MDA-MB-468 cells (“MDA-MB468 WT”) or dual reporter MDA-MB-468 PD-L1 overexpressing cells were co-cultured with 30,000 unstimulated primary T-cells (3E:1T) in non-treated, conical bottom plate in the presence of increasing concentrations of trispecific anti-HER2/anti-CD47/anti-CD3 antibody (serially diluted 5-fold, 200 ng/mL-0.0032 ng/mL). Control wells did not add antibody.

The trispecific anti-HER2/anti-CD47/anti-CD3 antibody (made in house) has no Fc function due to LALA mutations and cannot mediate ADCC. The cell-killing reaction was incubated for 48 hours. After the 48-hour incubation, doxycycline was added (t=48 hr) to the mixture of cells to induce expression of the dual reporters from live tumor cells. Cell culture media was taken one day post-induction with doxycycline (t=72 hr), and luminescence was measured on a GloMax Discover Microplate Reader. Percent cell survival was defined as luminescence readout at various antibody concentrations relative to the average readout with no antibody. The dose-response curves were generated from three replicate measurements.

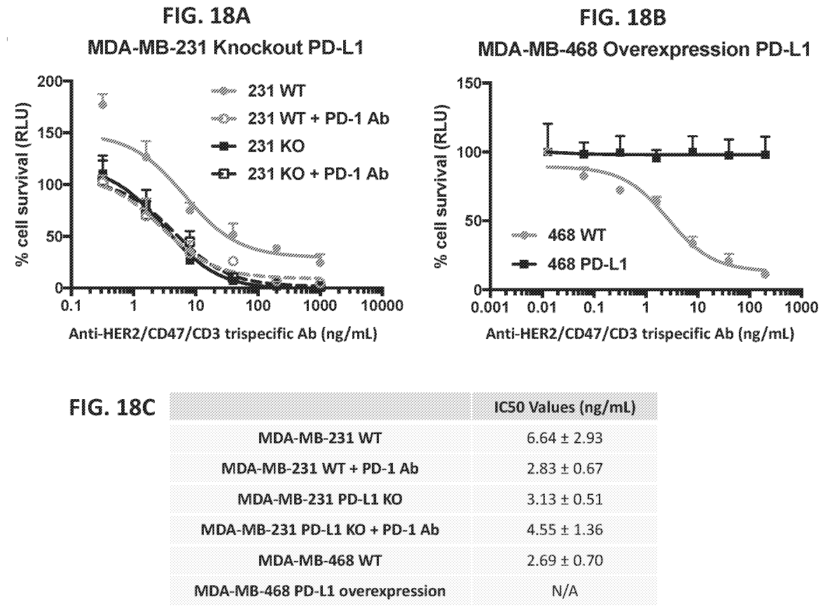

As can be seen in FIGS. 18A and 18C, PD-L1 KO significantly enhanced antibody-mediated T cell killing on dual reporter MDA-MB-231 cells (compare “231 WT” and “231 KO”). Co-incubation with anti-PD-1 antibody nivolumab rescued T cell killing on dual reporter MDA-MB-231 cells (compare “231 WT+PD-1Ab” and “231 WT”), and the cytotoxicity rescue effect was similar or even better than T-cell cytotoxicity seen in PD-L1 KO (compare “231 WT+PD-1 Ab” and “231 KO”). These results also suggest that methods described in the invention can be used for detecting effector cell-mediated killing in 3D tumor-fibroblast spheroids model. As can be seen in FIGS. 18B and 18C, PD-L1 overexpression in MDA-MB-468 cells abolished antibody-mediated T cell killing effect (compare “468 WT” and “468 PD-L1”). These results demonstrate that modulating PD-1/PD-L1 blockade can affect effector cell-mediated tumor cell killing.

To summarize, an assay system that can mimic the immunosuppression observed in in vivo tumor microenvironment has been generated. The inducible reporter assays described herein can enable sensitive analysis of the immunosuppressive effect of PD-1/PD-L1 blockade on CTL killing. Further, it was demonstrated that the addition of anti-PD-1 antibody (nivolumab, e.g., Opdivo®) could rescue the immunosuppression on cytotoxic T cells. Thus, the system described herein can provide the opportunity to screen for new and/or improved immunotherapy candidates in a sensitive and high-throughput manner.

### Example 18: Optimizing Reporter Induction Time in ADCC Mediated by NK Cells

This example illustrates how timing of the beginning of the reporter expression phase can affect ADCC mediated by NK cells, and how to maximize ADCC using the inducible reporter system.

15,000 NK92 (CD16+) cells and 5,000 dual reporter SK-BR-3 cells as constructed in Example 3 (3E:1T) were plated in a 96-well conical bottom plate with different concentrations of anti-HER2 antibody trastuzumab (Herceptin®; serially diluted 5-fold, 200 ng/mL-0.0128 ng/mL). Control wells did not add antibody. The timing of the reporter expression phase was changed by adding doxycycline at different time points: −24 hours (dox induction in dual reporter SK-BR-3 cells 24 hours before co-incubating antibody/tumor cells/NK92, to mimic “constitutive” expression), and 0, 4, and 12 hours post-incubation of antibody/tumor cells/NK92. 24 hours after doxycycline induction, media was taken from each well and snLuciferase luminescence was measured using a GloMax Discover Microplate Reader, and Nikon Ellipse TE2000-U microscope was used to capture EGFP signal. The dose-response curves were generated from three replicates. Percent cell survival was defined as snLuciferase readout (RLU) at various antibody concentrations relative to the average readout with no antibody.

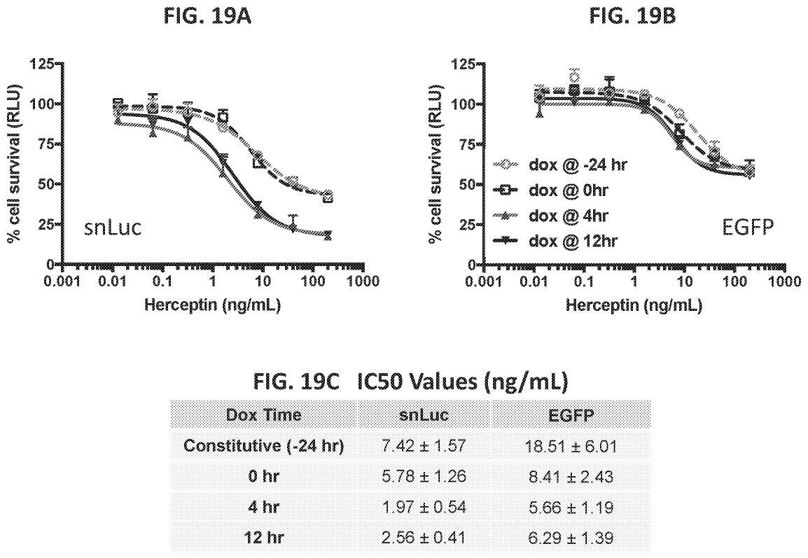

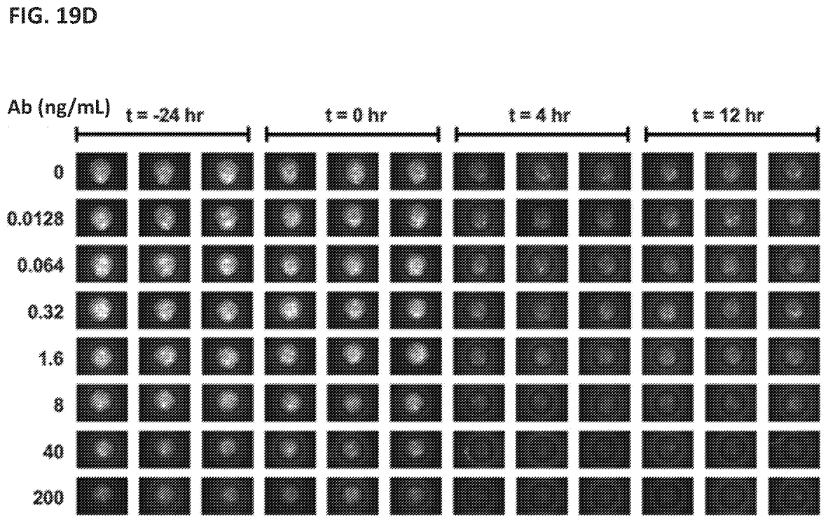

As can be seen from FIGS. 19A-19C, snLuciferase may serve as a more sensitive semi-quantitative marker than EGFP. FIGS. 19A-19D show that NK cell ADCC is mediated in an antibody-concentration dependent manner, and that doxycycline added at 4 hrs or 12 hrs post-incubation of antibody/tumor cell/effector cell could achieve stronger ADCC compared to a “constitutive” expression system represented by inducing dual reporter expression at least 24 hours before contacting the tumor cells with trastuzumab and NK92, or compared to when doxycycline was added simultaneously with antibody/tumor cell/effector cell incubation (dox@0 hr). These results demonstrate that by controlling total reaction time and when the reporter proteins are expressed from tumor cells, we can optimize the experimental condition and select for the time in which cytotoxicity is maximized, resulting in a highly sensitive and versatile assay.

### Example 19: Tumor Antigen Expression Level Affects ADCC Mediated by NK Cells

This example evaluates the effect of tumor antigen expression level on NK-cell mediated ADCC.

15,000 NK92 (CD16+) cells and 5,000 dual reporter tumor cells (SK-BR-3, LnCaP, MDA-MB-231, MCF-7, and MDA-MB-468) as constructed in Example 3 (3E:1T) were plated in a 96-well conical bottom plate with different concentrations of anti-HER2 antibody trastuzumab (Herceptin®; serially diluted 5-fold, 1000 ng/mL-0.32 ng/mL). Control wells did not add antibody. After 8-hour incubation, doxycycline was added (t=8 hr) to the mixture of cells to induce expression of the dual reporters. Cell culture media was taken one day post-incubation of antibody/tumor cells/NK cells (t=24 hr), and luminescence was measured on a GloMax Discover Microplate Reader. Percent cell survival was defined as luminescence readout at various antibody concentrations relative to the average readout with no antibody. The dose-response curves were generated from three replicate measurements.

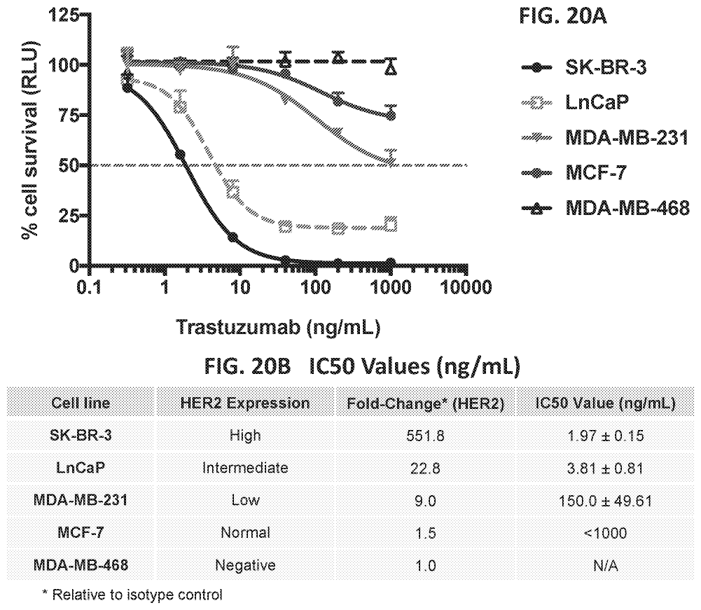

Expression of tumor antigen (HER2) was measured using FACS. Briefly, tumor cells were incubated with anti-HER2 antibody trastuzumab (Herceptin®) for 45 minutes at 4° C. and washed three times. Cells were incubated with a secondary antibody targeting anti-human IgG1 for 30 minutes at 4° C., and washed three times before analysis with Guava® easyCyte. Tumor antigen expression levels of various cancer cell lines were normalized to that of MDA-MB-468, which does not express HER2 and served as control (FIG. 20B).

As shown in FIGS. 20A-20B, ADCC mediated by NK cells was in an antibody concentration-dependent and antigen expression level-dependent manner—the higher expression of tumor antigen (HER2, see FIG. 20B), and/or the higher concentration of the anti-HER2 antibody trastuzumab, the stronger the NK cell-mediated ADCC on tumor cells can be detected. Dual reporter MDA-MB-468 cells which do not express HER2 served as a control without observed ADCC. These results suggest that the methods described in the invention can detect antigen-dependent ADCC in a sensitive manner, which are superior to other known ADCC assays which usually require very high antigen expression levels on tumor target cells.

### Example 20: Effector to Target Cell Ratios Affect ADCC on Dual Reporter Tumor Cells

Unstimulated PBMCs from different patient donors and dual reporter SK-BR-3 cells as constructed in Example 3 were plated at various E:T ratios in a 96-well conical bottom plate, with different concentrations of anti-HER2 antibody trastuzumab (Herceptin®; serially diluted 5-fold, 1000 ng/mL-0.32 ng/mL). Control wells did not add antibody. After 8-hour incubation, doxycycline was added (t=8 hr) to the mixture of cells to induce expression of the dual reporters. Cell culture media was taken one day post-incubation of antibody/tumor cells/PBMCs (t=24 hr), and luminescence was measured on a GloMax Discover Microplate Reader. Percent cell survival was defined as luminescence readout at various antibody concentrations relative to the average readout with no antibody. The dose-response curves were generated from three replicate measurements.

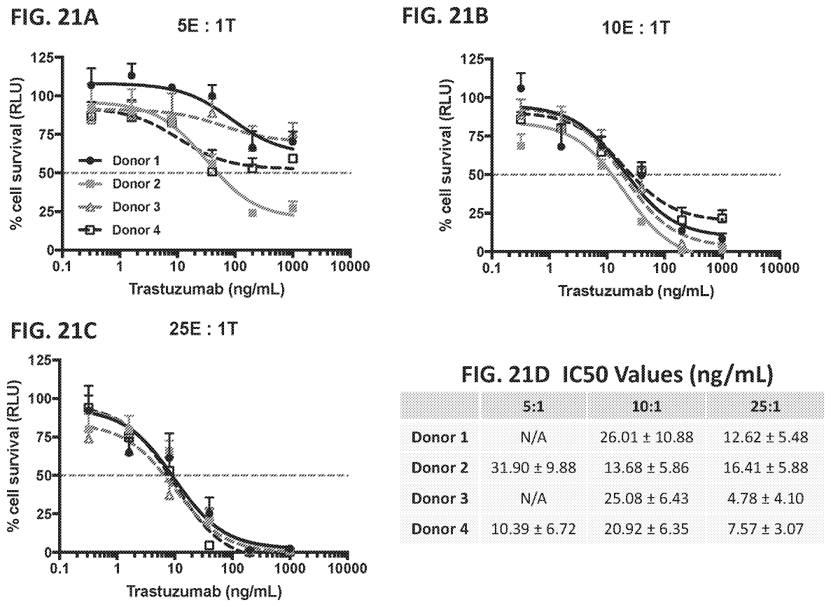

As shown in FIGS. 21A-21D, E:T ratios greatly affect trastuzumab-mediated ADCC by PBMCs—the higher E:T ratio, the stronger the ADCC (denoted by IC50). Partial response was seen at 5E:1T ratio (donors 2 and 4 showed ADCC, but donors 1 and 3 did not), and all patients showed ADCC at 10E:1T and 25E:1T. This result is consistent with the finding that tumor microenvironment (e.g., percentage effector immune cells in tumor) can drastically affect patient response to immunotherapy, and higher tumor-infiltration correlates to better clinical outcomes to immunotherapy. The results here also demonstrate that the methods described in the invention can detect difference among patients, see donors 2 and 4 showed different levels of ADCC by PBMCs, but donors 1 and 3 did not. Experimental condition should be designed with the notion in mind that higher E:T ratio might also result in higher levels of non-specific killing.

### Example 21: Quantification of ADCC of Trastuzumab with the Presence of Cancer Patient Serum

15,000 NK92 (CD16+) cells and 5,000 dual reporter SK-BR-3 cells as constructed in Example 3 (3E: T) were plated in a 96-well conical bottom plate with different concentrations of anti-HER2 antibody trastuzumab (Herceptin®; serially diluted 2-fold, 1000 ng/mL-0.122 ng/mL), in either culture medium containing 10% FBS (“control”) or culture medium with a 1/10 final dilution of a human cancer patient serum (“serum”). Control wells did not add antibody. After 24-hour incubation, doxycycline was added (t=24 hr) to the mixture of cells to induce expression of the dual reporters from live tumor cells. Cell culture media was taken one day post-induction with doxycycline (t=48 hr), and luminescence was measured on a GloMax Discover Microplate Reader. Percent cell survival was defined as luminescence readout at various antibody concentrations relative to the average readout with no antibody. The dose-response curves were generated from three replicate measurements.

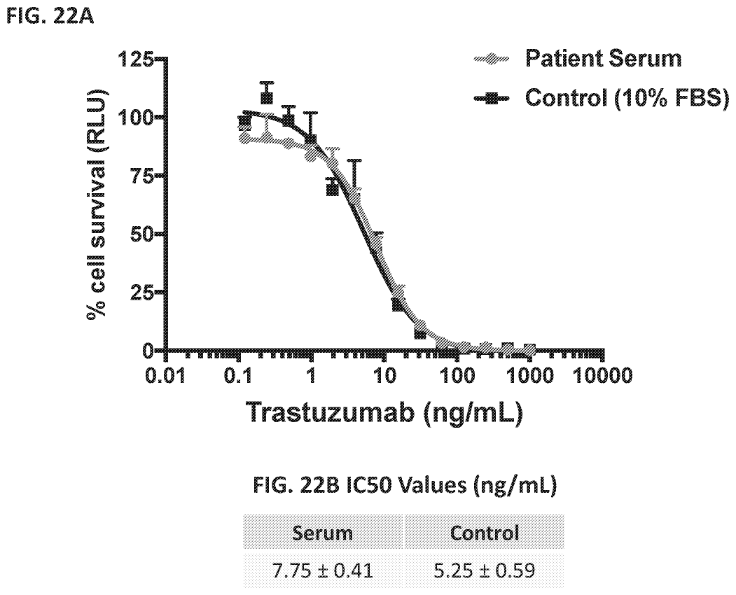

Patient serum contains a mixture of IgG which can compete for CD16 binding on NK cells, thus most ADCC assays use a higher 1/20 dilution. As shown in FIGS. 22A-22B, NK cell-mediated ADCC activity on dual reporter tumor cells could be detected even with high amount of patient serum (1/10 dilution), and that ADCC measured with the presence of control serum and patient serum did not significantly differ (see FIG. 22B). This suggests that the methods and assay system described in the invention can serve as a useful clinical tool in detecting ADCC in patient serum.

### Example 22: Evaluation of ADCC of Trastuzumab in 3D Tumor Spheroids

3D tumor spheroids were generated by culturing 10,000 dual reporter LnCaP cells as constructed in Example 3 in ultra-low attachment plates for 3 days. After spheroid formation, 30,000 NK92 (CD16+) cells (3E: T) were added in the presence of increasing concentrations of anti-HER2 antibody trastuzumab (Herceptin®; serially diluted 5-fold, 1000 ng/mL-0.32 ng/mL). Control wells did not add antibody. After 12-hour incubation, doxycycline was added (t=12 hr) to the reaction to induce expression of the dual reporters from live tumor cells. Cell culture media sample was taken every day post-induction with doxycycline (t=24, 48, and 72 hrs), and luminescence was measured on a GloMax Discover Microplate Reader. EGFP signal was monitored at the same time points using a Nikon Ellipse TE2000-U microscope. Percent cell survival was defined as signal readout at various antibody concentrations relative to the average readout with no antibody. The dose-response curves were generated from three replicate measurements.

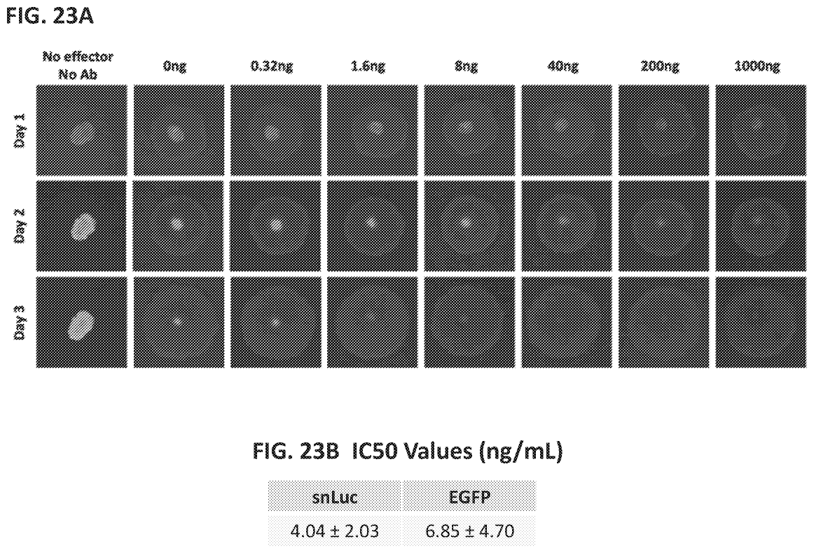

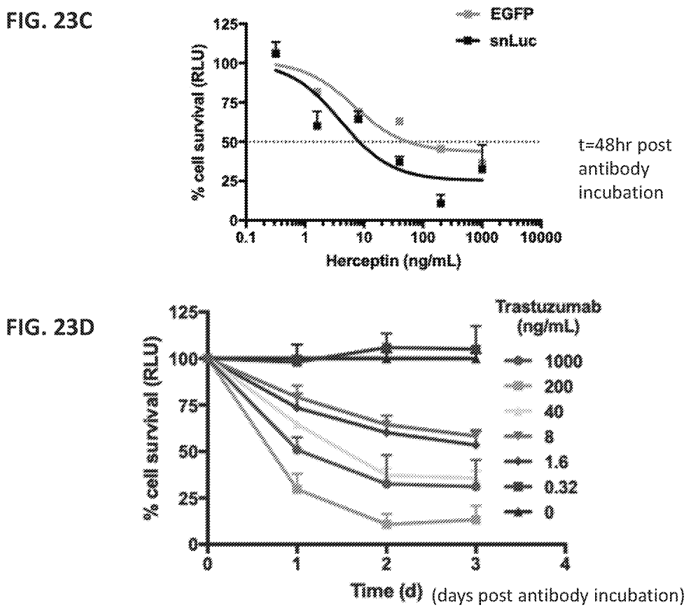

FIGS. 23A and 23C demonstrate NK cell-mediated ADCC can be visualized through changes in fluorescent signal EGFP over time. FIGS. 23C-23D demonstrate that NK cell-mediated ADCC can be visualized through changes in snLuciferase signal in an antibody concentration dependent manner, and/or overtime (FIG. 23D). Further, FIGS. 23B-23C suggest that snLuciferase may serve as a more sensitive semi-quantitative marker than EGFP. These results demonstrate that the methods of the invention can be used to detect NK cell-mediated ADCC in a sensitive manner in 3D tumor spheroid model.

To summarize, examples provided here demonstrate that CD8+ T-cells (CTLs) and natural killer (NK) cells play a key role in anti-cancer immune responses. Cell-contact-dependent cytotoxicity is a hallmark of T-cell and NK cell responses. Here, we have developed a cell-based cytotoxicity assay that can measure tumor cytolysis by CTLs and NK cells in both normal culturing condition and 3D spheroid models, which would be a valuable tool in screening and assessing the efficacy of new therapeutic strategies. Data provided here demonstrate that antigen-dependent ADCC could be detected with the described methods and systems even under low antigen expression level—suggesting that the inducible reporter systems provided here can monitor ADCC activity in a sensitive manner. Further, we have provided evidence that ADCC can be quantified and monitored in both 3D tumor models and in high concentrations of patient serums with the assays described herein, which are difficult to detect due to the low sensitivity of current ADCC assays. The ability of the assays and systems here in detecting ADCC in high concentrations of patient serums suggests that they could serve as a useful tool to evaluate the potency of potential vaccines.

1. A method of evaluating the effectiveness of a cell-killing agent on a population of tumor cells, the method comprising:
a) contacting the population of tumor cells with the cell-killing agent, wherein each of the population of tumor cells comprises a nucleic acid encoding a reporter protein, wherein the expression of the nucleic acid is controlled by an inducible promoter;
b) inducing expression of the nucleic acid to produce the reporter protein; and
c) determining the amount of the reporter protein,
wherein the amount of the reporter protein negatively correlates with the effectiveness of the cell killing agent.

2. The method of claim 1, wherein the contacting step occurs before the inducing step.

3-7. (canceled)

8. The method of claim 1, wherein the inducing step comprises treating the population of tumor cells with an induction agent.

9. (canceled)

10. The method of claim 1, wherein the reporter protein is secreted by the population of tumor cells.

11-13. (canceled)

14. The method of claim 1, wherein the population of tumor cells is present in a mixture comprising a second population of cells.

15. (canceled)

16. The method of claim 1, wherein the population of tumor cells is present in a 3D spheroid or a 2D monolayer.

17. The method of claim 1, wherein the cell-killing agent is selected from the group consisting of a cytotoxin, a drug, a small molecule, a small molecule compound, and any combination thereof.

18. The method of claim 1, wherein the cell-killing agent is an immune cell.

19. The method of claim 1, wherein the cell-killing agent is an immunomodulating agent, and wherein the contacting step is conducted in the presence of an immune cell.

20. The method of claim 18, wherein the immune cell is selected from the group consisting of a natural killer (NK) cell, a natural killer T (NKT) cell, a T cell, a CAR-T cell, a CD14+ cell, a dendritic cell, a PBMC cell, and any combination thereof.

21. The method of claim 19, wherein the immunomodulating agent is an immune checkpoint inhibitor.

22. (canceled)

23. The method of claim 1, wherein the cell-killing agent is an antibody.

24. The method of claim 23, wherein the antibody is selected from the group consisting of an anti-PD-1 antibody, an anti-PD-L1 antibody, an anti-CD47 antibody, an anti-HER2 antibody, an anti-CD20 antibody, an anti-CD3 antibody, and any combination thereof.

25. The method of claim 23, wherein the antibody is multispecific.

26. The method of claim 25, wherein the antibody is an anti-HER2/anti-CD3 antibody, an anti-HER2/anti-CD47/anti-CD3 antibody, or an anti-PD-L1/anti-CD47/anti-CD3 antibody.

27. The method of claim 1, further comprising contacting the population of tumor cells with a second cell-killing agent.

28. The method of claim 27, wherein the second cell-killing agent inhibits an inhibitory checkpoint molecule selected from the group consisting of PD-1, PD-L1, PD-L2, Siglec, BTLA, CTLA-4, and any combination thereof.

29. (canceled)

30. The method of claim 28, wherein the second cell-killing agent is an siRNA or a CRISPR/Cas construct targeting the inhibitory checkpoint molecule.

31-32. (canceled)

33. The method of claim 1, wherein each of the population of tumor cells further comprises a second nucleic acid encoding a second reporter protein.

34-37. (canceled)

38. A composition comprising a population of tumor cells, wherein each of the population of tumor cells comprises a nucleic acid encoding a reporter protein, wherein the expression of the nucleic acid is controlled by an inducible promoter.

39-57. (canceled)
